(19) United States
(12) Patent Application Publication
(10) Pub. No.: US 2026/0083742 A1
(43) Pub. Date: Mar. 26, 2026

(54) HOMOLOGOUS RECOMBINATION REPAIR DEFICIENCY (HRD) AS A PREDICTIVE BIOMARKER FOR TREATING CANCER WITH WEE1 INHIBITORS

(71) Applicant: Zeno Management, Inc., San Diego, CA (US)

(72) Inventors: Jianhui Ma, San Diego, CA (US); Olivier Harismendy, San Diego, CA (US); Jiali Li, San Diego, CA (US); Ahmed Abdi Samatar, West Windsor, NJ (US); Samuel DePrimo, San Diego, CA (US); Catherine Lee, San Diego, CA (US); Monah Abed, Palo Alto, CA (US)

(21) Appl. No.: 19/404,321

(22) Filed: Dec. 1, 2025

Related U.S. Application Data
(63) Continuation of application No. PCT/US2024/031859, filed on May 31, 2024.
(60) Provisional application No. 63/506,029, filed on Jun. 2, 2023.
(60) Provisional application No. 63/599,420, filed on Nov. 15, 2023.
(60) Provisional application No. 63/635,159, filed on Apr. 17, 2024.

(51) Int. Cl. A61K 31/519; A61K 31/454; A61K 31/496; A61K 31/502; A61P 35/00

(52) U.S. Cl. CPC A61K 31/519; A61K 31/454; A61K 31/496; A61K 31/502; A61P 35/00

(57) ABSTRACT

The present disclosure provides, among other things, methods for treating cancer comprising administering an effective dose of azenosertib, or a pharmaceutically acceptable salt thereof, to subjects selected to have a homologous recombination repair deficiency (HRD).

### INCORPORATION BY REFERENCE TO PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application Nos. 63/506,029, filed Jun. 2, 2023, 63/599,420, filed Nov. 15, 2023 and 63/635,159, filed Apr. 17, 2024, each of which is hereby expressly incorporated herein by reference in their entireties. The present application is a continuation of PCT Application No. PCT/US2024/031859, filed May 31, 2024, which claims priority to U.S. Provisional Application Nos. 63/506,029, filed Jun. 2, 2023, 63/599,420, filed Nov. 15, 2023 and 63/635,159, filed Apr. 17, 2024, each of which is hereby incorporated by reference in their entireties.

### BACKGROUND

DNA damage is typically resolved by a variety of pathways and proteins that repair damaged DNA. Prominent among these pathways are proteins involved in homologous recombination, a type of DNA repair in which nucleotide sequences are exchanged between two similar or identical molecules of DNA and that usually results in error free repair of damaged DNA. However, incorrect replacement of nucleotides into DNA can cause mutations and other genetic alterations that may lead to cancer development and progression. Improper DNA repair can lead to cell death, tumor progression and cancer. Cell cycle checkpoints are important for proper DNA repair, ensuring that cells do not progress with cellular replication until their genomic integrity is restored. There remains a need for therapies capable of effectively and reliably treating cancers with DNA repair deficiencies.

### SUMMARY

The present disclosure is based in part on the discovery that homologous recombination deficiency or homologous repair deficiency (HRD) status can be used as a predictive biomarker for effective treatment of cancer using WEE1 inhibitors (e.g., azenosertib, ZN-c3). In particular, HRD status can be used as a predictive biomarker for effectively treating cancers that are resistant to other lines of therapy (e.g., chemotherapy, PARP inhibitor therapy).

The present disclosure, among other things, provides methods of treating chemotherapy-resistant cancer (e.g., platinum-resistant cancer, PARP inhibitor-resistant cancer) by administering a WEE1 inhibitor to HRD-positive subjects. In some embodiments, a WEE1 inhibitor (e.g., azenosertib) are administered in combination with one or more PARP inhibitors (e.g., olaparib, niraparib, saruparib).

For example, homologous recombination deficiency (HRD) is a biomarker present in the most advanced stages of cancer (e.g., ovarian cancer). The present disclosure provides, among other things, methods of selecting subjects or methods of selecting cancer stages or subtypes (by determining or having determined HRD status), and treating the cancer by administering a WEE1 inhibitor. The present disclosure, among other things, shows that HRD positive subjects are more responsive to azenosertib therapy, PARP inhibitor treatment or combinations thereof. Further, the present disclosure, shows that subjects that are HRD positive, but resistant to treatment with PARP inhibitors (e.g. olaparib, niraparib, saruparib), are responsive to azenosertib therapy.

DNA damage and various gene mutations lead to the development of cancer. If there is a small amount of DNA damage, the body can repair DNA leading to cell survival; however, if there is too much DNA damage, the cell triggers its own death. The body repairs damaged DNA by using tumor suppressor genes (for example, the BRCA gene, among others) and a protein called Poly (ADP-ribose) polymerase (PARP). In HRD-positive subjects (e.g., where the tumor suppressor gene is pathogenic (e.g., BRCA pathogenic mutation), cancer cells are repaired by PARP. PARP inhibitors can prevent DNA repair by PARP, which can trigger cell death, and thereby inhibits tumor growth.

WEE1 is a tumor suppressor that triggers G2/M arrest through inhibitory phosphorylation on Tyr15 of CDK1 (Cdc2) and prevents entry into mitosis to allow DNA repair during DNA damage. WEE1 inhibitors (e.g., azenosertib) also prevent DNA repair, thereby inhibiting tumor growth. Among other things, the present disclosure provides WEE1 inhibitors to treat cancers that are PARP-resistant.

Without wishing to be bound by any particular theory, it is contemplated that combined PARP and WEE1 inhibition are synergistic in controlling tumor growth (for example, in ovarian cancer, triple negative breast cancer, among others) by, for example, increasing replication stress in cancer cells, among others.

In some aspects, provided herein is a method of treating a cancer, comprising: determining or having determined whether a subject has a homologous repair deficiency (HRD) or a HRD-positive (HRD+) status, and if the subject has a HRD or a HRD-positive (HRD+) status, administering an effective dose of azenosertib, or a pharmaceutically acceptable salt thereof, wherein the administration of azenosertib, or a pharmaceutically acceptable salt thereof, results in the inhibition of the cancer in the subject.

In some embodiments, the effective dose of azenosertib, or a pharmaceutically acceptable salt thereof, is about 200 to about 450 mg/day, or about 200 to about 400 mg/day, or between about 200 to about 350 mg/day, or between about 250 to about 400 mg/day or between about 250 to about 350 mg/day, or an equivalent of any of the foregoing.

In some embodiments, the effective dose of azenosertib, or a pharmaceutically acceptable salt thereof, is at least about 250 mg/day, or an equivalent thereof.

In some embodiments, the effective dose of azenosertib, or a pharmaceutically acceptable salt thereof, is at least about 250 to about 300 mg/day, or an equivalent thereof.

In some embodiments, the effective dose of azenosertib, or a pharmaceutically acceptable salt thereof, is at least about 300 to about 350 mg/day, or an equivalent thereof.

In some embodiments, the effective dose of azenosertib, or a pharmaceutically acceptable salt thereof, is at least about 350 to about 400 mg/day, or an equivalent thereof.

In some embodiments, the effective dose is administered once a day (QD).

In some embodiments, the effective dose is administered twice a day (BID).

In some embodiments, the effective dose is administered on a continuous dosing schedule.

In some embodiments, the effective dose is administered on an intermittent dosing schedule.

In some embodiments, the intermittent dosing schedule comprises 5 days with dosing and two days without dosing in each of one or more dosing weeks. In some embodiments, the intermittent dosing schedule comprises 5 consecutive days with dosing and two consecutive days without dosing in each of one or more dosing weeks.

In some embodiments, the HRD or HRD-positive (HRD+ status) is due to the cancer having a homologous recombination repair mutation (HRRm). In some embodiments, the HRRm is a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, the HRD or HRD-positive (HRD+ status) is due to the cancer having a mutation in BRCA1 and/or BRCA2. In some embodiments, the HRD or HRD-positive (HRD+ status) is due to the cancer having a BRCA1 pathogenic mutation. In some embodiments, the BRCA1 pathogenic mutation is BRCA1^{Glu1607Ter}.

In some embodiments, the HRD or HRD-positive (HRD+ status) is due to the cancer having a homologous recombination repair reversion mutation. In some embodiments, the homologous recombination repair reversion mutation is a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, the HRD or HRD-positive (HRD+ status) is due to the cancer having a homologous recombination repair reversion mutation in BRCA1 and/or BRCA2. In some embodiments, the HRD or HRD-positive (HRD+ status) is due to the cancer having a BRCA1 splicing isoform. In some embodiments, the BRCA1 splicing isoform is a BRCA1-Δ11q splicing isoform.

In some embodiments, the cancer has an additional mutation in a gene selected from the group consisting of TP53, AKT1, BRCA2, CDKN2A, KDM6A, PTEN, RB1 and FAM35A.

In some embodiments, the method comprises administering an effective dose of azenosertib, or a pharmaceutically acceptable salt thereof, to a subject selected to have a cancer with a BRCA1/2-mutant or BRCA1/2-positive status.

In some embodiments, the method comprises administering to the subject an effective dose of azenosertib, or a pharmaceutically acceptable salt thereof, in combination with an effective dose of a PARP inhibitor, or a pharmaceutically acceptable salt thereof. In one embodiment, the PARP inhibitor is niraparib, or a pharmaceutically acceptable salt thereof. In some embodiments, an effective dose of niraparib, or a pharmaceutically acceptable salt thereof, is about 150 to about 350 mg/day, or about 150 to about 300 mg/day, or about 150 to about 250 mg/day, or about 200 to about 350 mg/day, or about 200 to about 300 mg/day, or about 200 mg/day, or about 300 mg/day or about 250 mg/day In one embodiment, the PARP inhibitor is olaparib, or a pharmaceutically acceptable salt thereof. In some embodiments, an effective dose of olaparib, or a pharmaceutically acceptable salt thereof, is about 300 to about 800 mg/day, or about 300 to about 400 mg/day, or about 600 to about 800 mg/day, or about 300 mg/day, or about 400 mg/day, or at least about 300 mg/day, or at least about 400 mg/day, or about 600 mg/day, or at least about 600 mg/day or about 800 mg/day. In one embodiment, the effective dose of olaparib, or a pharmaceutically acceptable salt thereof, is administered twice a day (BID). In some embodiments, the PARP inhibitor is a PARP1 selective inhibitor, or a pharmaceutically acceptable salt thereof. In one embodiment, the PARP1 selective inhibitor is saruparib, or a pharmaceutically acceptable salt thereof. In some embodiments, an effective dose of saruparib, or a pharmaceutically acceptable salt thereof, is about 5 to about 100 mg/day, or at least about 5 mg/day, or about 50 to about 100 mg/day, or about 50 to about 80 mg/day, or about 50 to about 70 mg/day, or about 50 to about 60 mg/day, or about 60 to about 70 mg/day or about 60 mg/day.

In some embodiments, the subject has received one or more prior lines of therapy, for example, one or more prior lines of chemotherapy (monotherapy or combination therapy) or at least one prior line with a PARP inhibitor (i.e., the subject has previously received a PARP inhibitor, or a pharmaceutically acceptable salt thereof). In one embodiment, the cancer is chemotherapy-resistant. In one embodiment, the cancer is platinum-resistant. In one embodiment, the cancer is PARP inhibitor-resistant. In one embodiment, the cancer is both chemotherapy-resistant and PARP inhibitor resistant. In one embodiment, the cancer is both platinum-resistant and PARP inhibitor resistant.

In some embodiments, the cancer is selected from the group consisting of glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colon cancer, colorectal cancer, endometrial cancer, endometrium cancer, esophagus cancer, eye cancer, gallbladder cancer, gastric cancer, gastrointestinal cancer, Hodgkin lymphoma, Non-Hodgkin lymphoma, hematological tumor, head cancer, heme malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, uterine sarcoma, uterine serous carcinoma (USC), uterine CS, vaginal cancer, vulvar cancer, Waldenstrom macroglobulinemia, Wilms tumor, solid tumor, or liquid tumor, HGSOC, invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymoma, BRAF mutant metastatic colorectal cancer and uveal melanoma.

In some embodiments, the cancer is a solid tumor or a heme malignancy.

In some embodiments, the cancer is a solid tumor.

In some embodiments, the solid tumor is ovarian cancer.

In some embodiments, the ovarian cancer is epithelial ovarian cancer.

In some embodiments, the epithelial ovarian cancer is high grade serous ovarian cancer (HGSOC).

In some embodiments, the cancer is endometrial cancer. In some embodiments, the cancer is endometrial carcinoma or endometrial cancer. In some embodiments, the cancer is uterine serous carcinoma (USC). In some embodiments, the cancer is peritoneal cancer (e.g., primary peritoneal cancer). In some embodiments, the cancer is fallopian tube cancer.

In some embodiments, the cancer is breast cancer. In one embodiment, the cancer is triple negative breast cancer. In one embodiment, the breast cancer is a HER2-expressing or a HER2-positive (HER2+) breast cancer.

In some embodiments, the cancer is prostate cancer.

In some embodiments, provided herein is a method of treating PARP inhibitor-resistant breast cancer, comprising: determining or having determined whether a subject has a homologous recombination repair deficiency (HRD) or a HRD-positive (HRD+) status, and if the subject has the HRD or the HRD-positive (HRD+) status, administering azenosertib, or a pharmaceutically acceptable salt thereof, at an effective dose, wherein the administration of azenosertib, or a pharmaceutically acceptable salt thereof, results in the inhibition of the cancer in the subject.

In some embodiments, the HRD or HRD-positive (HRD+ status) is due to the PARP inhibitor-resistant breast cancer having a BRCA1 reversion mutation.

In some embodiments, the HRD or HRD-positive (HRD+ status) is due to the PARP inhibitor-resistant breast cancer having a BRCA1 mutation. In some embodiments, the HRD or HRD-positive (HRD+ status) is due to the PARP inhibitor-resistant breast cancer having a BRCA1 splicing isoform. In some embodiments, the BRCA1 splicing isoform is a BRCA1-Δ11q splicing isoform.

In some embodiments, the cancer has an additional mutation in the gene FAM35A. In some embodiments, the cancer has an additional mutation in the gene TP53.

In some embodiments, the effective dose of azenosertib, or a pharmaceutically acceptable salt thereof, is at least about 250 mg/day, or an equivalent thereof.

In some embodiments, the effective dose of azenosertib, or a pharmaceutically acceptable salt thereof, is at least about 250 to about 300 mg/day, or an equivalent thereof.

In some embodiments, the effective dose of azenosertib, or a pharmaceutically acceptable salt thereof, is at least about 300 to about 350 mg/day, or an equivalent thereof.

In some embodiments, the effective dose of azenosertib, or a pharmaceutically acceptable salt thereof, is at least about 350 to about 400 mg/day, or an equivalent thereof.

In some embodiments, the method comprises administering to the subject an effective dose of azenosertib, or a pharmaceutically acceptable salt thereof, in combination with an effective dose of a PARP inhibitor, or a pharmaceutically acceptable salt thereof. In some embodiments, the PARP inhibitor is niraparib, olaparib, saruparib, or a pharmaceutically acceptable salt of any of the foregoing. In one embodiment, the PARP inhibitor is niraparib, or a pharmaceutically acceptable salt thereof. In some embodiments, an effective dose of niraparib, or a pharmaceutically acceptable salt thereof, is about 150 to about 350 mg/day, or about 150 to about 300 mg/day, or about 150 to about 250 mg/day, or about 200 to about 350 mg/day, or about 200 to about 300 mg/day, or about 200 mg/day, or about 300 mg/day or about 250 mg/day. In one embodiment, the PARP inhibitor is olaparib, or a pharmaceutically acceptable salt thereof. In some embodiments, an effective dose of olaparib, or a pharmaceutically acceptable salt thereof, is about 300 to about 800 mg/day, or about 300 to about 400 mg/day, or about 600 to about 800 mg/day, or about 300 mg/day, or about 400 mg/day, or at least about 300 mg/day, or at least about 400 mg/day, or about 600 mg/day, or at least about 600 mg/day or about 800 mg/day. In one embodiment, the effective dose of olaparib, or a pharmaceutically acceptable salt thereof, is administered twice a day (BID). In some embodiments, the PARP inhibitor is a PARP1 selective inhibitor, or a pharmaceutically acceptable salt thereof. In one embodiment, the PARP1 selective inhibitor is saruparib, or a pharmaceutically acceptable salt thereof. In some embodiments, an effective dose of saruparib, or a pharmaceutically acceptable salt thereof, is about 5 to about 100 mg/day, or at least about 5 mg/day, or about 50 to about 100 mg/day, or about 50 to about 80 mg/day, or about 50 to about 70 mg/day, or about 50 to about 60 mg/day, or about 60 to about 70 mg/day or about 60 mg/day.

In some embodiments, the PARP inhibitor-resistant breast cancer is niraparib-resistant.

In some embodiments, the PARP inhibitor-resistant breast cancer is olaparib-resistant.

In some embodiments, the effective dose of azenosertib, or a pharmaceutically acceptable salt thereof, is administered on an intermittent dosing schedule.

In some embodiments, the intermittent dosing schedule comprises 5 days with dosing and two days without dosing in each of one or more dosing weeks. In some embodiments, the intermittent dosing schedule comprises 5 consecutive days with dosing and two consecutive days without dosing in each of one or more dosing weeks.

In some aspects, provided herein is a method of treating chemotherapy-resistant ovarian cancer, comprising: determining whether a subject has a homologous recombination repair deficiency (HRD) or a HRD-positive (HRD+) status, and if the subject has the HRD or the HRD-positive (HRD+) status, administering an effective dose azenosertib, or a pharmaceutically acceptable salt thereof, at an effective dose, wherein the administration of azenosertib, or a pharmaceutically acceptable salt thereof, results in the inhibition of the cancer in the subject.

In some embodiments, the effective dose of azenosertib, or a pharmaceutically acceptable salt thereof, is at least about 250 mg/day, or an equivalent thereof.

In some embodiments, the effective dose of azenosertib, or a pharmaceutically acceptable salt thereof, is at least about 250 to about 300 mg/day, or an equivalent thereof.

In some embodiments, the effective dose of azenosertib, or a pharmaceutically acceptable salt thereof, is at least about 300 to about 350 mg/day, or an equivalent thereof.

In some embodiments, the effective dose of azenosertib, or a pharmaceutically acceptable salt thereof, is at least about 350 to about 400 mg/day, or an equivalent thereof.

In some embodiments, the effective dose of azenosertib, or a pharmaceutically acceptable salt thereof, is administered on an intermittent dosing schedule.

In some embodiments, the intermittent dosing schedule comprises 5 days with dosing and two days without dosing in each of one or more dosing weeks. In some embodiments, the intermittent dosing schedule comprises 5 consecutive days with dosing and two consecutive days without dosing in each of one or more dosing weeks.

In some embodiments, the chemotherapy-resistant ovarian cancer is platinum resistant.

In some embodiments, the chemotherapy-resistant ovarian cancer is further resistant to a PARP inhibitor, or a pharmaceutically acceptable salt thereof.

In some embodiments, the HRD or HRD-positive (HRD+ status) is due to the cancer having a mutation in BRCA1 and/or BRCA2.

In some embodiments, the HRD comprises a copy number variation, a somatic copy number alteration (SCNA), an aneuploidy, a loss of heterozygosity (LOH), a large-scale transition (LST) and/or a telomeric allelic imbalance (TAI).

In some embodiments, the HRD status is determined using a functional assay or genome sequencing.

In some embodiments, the HRD status is determined using an HRD score.

In some embodiments, the method further comprises determining or having determined Cyclin E expression levels in the subject.

In some embodiments, the effective dose of azenosertib, or a pharmaceutically acceptable salt thereof, is administered orally, intravenously, subcutaneously, intrathecally, intramuscularly, intracavitary, intrapleurally, intralesionally or intra-arterially.

In some embodiments, the effective dose of azenosertib, or a pharmaceutically acceptable salt thereof, is administered orally.

In some embodiments, the treatment results in a response rate at or greater than 50%.

In some embodiments, the response rate is measured by complete response (CR), partial response (PR), CA-125 50% response or combination thereof.

In some embodiments, the treatment results in progression-free survival (PFS) of 6 months or longer.

In some embodiments, the inhibition of cancer is measured by inhibition of tumor growth.

In some embodiments, the inhibition of tumor growth is measured by reduction of tumor volume.

In one aspect, provided herein is a method of treating cancer comprising, administering an effective dose of azenosertib, or a pharmaceutically acceptable salt thereof, to a subject selected to have a cancer with a homologous recombination repair deficiency (HRD).

In another aspect, provided herein is a method of treating cancer comprising, administering an effective dose of azenosertib, or a pharmaceutically acceptable salt thereof, to a subject selected to have a cancer with a homologous recombination repair deficiency-positive (HRD-positive or HRD+) status.

In some embodiments, the cancer has a homologous recombination repair mutation (HRRm). In some embodiments, the cancer has a homologous recombination repair reversion mutation.

In some embodiments, HRRm is a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, HRRm is a pathogenic mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, HRRm is a mutation likely to be pathogenic in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, HRRm is a homozygous deletion in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L.

In some embodiments, the HRD or HRD-positive (HRD+) status is due to a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, the HRD or HRD-positive (HRD+) status is due to a reversion mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, the mutation or alteration comprises any change in the gene or expression of the gene. In some embodiments, the alteration comprises dysregulation of expression. In some embodiments, dysregulation comprises promoter methylation.

In some embodiments, the HRD or HRD-positive (HRD+) status is due to the cancer having a mutation in BRCA1 and/or BRCA2. In some embodiments, HRD or HRD-positive (HRD+) status is due to a reversion mutation in BRCA1 and/or BRCA2.

In some embodiments, the cancer has an additional mutation in a gene selected from the group consisting of TP53, AKT1, BRCA2, CDKN2A, KDM6A, PTEN, RB1 and FAM35A.

In some embodiments, the HRD or HRD-positive (HRD+) status is due to the cancer having a BRCA1 splicing isoform. In one embodiment, the HRD or HRD-positive (HRD+) status is due to the cancer having a BRCA1-Δ11q splicing isoform.

In one embodiment, the method of treating cancer disclosed herein comprises administering an effective dose of azenosertib, or a pharmaceutically acceptable salt thereof, to a subject selected to have a cancer with a BRCA1/2-mutant or BRCA1/2-positive status.

In some embodiments, the HRD comprises a copy number variation, a somatic copy number alteration (SCNA), an aneuploidy, a loss of heterozygosity (LOH), a large-scale transition (LST) and/or a telomeric allelic imbalance (TAI). In some embodiments, the HRD comprises a loss of heterozygosity (LOH), a large-scale transition (LST) and/or a telomeric allelic imbalance (TAI).

In some embodiments, the HRD-positive (HRD+) status is determined using a functional assay or genome sequencing.

In some embodiments, the HRD-positive (HRD+) status is determined using an HRD score.

In some embodiments, the HRD-positive (HRD+) status is determined using a GIS score (genome instability score).

In some embodiments, the method further comprises selecting the subject based on Cyclin E1 levels. In some embodiments, the method further comprises selecting the subject based on Cyclin E1 expression. In some embodiments, the method further comprises selecting the subject based on CCNE1 gene amplification.

In some embodiments, the method further comprises selecting the subject based on levels of replication stress. In some embodiments, CCNE1 amplification or high expression increases replication stress.

In some embodiments, the method further comprises administering to the subject an effective dose of a PARP inhibitor, or a pharmaceutically acceptable salt thereof. In one embodiment, the PARP inhibitor is niraparib, or a pharmaceutically acceptable salt thereof. In one embodiment, the PARP inhibitor is olaparib, or a pharmaceutically acceptable salt thereof. In one embodiment, the PARP inhibitor is a PARP1 selective inhibitor, or a pharmaceutically acceptable salt thereof. In one embodiment, the PARP1 selective inhibitor is saruparib (AZD5305), or a pharmaceutically acceptable salt thereof. In one embodiment, the azenosertib, or a pharmaceutically acceptable salt thereof, and the PARP inhibitor, or a pharmaceutically acceptable salt thereof, are administered to the subject concurrently or concomitantly, wherein azenosertib, or a pharmaceutically acceptable salt thereof, is optionally administered on an intermittent dosing schedule. In one embodiment, the azenosertib, or a pharmaceutically acceptable salt thereof, and the PARP inhibitor, or a pharmaceutically acceptable salt thereof, are administered to the subject sequentially, wherein azenosertib, or a pharmaceutically acceptable salt thereof, is optionally administered on an intermittent dosing schedule. In some embodiments, the azenosertib, or a pharmaceutically acceptable salt thereof, and the PARP inhibitor, or a pharmaceutically acceptable salt thereof, are administered to the subject consecutively, wherein azenosertib, or a pharmaceutically acceptable salt thereof, is optionally administered on an intermittent dosing schedule. In some embodiments, the azenosertib, or a pharmaceutically acceptable salt thereof, and the PARP inhibitor, or a pharmaceutically acceptable salt thereof, are administered to the subject consecutively, wherein the azenosertib, or a pharmaceutically acceptable salt thereof, is administered prior to the PARP inhibitor, or a pharmaceutically acceptable salt thereof. In some embodiments, the azenosertib, or a pharmaceutically acceptable salt thereof, and the PARP inhibitor, or a pharmaceutically acceptable salt thereof, are administered to the subject consecutively, wherein the PARP inhibitor, or a pharmaceutically acceptable salt thereof, is administered prior to the azenosertib, or a pharmaceutically acceptable salt thereof.

In some embodiments, the subject has received one or more prior lines of therapy.

In some embodiments, the cancer is platinum-resistant.

In some embodiments, the cancer is PARP inhibitor-resistant.

In some embodiments, the subject has previously received a PARP inhibitor, or a pharmaceutically acceptable salt thereof.

In some embodiments, the effective dose of azenosertib, or a pharmaceutically acceptable salt thereof, is about 50 to about 400 mg/day, about 100 to about 400 mg/day, about 150 to about 400 mg, about 200 to about 400 mg/day, about 200 to about 375 mg/day, about 200 to about 350 mg/day, about 200 to about 300 mg/day, about 200 to about 400 mg/day, about 250 to about 450 mg/day, about 300 to about 450 mg/day or about 400 to about 600 mg/day, or equivalents thereof.

In some embodiments, the effective dose of azenosertib, or a pharmaceutically acceptable salt thereof, is about 50 mg/day, about 100 mg/day, about 150 mg/day, about 200 mg/day, about 250 mg/day, about 300 mg/day, about 325 mg/day, about 350 mg/day, about 375 mg/day, about 400 mg/day, about 450 mg/day, about 500 mg/day, about 550 mg/day or about 600 mg/day, or equivalents thereof.

In some embodiments, the effective dose of azenosertib, or a pharmaceutically acceptable salt thereof, is at least about 300 mg/day of azenosertib, or equivalents thereof. In some embodiments, the effective dose of azenosertib, or a pharmaceutically acceptable salt thereof, is at least about 350 mg/day of azenosertib, or equivalents thereof.

In some embodiments, the effective dose is about 400 mg/day of azenosertib, or equivalents thereof. In some embodiments, the effective dose is at least about 400 mg/day of azenosertib, or equivalents thereof.

In some embodiments, the effective dose of azenosertib, or a pharmaceutically acceptable salt thereof, is administered once a day, twice a day, or three times a day.

In some embodiments, the effective dose of azenosertib, or a pharmaceutically acceptable salt thereof, is administered once a day.

In some embodiments, the effective dose of azenosertib, or a pharmaceutically acceptable salt thereof, is administered as a once a day continuous dose.

In some embodiments, azenosertib, or a pharmaceutically acceptable salt thereof, is administered at an effective dose of about 400 mg/day, or an equivalent thereof, in an intermittent dosing cycle.

In some embodiments, the intermittent dosing cycle comprises five consecutive days with dosing and two days without dosing. In some embodiments, the method comprises administering azenosertib, or a pharmaceutically acceptable salt thereof, at a dose of about 400 mg/day on a 5 days on, 2 days off (5:2) weekly administration schedule. In some embodiments, the method comprises administering azenosertib, or a pharmaceutically acceptable salt thereof, at a dose of about 400 mg/day for five consecutive days with dosing and two days without dosing per week.

In some embodiments, the effective dose of azenosertib, or a pharmaceutically acceptable salt thereof, is administered orally, intravenously, subcutaneously, intrathecally, intramuscularly, intracavitary, intrapleurally, intralesionally or intra-arterially.

In some embodiments, the effective dose of azenosertib, or a pharmaceutically acceptable salt thereof, is administered orally.

In some embodiments, the treatment results in response rate at or greater than 10%, 15%, 20%, 25%, 30%, 35%, 40%, 45% or 50%.

In some embodiments, the response rate is measured by complete response (CR), partial response (PR), CA-125 50% response, or combination thereof.

In some embodiments, the treatment results in progression-free survival (PFS) of 5 months, 6 months, 7 months, 8 months, 9 months, 10 months, 11 months, 12 months or longer.

In some embodiments, the method further comprises first determining the HRD status prior to selecting the subject. In some embodiments, the method further comprises first determining the HRD status prior to treating the subject with azenosertib, or a pharmaceutically acceptable salt thereof. In some embodiments, the method further comprises determining HRD status during treatment with azenosertib, or a pharmaceutically acceptable salt thereof.

Other features, objects, and advantages are apparent in the detailed description that follows. It should be understood, however, that the detailed description, while indicating embodiments, is given by way of illustration only, not limitation. Various changes and modifications within the scope of the present disclosure will become apparent to those skilled in the art from the detailed description.

### BRIEF DESCRIPTION OF THE DRAWINGS

Drawings are for illustration purposes only and not for limitation.

FIG. 1A is a chart showing the genotype and dosage of azenosertib in subjects with cancer (e.g., uterine serous carcinoma (USC) and ovarian cancer) selected for evaluation of association of HRRm in subjects treated with 300+mg of azenosertib. LT300=lower than 300 mg of azenosertib, SD=stable disease, PR=partial response, cPR=confirmed partial response, CR=complete remission, PD=progressive disease, uPR=unconfirmed partial response (uPR), O=ovarian cancer, U=uterine cancer. Response criteria are based on Response Evaluation Criteria in Solid Tumors (RECIST) guidelines. FIG. 1B is a chart showing HRRm status in each subject and the tumor size change, in subjects receiving greater than 300 mg of azenosertib. FIG. 1C is a chart showing Best overall tumor response (BOR) and tumor size change for each subject in FIG. 1B. FIG. 1D shows stratification of subject population and survival probability of HRRm in subjects receiving at least 300 mg (300+mg) of azenosertib.

FIG. 2A-2D are graphs showing tumor volumes and tumor growth inhibition (% TGI) in mice inoculated with 22RV1 cells treated with azenosertib alone or in combination with a PARP inhibitor. FIG. 2A and FIG. 2B are graphs showing tumor volumes of 22RV1 xenografts in mice treated with azenosertib alone or in combination with niraparib. FIG. 2C and FIG. 2D are graphs showing tumor volumes of 22RV1 xenografts in mice treated with azenosertib alone or in combination with olaparib.

FIG. 3A is a graph showing tumor volumes of the HBCx-9 patient-derived xenograft (PDX) model described in Example 3. FIG. 3B and FIG. 3C are exemplary graphs showing reductions in tumor volumes of the HBCx-10 PDX model (FIG. 3B) and the HBCx-17 PDX model (FIG. 3C) following treatment with azenosertib and/or niraparib.

FIG. 4A is an exemplary graph showing tumor volumes of a BRCA1 WT PDX model (OVA2-BUR) described in Example 4. FIG. 4B is an exemplary graph demonstrating change in tumor volume of a BRCA1 mutation pathogenic CTG-0703 PDX model after treatment with azenosertib and/or niraparib. FIG. 4C is an exemplary graph demonstrating tumor volumes of a BRCA1 benign mutant (CTG-2213) PDX model after treatment with azenosertib and/or niraparib.

FIG. 5A-5C are exemplary graphs showing % inhibition of WEE1 inhibitors (azenosertib and AZD1775) or PARP inhibitors (niraparib and olaparib) in PARP inhibitor-sensitive and resistant triple negative breast cancer cell lines in vitro. FIG. 5D-5G are exemplary graphs showing tumor volumes in animals after treatment with WEE1 inhibitors alone or in combination with a PARP inhibitor in a PARP inhibitor-sensitive model and two PARP inhibitor-resistant models. Parental MDA-MB-436 (TP53, BRCA1 mutant) remains sensitive to PARP inhibitors (FIG. 5A and FIG. 5D), whereas MDA-MB-436 Nir^{R} (TP53, BRCA1m Reversion) (FIG. 5B and FIG. 5E) and MDA-MB-436 Ola^{R} (TP53, BRCA1m Reversion) (FIG. 5C, FIG. 5F and FIG. 5G) are resistant to PARP inhibitors.

FIG. 6 is an exemplary pathological scan of a subject with HRD+ PARP inhibitor-Platinum-Resistant Ovarian Cancer at screening and after treatment with azenosertib.

FIGS. 7A-7B are graphs analyzing overall survival of subjects with USC who are HRRm or HRRwt, and selected to have a TP53-mutated status, with the overall survival measured since sample collection or since initiation of the carboplatin treatment.

FIGS. 8A-8E are graphs showing in vitro cell viability and tables showing IC₅₀ values after treatment with azenosertib, olaparib, or AZD5305 in the cell lines UWB1289, HCC1937, COV362, HCC1569 and HCC1395

FIG. 9 is a graph showing tumor volumes and tumor growth inhibition (% TGI) in mice inoculated with PARP inhibitor-resistant HCC1937 triple negative breast cancer (TNBC) cells after treatment with azenosertib monotherapy or in combination with olaparib, either dosed concurrently or on alternating weeks.

FIGS. 10A-10B are graphs showing tumor volumes and tumor growth inhibition (% TGI) in mice inoculated with PARP inhibitor-resistant SUM149PT triple negative breast cancer (TNBC) cells after treatment with azenosertib monotherapy or in combination with olaparib or AZD5305, dosed concurrently.

### DEFINITIONS

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, the term “about” has its usual meaning as understood by those skilled in the art and thus indicates that a value includes the inherent variation of error (e.g., ±10%) for the method being employed to determine a value, or the variation that exists among multiple determinations.

As used herein, the terms “modify” or “alter”, or any forms thereof, mean to modify, alter, replace, delete, substitute, remove, vary, or transform.

As used herein, the terms “function” and “functional” have their usual meaning as understood by those skilled in the art and thus refer to a biological, enzymatic, or therapeutic function.

As used herein, the term “endogenous” has its usual meaning as understood by those skilled in the art and thus refers to the native, or wild type property of a gene, protein, or cell. In some embodiments, the endogenous gene is the wild type sequence of said gene. In some embodiment, the endogenous protein is the wild type sequence of said protein. In some embodiments, the endogenous protein function is the wild type function and activity level of said protein. In some embodiments, the endogenous cell is the wild type cell.

The term “mutation” has its usual meaning as understood by those skilled in the art and refers to an alteration of genetic sequence. In some embodiments, cells have multiple mutations. In some embodiments, mutations are in coding regions of the genome. Mutations can range in size from a single base-pair, to a large segment of the chromosome that includes multiple genes. In some embodiments, at least one mutation is silent. In some embodiments, the mutation may have no significant impact on gene expression or function. In some embodiments, at least one mutation has an impact on gene expression or function, such as gene amplification, overexpression, or enhanced copy number. In some embodiments, at least one mutation is silent (e.g., not changing the coding sequence). In some embodiments, at least one mutation has a small impact on protein expression or function. In some embodiments, at least one mutation has a moderate impact on protein expression or function. In some embodiments, at least one mutation has a large impact on protein expression or function. In some embodiments, at least one mutation prevents protein expression or function. Non-limiting examples of mutations include insertions, deletions, truncations, substitutions, duplications, translocations, and inversions. In some embodiments, mutations are “somatic,” or occurring in body cells and are not inheritable. In some embodiments, a subset of somatic cells in an organism have at least one mutation that other somatic cells do not have. In some embodiments, mutations are “germline,” or occurring in germ cells and are inheritable.

As disclosed herein, mutations can be monitored through a variety of sequencing, expression, or functional assays. Non-limiting examples include DNA sequencing, RNA sequencing, DNA hybridization, protein sequencing, targeted genomic sequencing, whole exome sequencing, whole genome sequencing, ATAC-sequencing, Sanger sequencing, PCR, qPCR, RT-PCR, RT-qPCR, Next Generation Sequencing, protein truncation test, DNA microarrays, heteroduplex analysis, denaturing gradient gel electrophoresis, nucleotide sequencing, single strand conformational polymorphism, restriction enzyme digestion assay, fluorescence in situ hybridization (FISH), comparative genomic hybridization, restriction fragment length polymorphism, amplification refractory mutation system PCR, nested PCR, multiplex ligation-dependent probe amplification, single strand conformational polymorphism, and oligonucleotide ligation assay. Mutations can also be monitored through a variety of antibody-based methods using biological samples including, but are not limited to, Western blotting, fluorescence activated cell sorting, immunofluorescence, immunohistochemistry, immunocytochemistry, immunoprecipitation, enzyme-linked immunosorbent assay, radioimmunoassays, and electrochemiluminescence assays.

The term “cancer” is used herein in its usual biological sense and understood by those skilled in the art. Thus, it can include the cancer of any cell type, such as but not limited to glioblastoma, astrocytoma, meningioma, craniopharyngioma, medulloblastoma, and other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, heme malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma, vaginal cancer, vulvar cancer, Waldenstrom macroglobulinemia, Wilms tumor, solid tumor, or liquid tumor.

As used herein, the term “tumor” has its usual meaning as understood by those skilled in the art and refers to an abnormal growth of cells or tissue. In some embodiments, the tumor is benign. In some embodiments, the tumor is malignant. A tumor becomes a cancer when it metastasizes, or spreads to other areas of the body. The term “solid tumor” as used herein has its usual meaning as understood by those skilled in the art and refers to an abnormal mass of tissue that does not contain liquid areas or cysts. Non-limiting examples of solid tumors include sarcomas, carcinomas, or lymphomas. Many cancer tissues can form solid tumors, such as but not limited to breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer. The terms “cancer” and “tumor” may generally be used interchangeably unless the context clearly indicates that a more specific meaning is intended.

The term “cell” as used herein has its usual meaning as understood by those skilled in the art and can refer to any cell type. In some embodiments, said cells are mammalian cells. In some embodiments, said cells are human cells.

The terms “individual”, “subject”, or “subject” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea or pigs. In some embodiments, the subject can be human. In some embodiments, the subject can be a child and/or an infant. In other embodiments, the subject can be an adult.

The term “cancer treatment” as used herein has its usual meaning as understood by those skilled in the art and refers to a therapeutic modality (such as surgery and/or radiation) or an anti-cancer agent such as a small molecule, compound, protein, or other medicant that is used to treat, inhibit, or prevent cancer. Non-limiting examples of common classes of anti-cancer agents usable with any one or more of the alternatives described herein include alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators, immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-Jun-N-terminal kinase (INK) inhibitors, mitogen-activated protein kinase (MAPK) inhibitors, MDM2 inhibitors, MEK inhibitors, ERK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinon, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin, or verteporfin. Examples of chemotherapeutic agents useful for cancer treatment include carboplatin, cisplatin, paclitaxel, docetaxel, pegylated liposomal doxorubicin, doxorubicin, gemcitabine, cytarabine, fludarabine, fluorouracil (5-FU), irinotecan, topotecan, temozolomide, triapine, 5-azacytidine, capecitabine, AraC-FdUMP[10](CF-10), cladribine, decitabine, hydroxyurea and oxaliplatin, or a pharmaceutically acceptable salt of any of the foregoing. Other examples of chemotherapeutic agents useful for cancer treatment include azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and vincristine, or a pharmaceutically acceptable salt of any of the foregoing.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the compounds described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof, and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

As used herein, the term “an equivalent thereof” or “an equivalent of any of the foregoing” refers to an effective dose as described above of the compound e.g., azenosertib in other salt forms.

The term “break” or “break days” refers to a time period when azenosertib, or a pharmaceutically acceptable salt thereof, is not administered or days without dosing, days off therapy, or break days. For example, break refers to a period subsequent to a dosing cycle or an intervening period when azenosertib dosing is paused between dosing weeks.

The term “platinum-resistant cancer” refers to a cancer that responds at first to treatment with drugs that contain the metal platinum (such as but not limited to carboplatin, cisplatin, and oxaliplatin), but then comes back within a certain period. For example, ovarian cancer that comes back within 6 months after treatment is considered platinum-resistant. The term “platinum-resistant cancer” also refers to a cancer that is resistant at the beginning of treatment with drugs that contain platinum and does not respond to drugs that contain platinum during treatment with the same.

The term “PARP inhibitor-resistant cancer” refers to a cancer that responds at first to treatment with at least one PARP inhibitor (such as but not limited to olaparib, niraparib, and AZD5305), but then comes back within a certain period. For example, ovarian cancer and/or triple negative breast cancer that comes back within 6 months after treatment is considered PARP inhibitor-resistant. The term “PARP inhibitor-resistant cancer” also refers to a cancer that is resistant at the beginning of treatment with at least one PARP inhibitor and does not respond to respond to the PARP inhibitor(s) during treatment with the same.

As used herein, “pathogenic mutation” refers to a mutation that increases risk of developing certain types of cancer. For example, “BRCA pathogenic mutation” refers to BRCA1 or BRCA2 variants which increases risks of developing cancer, e.g., germline pathogenic variants in BRCA1 BRCA2 are associated with ovarian cancer, fallopian tube cancer, primary peritoneal cancer, male breast cancer, prostate cancer, pancreatic cancer and early-onset breast cancer. BRCA1/2 pathogenic mutations are associated with 45%-85% greater risk of developing breast cancer and/or 10%-46% greater risk of developing ovarian cancer. There are several BRCA1/2 pathogenic variants known in the art disclosed at BRCA Exchange (https://www.brcaexchange.org/variants?search=pathogenic), incorporated by reference herein in entirety.

Various aspects are described in detail in the following sections. The use of sections is not meant to limit the present disclosure. Each section can apply to any aspect of present disclosure. In this application, the use of “or” means “and/or” unless stated otherwise.

### DETAILED DESCRIPTION

The present disclosure, provides among other things, HRD or HRD status as a predictive biomarker for treating methods of treating cancer, by administering a WEE1 inhibitor, or a pharmaceutically acceptable salt thereof (e.g., azenosertib, or a pharmaceutically acceptable salt thereof, that is also referred to as ZN-c3), to homologous recombination deficient (HRD)-positive subjects, and the HRD-positive (HRD+) status can be used as a predictive biomarker for treating such cancer. The present disclosure, among other things, provides methods of treating chemotherapy-resistant cancer (e.g., platinum-resistant cancer, PARP inhibitor-resistant cancer) by administering a WEE1 inhibitor, or a pharmaceutically acceptable salt thereof, to HRD-positive (HRD+) subjects. In some embodiments, a WEE1 inhibitor, or a pharmaceutically acceptable salt thereof, (e.g. azenosertib, or a pharmaceutically acceptable salt thereof) is administered in combination with a PARP inhibitor, or a pharmaceutically acceptable salt thereof (e.g. olaparib, niraparib, saruparib, or a pharmaceutically acceptable salt of any of the foregoing).

In some aspects, provided herein is a method of treating cancer, comprising: determining or having determined whether a subject has a homologous repair deficiency (HRD), and if the subject is HRD+, administer an effective dose of azenosertib, or a pharmaceutically acceptable salt thereof, wherein the administration of azenosertib, or a pharmaceutically acceptable salt thereof, results in the inhibition of the cancer in the subject.

In some aspects, provided herein is a method of treating PARP inhibitor-resistant breast cancer, comprising: determining or having determined whether a subject in need of treatment has a homologous recombination repair deficiency (HRD), and if the subject is HRD+, administering azenosertib, or a pharmaceutically acceptable salt thereof, at an effective dose, and wherein the administration of azenosertib, or a pharmaceutically acceptable salt thereof, results the in inhibition of the PARP inhibitor-resistant breast cancer in the subject. In some embodiments, the PARP inhibitor-resistant breast cancer is niraparib-resistant. In some embodiments, the PARP inhibitor-resistance breast cancer is olaparib-resistant.

In some aspects, provided herein is a method of treating chemotherapy-resistant ovarian cancer, comprising: determining whether a subject in need of treatment has a homologous recombination repair deficiency (HRD), and if the subject is HRD+, administering azenosertib, or a pharmaceutically acceptable salt thereof, at an effective dose, wherein the administration of azenosertib, or a pharmaceutically acceptable salt thereof, results in the inhibition of the chemotherapy-resistant ovarian cancer in the subject.

In some embodiments, the chemotherapy-resistant ovarian cancer is platinum resistant.

In some embodiments, the chemotherapy-resistant ovarian cancer is further resistant to a PARP inhibitor, or a pharmaceutically acceptable salt thereof.

The present disclosure provides, among other things, methods for treating cancer in a subject selected to have a homologous recombination repair deficiency (HRD).

The present disclosure further provides methods of treating cancer comprising administering an effective dose of azenosertib, or a pharmaceutically acceptable salt thereof, to a subject selected as having a homologous recombination repair deficiency (HRD).

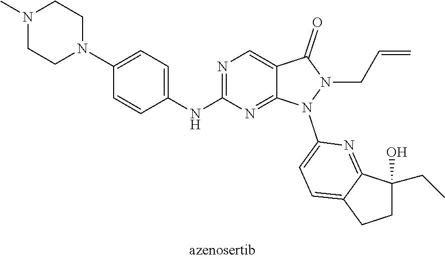 [SMILES: C=CCn1c(=O)c2cnc(Nc3ccc(N4CCN(C)CC4)cc3)nc2n1-c1ccc2c(n1)[C@@](O)(CC)CC2]

The compound azenosertib, and pharmaceutically acceptable salts thereof, are WEE1 inhibitors. The chemical structure of the compound azenosertib is depicted above. The compound azenosertib and pharmaceutically acceptable salts thereof, can be prepared in various ways. See, e.g., WO 2019/173082. WO 2019/173082 and WO 2021/231653 describe the compound azenosertib and methods of using it to treat cancer. These terms may be used interchangeably herein.

WEE1 is a tyrosine kinase that is a critical component of the ATR-mediated G2 cell cycle checkpoint control that prevents entry into mitosis in response to cellular DNA damage. WEE1 activation can lead to the selective phosphorylation of CDK2, thereby regulating CDK2-cyclin A/E complexes which control the G1/S phase progression. Inhibition of WEE1 can result in excessive replication activity, thereby leading to replication catastrophe. WEE1 inhibition has the potential to sensitize tumors to induce tumor cell death.

In one aspect, provided herein are methods of treating cancer comprising administering an effective dose of a WEE1 inhibitor to a subject selected as having a homologous recombination repair deficiency (HRD). Examples of additional WEE1 inhibitors for use in the methods described herein include those described in the following publications: WO 2019/074979, WO 2020/210383, WO 2020/210375, WO 2020/210377, WO 2020/210380, WO 2020/210381, WO 2022/082174, U.S. 2022/0162229, U.S. 2022/0168313, U.S. 2022/0169646, U.S. 2022/0220115, U.S. Pat. No. 11,332,473, WO 2019/173082, WO 2019/011228, WO 2019/138227, WO 2018/162932, WO 2018/011570, WO 2018/011569, US 2022/0194947, WO 2018/090939, WO 2019/011228, U.S. 2019/0308984, U.S. 2020/0131192, WO 2021/073491, WO 2022/188802, U.S. Pat. Nos. 11,345,710, 11,345,711, WO 2015/092431, WO 2015/019037, WO 2014/167347, WO 2007/126122, WO 2011/034743, U.S. 2007/0254892, WO 2008/133866, U.S. 2016/0060258, WO 2019/085933, WO 2020/221358, EP 3712150, WO 2018/133829, WO 2021/047627, US 2021/0403451, WO 2020/083404, US 2022/0324848, WO 2019/037678, WO 2018/171633, CN 113387962, WO 2019/165204, WO 2012/161812, WO 2013/012681, WO 2013/013031, WO 2013/059485, WO 2013/126656, U.S. 2012/0220572, U.S. 2013/0018045, KR 2016035878, KR 2020016567, WO 2018/056621, WO 2017/075629, WO 2019/169065, WO 2019/134539, WO 2020/028814, US 2021/0309630, WO 2020/069105, WO 2020/192581, U.S. 2022/0194960, CN 114831993, CN 115073466, CN 111718348, WO 96/34867, WO 2008/153207, WO 2010/067888, WO 2009/054332, WO 2021/074251, CN 112142763, WO 2020/259724, U.S. 2022/0259210, WO 2019/096322, CN 112142747, CN 112142748, US 2022/0324868, WO 2021/043152, WO2021/254389, WO 2022/171088, WO 2022/171126, WO 2022/171128, WO 2022/174765, WO 2022/174796, CN 112442049, CN 114072411, CN 113402520, CN113387962, KR 2022081171, WO 2022/124748, WO 2022/155202, CN 114591334, CN 113402250, WO 2021/074251 and CN 115197221, each of which is hereby incorporated by reference in their entireties.

In some embodiments, the WEE1 inhibitor is selected from AZD1775, SC0191, PD0166285, NUV-569, STC-8123, IMP7068, Debio 0123, SY-4835, SPH-6162, APR-1051 (formerly ATRN-W1051), SGR-3515 and ACR-2316, or any combination thereof (including pharmaceutically acceptable salts of any of the foregoing).

In other embodiments, the WEE1 inhibitor is a molecule as shown below, or a pharmaceutically acceptable salt or N-oxide thereof.

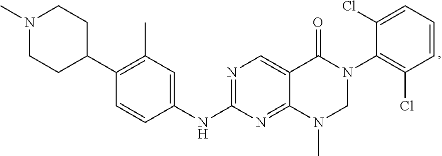 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof or an N-oxide thereof.

In some embodiments, the WEE1 inhibitor is selected from the compounds

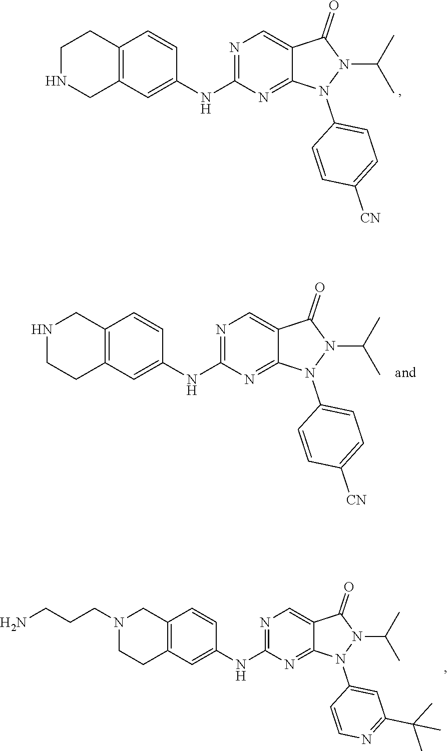 [SMILES: CC(C)n1c(=O)c2cnc(Nc3ccc4c(c3)CNCC4)nc2n1-c1ccc(C#N)cc1 ; CC(C)n1c(=O)c2cnc(Nc3ccc4c(c3)CCNC4)nc2n1-c1ccc(C#N)cc1 ; CC(C)n1c(=O)c2cnc(Nc3ccc4c(c3)CCN(CCCN)C4)nc2n1-c1ccnc(C(C)(C)C)c1]

or a pharmaceutically acceptable salt of any of the foregoing.

In some embodiments, the WEE1 inhibitor is selected from the compounds below:

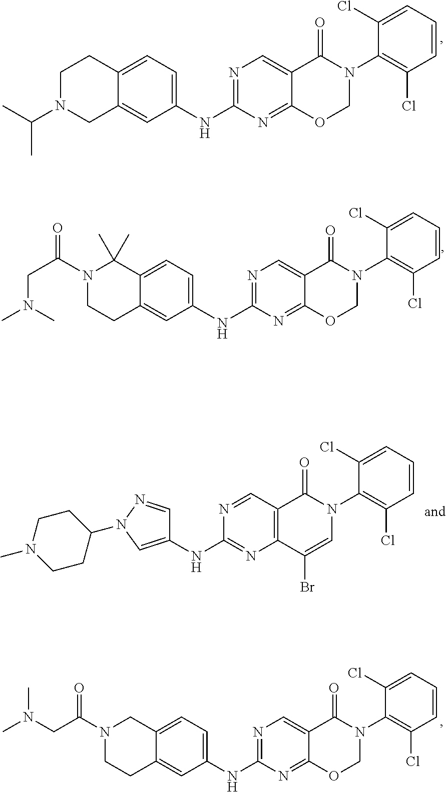 [SMILES: CC(C)N1CCc2ccc(Nc3ncc4c(n3)OCN(c3c(Cl)cccc3Cl)C4=O)cc2C1 ; CN(C)CC(=O)N1CCc2cc(Nc3ncc4c(n3)OCN(c3c(Cl)cccc3Cl)C4=O)ccc2C1(C)C ; CN1CCC(n2cc(Nc3ncc4c(=O)n(-c5c(Cl)cccc5Cl)cc(Br)c4n3)cn2)CC1 ; CN(C)CC(=O)N1CCc2cc(Nc3ncc4c(n3)OCN(c3c(Cl)cccc3Cl)C4=O)ccc2C1]

or a pharmaceutically acceptable salt of any of the foregoing.

In some embodiments, the WEE1 inhibitor is the compound below.

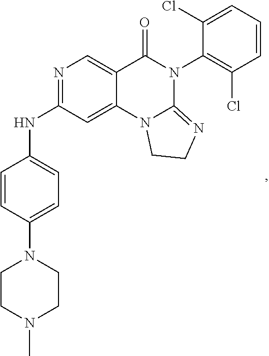 [SMILES: CN1CCN(c2ccc(Nc3cc4c(cn3)C(=O)N(c3c(Cl)cccc3Cl)C3=NCCN34)cc2)CC1]

or a pharmaceutically acceptable salt thereof.

In some embodiments, the WEE1 inhibitor is selected from the compounds below:

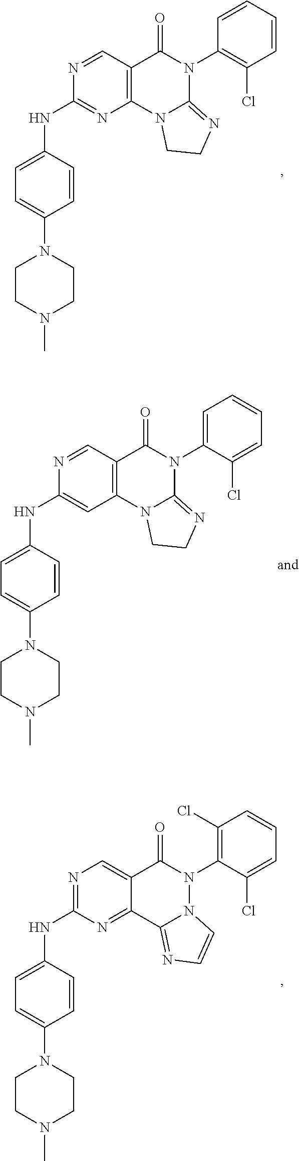 [SMILES: CN1CCN(c2ccc(Nc3ncc4c(n3)N3CCN=C3N(c3ccccc3Cl)C4=O)cc2)CC1 ; CN1CCN(c2ccc(Nc3cc4c(cn3)C(=O)N(c3ccccc3Cl)C3=NCCN34)cc2)CC1 ; CN1CCN(c2ccc(Nc3ncc4c(=O)n(-c5c(Cl)cccc5Cl)n5ccnc5c4n3)cc2)CC1]

or a pharmaceutically acceptable salt of any of the foregoing.

### Homologous Recombination Deficiency (HRD)

Homologous recombination (HR) is a conserved DNA repair process that enables the exchange of genetic information between identical or closely related DNA molecules. HR coordinates high-fidelity repair of double-stranded DNA breaks (DSBs), and functions primarily during the late S and G2 phases of the cell cycle to exploit the intact sister chromatid as a template for error-free repair. It is most widely used by cells to accurately repair harmful breaks (i.e., DNA damage) that occur on both strands of DNA (e.g., double strand breaks (DSB)). DNA damage may occur from exogenous (external) sources like UV light, radiation, or chemical damage; or from endogenous (internal) sources like errors in DNA replication or other cellular processes that create DNA damage. The most common lesions in cell DNA are single strand breaks (SSB), happening in tens of thousands per cells per day. DNA damage resulting in double strand breaks (DSB) are typically repaired by HR processes.

A deficiency in homologous recombination may result in the utilization of other pathways for DNA repair, such as non-homologous end-joining (NHEJ). However, NHEJ is more error-prone compared to homologous recombination in DNA repair, resulting in a greater number of mutations and thus increasing the risk of chromosomal instability and tumor transformation.

Homologous recombination deficiency (“HR deficiency” or “HRD” or Homologous Recombination Repair Defective) is a disease state arising in tumors through loss of the homologous recombination DNA repair pathway. HRD is a phenotype that is characterized by the inability of a cell to effectively repair DNA double-strand breaks using the HRR pathway. As described in more detail below, the terminology “HRD” and “HRP” refer to the presence or absence of an HRD phenotype. Cancers can be characterized as homologous recombination deficient (also referred to as homologous repair deficient, HR deficiency, HRD, HRD positive (HRD+). Alternatively, a cancer can be characterized as homologous recombination proficient (also referred to as homologous repair proficient, or HRP or HRD negative (HRD−)).

HRD is a term for which various synonyms exists in the art and such terms may be used interchangeably herein, including in particular HRD-positive (HRD+) (meaning a cancer is characterized as being HRD). Similarly, terms such as not-HRD and HRP, including in particular HRD-negative (HRD−) (meaning a cancer is not characterized as being HRD). The use of the terms HRD, not-HRD and HRP herein are to be read accordingly.

If cancer cells have HRD (e.g., deficient HR), the chances of the cell recovering from the DSB lowers, leading the cell into apoptosis (programmed cell death), instead of the cell continuing to proliferate.

In some embodiments, HRD may occur through changes in a gene selected from BRCA1, BRCA2, ATM, BARD1, BRIP1, CDK12, CHEK1, CHEK2, FANCL, PALB2, RAD51B, RAD51C, RAD51D and RAD54L. In some instances, HRD can occur through biallelic inactivation of BRCA1 and/or BRCA2. In some embodiments, HRD may occur through changes in RAD51C. In some embodiments, HRD may occur through changes in ATM. In some embodiments, HRD may occur through changes in BARD1. In some embodiments, HRD may occur through changes in BRIP1. In some embodiments, HRD may occur through changes in CDK12. In some embodiments, HRD may occur through changes in CHEK1. In some embodiments, HRD may occur through changes in CHEK2. In some embodiments, HRD may occur through changes in FANCL. In some embodiments, HRD may occur through changes in PALB2. In some embodiments, HRD may occur through changes in RAD51B. In some embodiments, HRD may occur through changes in RAD51C. In some embodiments, HRD may occur through changes in RAD51D. In some embodiments, HRD may occur through changes in RAD54L.

In some embodiments, changes in the gene comprises changes in gene expression. In some embodiments, changes in the gene comprises changes in gene sequence.

HRD may be detected in DNA sequencing data by counting certain characteristic megabase-scale copy number alterations that accumulate over time in the absence of HR repair. HRD may also occur as a result of a large number of genetic lesions.

### Subject History and Selection

As described herein, subjects may be selected based changes in a gene selected from BRCA1, BRCA2, ATM, BARD1, BRIP1, CDK12, CHEK1, CHEK2, FANCL, PALB2, RAD51B, RAD51C, RAD51D and RAD54L.

In some embodiments, the gene is BRCA1. In some embodiments, the gene is BRCA2. In some embodiments, the BRCA1 mutation is a BRCA1 pathogenic mutation. In some embodiments, the BRCA1 pathogenic mutation is BRCA1^{Glu1607Ter}. In some embodiments, the HRD is a BRCA1 reversion mutation. In some embodiments, the HRD is a BRCA1 splice variant. In some embodiments, the BRCA1 splice variant is a BRCA1-Δ11q mutant.

In some embodiments, the gene is RAD51C. In some embodiments, the gene is ATM. In some embodiments, the gene is BARD1. In some embodiments, the gene is BRIP1. In some embodiments, the gene is CDK12. In some embodiments, the gene is CHEK1. In some embodiments, the gene is CHEK2. In some embodiments, the gene is FANCL. In some embodiments, the gene is PALB2. In some embodiments, the gene is RAD51B. In some embodiments, the gene is RAD51C. In some embodiments, the gene is RAD51D. In some embodiments, the gene is RAD54L.

In some embodiments, the subject has a BRCA1/2 mutation (germline or somatic)-associated epithelial ovarian, fallopian tube or primary peritoneal cancer.

HRD status may be due to a point mutation or other genetic changes such as a copy number variation, a somatic copy number alteration (SCNA), an aneuploidy, a loss of heterozygosity (LOH), a large-scale transition (LST) and/or a telomeric allelic imbalance (TAI).

In some embodiments, the subject has HRD due to a copy number variation, a somatic copy number alterations (SCNA), an aneuploidy, a loss of heterozygosity (LOH), a large-scale transition (LST) and/or a telomeric allelic imbalance (TAI).

In some embodiments, the subject has HRD due to a copy number variation. In some embodiments, the subject has HRD due to a somatic copy number alterations (SCNA). In some embodiments, the subject has HRD due to an aneuploidy. In some embodiments, the subject has HRD due to a loss of heterozygosity (LOH). In some embodiments, the subject has HRD due to a large-scale transition (LST). In some embodiments, the subject has HRD due to a telomeric allelic imbalance (TAI).

In some embodiments, the method further comprises first determining the HRD or HRRm biomarker status prior to the selecting step.

In some embodiments, the subject has received one or more prior lines of therapy. In some embodiments, the subject has received 2 prior lines of therapy. In some embodiments, the subject has received 3 prior lines of therapy. In some embodiments, the subject has received 3 or more prior lines of therapy. In some embodiments, the subject has received one or more prior lines of therapy that includes at least one PARP inhibitor, whether as monotherapy or as combination therapy with a chemotherapeutic agent other than a WEE1 inhibitor (such as azenosertib).

In some embodiments, the subject has a cancer that is relapsed or refractory. In some embodiments, the subject is platinum-resistant. In some embodiments, the subject is platinum-refractory.

### Methods of Determining HRD Status

Subject selection or cancer type characterization may be based on HRD biomarker status using a variety of methods known in the art. In some embodiments, HRD is characterized by a deleterious or suspected deleterious BRCA mutation and/or genomic instability. In some embodiments, HRD is characterized by a deleterious or suspected deleterious BRCA mutation. In some embodiments, HRD is characterized by a genomic instability.

In some embodiments, HRD status is assessed by the mutation status of BRCA1/2 and/or specific patterns of genomic instability—measured by the evaluation of a combination of one or more genome-wide measures to derive a HRD related genomic instability score (e.g., gLOH+TAI+LST). In some embodiments, HRD status is defined by the mutation status of BRCA1/2 and/or genomic instability as measured by gLOH.

In some embodiments, the HRD status is an absolute value or standard. In some embodiments, the HRD status is determined based on whether the subject has a gene alteration in one or more genes in the HR pathway.

In some embodiments, HRD status is determined using information derived from RNA sequencing and DNA sequencing of cancerous tissue. In some embodiments, DNA sequencing data from matched cancerous tissue and germline tissue are used together to determine HRD status. In some embodiments, HRD status is determined based on predictions using RNA or DNA sequencing information.

In some embodiments, HRD status is determined using mRNA transcription data generated from a cancerous tissue or a subject. In some embodiments, HRD status is determined using genome-wide loss of heterozygosity (gLOH) determined from DNA sequencing data generated from a cancerous tissue. In some embodiments, HRD status is determined using DNA sequencing from a matched non-cancerous tissue.

In some embodiments, HRD status is determined using both RNA and DNA sequencing data.

In some embodiments, HRD negative status is classified as a genetic profile with no single nucleotide variants, no short insertions or deletions and/or diploid copy number of BRCA1 and BRCA2 genes.

In some embodiments, HRD positive status is classified as containing a nucleotide variant, insertion, or deletion in a gene selected from BRCA1, BRCA2, ATM, BARD1, BRIP1, CDK12, CHEK1, CHEK2, FANCL, PALB2, RAD51B, RAD51C, RAD51D and RAD54L.

In some embodiments, HRD positive status is classified as including one or more features of the RNA and/or DNA sequencing data (e.g., one or more of mRNA expression levels for a plurality of genes, a measure of loss of genomic heterozygosity, a measure of genomic and/or transcriptomic rearrangements (e.g., one or more of insertions, deletions, gene fusions, inversions, etc.), a measure of genomic methylation, etc.).

In some embodiments, testing for HRD variants is based known pathogenic variants in the germline sequence of one or more HR associated genes (e.g., BRCA1, BRCA2, ATM, BARD1, BRIP1, CDK12, CHEK1, CHEK2, FANCL, PALB2, RAD51B, RAD51C, RAD51D and/or RAD54L).

### HRRm Mutations

Methods provided herein comprise selecting subjects based on HRRm status. In some embodiments, subjects are selected for treatment with a WEE1 inhibitor (e.g., azenosertib, or a pharmaceutically acceptable salt thereof) based on mutations in one or more of the following genes used to determine HRRm status: BRCA1, BRCA2, ATM, BARD1, BRIP1, CDK12, CHEK1, CHEK2, FANCL, PALB2, RAD51B, RAD51C, RAD51D and/or RAD54L.

In some embodiments, testing for HRRm status is based on the presence of known pathogenic variants in the germline sequence of one or more HR associated genes (e.g., BRCA1, BRCA2, ATM, BARD1, BRIP1, CDK12, CHEK1, CHEK2, FANCL, PALB2, RAD51B, RAD51C, RAD51D and/or RAD54L).

In some embodiments, HRD is due to an alteration in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L. In some embodiments, alteration in a gene comprises a function, activity, or expression related change. In some embodiments, alteration in a gene comprises a pathogenic or deleterious change.

HRRm status may be determined by an assay such as e.g., FoundationOne® CDx, Myriad (MyChoice® CDx), Tempus xT HRD, Caris Molecular. Intelligence Comprehensive Tumor Profiling or any other CLIA certified (or local equivalent) lab may also be used. Confirmed deleterious mutations in at least 1 of the genes involved in HRR as determined from CLIA-approved (or country-specific equivalent) prior genomic profiling.

### HRD Diagnostic Methods

In some embodiments, HRD status is determined by a diagnostic assay. In some embodiments, HRD status is determined using a genotypic assay. In some embodiments, HRD status is determined using a phenotypic assay. HRD status can be determined using any method known in the art. For example, exemplary methods are described in Stewart M D et al., Oncologist. 2022 Mar. 11; 27(3):167-174. (PMID: 35274707; PMCID: PMC8914493), which is hereby incorporated by reference in its entirety.

In some embodiments, the diagnostic test is a molecular (sequence-) based in vitro diagnostic test for the HRD status of a cancer. In some embodiments, clinical-grade HRD assays detect ‘genomic scars’, which may be used as a measure of HRD. An HRD genomic scar assay evaluates for the percentage of genomic regions with LOH determined through tumor single-nucleotide polymorphism (SNP) sequencing (FoundationOne CDx, Foundation Medicine) or through a genomic instability score (GIS) calculated by combining three factors obtained from allele-specific copy number profiles for SNP-LOH, telomeric allelic imbalance (TAI), and large-scale transitions (LSTs) (myChoice CDx, Myriad Genetics; HRDsig, Foundation Medicine).

In some embodiments, the HRD diagnostic assay is a companion diagnostic used for treatment with a PARP inhibitor. For example, in some embodiments, the diagnostic test is a molecular (sequence-) based in vitro diagnostic test (“MyChoice”) for the HRD status of a cancer available from Myriad Genetics, Inc. This test is approved by the FDA (USA) as a companion diagnostic for use of the PARP inhibitor niraparib.

In some embodiments, HRD is defined by tumor BRCA mutation or a composite genomic instability score of greater than or equal to 42 (a cancer is characterized as HRD if the test score (HRD score) is at least 42, otherwise it is characterized as not-HRD (HRP)).

In some embodiments, HRD score is determined by BRCA1/2 mutation status. In some embodiments, BRCA1/2 mutation is denoted as homologous recombination deficiency (HRD) positivity. In some embodiments, the diagnostic method is the “MyChoice” diagnostic test of Myriad Genetics, Inc. In some embodiments, HRD status is based on a predetermined threshold. In some embodiments, the threshold is a test score (HRD score) of 42 for characterizing (or classifying) a cancer as HRD or HRP (not-HRD).

In some embodiments, the diagnostic method includes the % LOH score measuring the percentage of genomic LOH as a marker of HRD positivity, % LOH≥16 is denoted as % LOH-high. In some embodiments, the diagnostic method is the FoundationOne CDx (Foundation Medicine). In some embodiments, the FoundationOne CDx (Foundation Medicine) and includes the % LOH score measuring the percentage of genomic LOH as a marker of HRD positivity, % LOH≥16 is denoted as % LOH-high.

### HRD Score

HRD positive status may be classified using an HRD score. HRD score may be determined by considering one or more features of the RNA and/or DNA sequencing data (e.g., one or more of mRNA expression levels for a plurality of genes, a measure of loss of genomic heterozygosity, a measure of genomic and/or transcriptomic rearrangements (e.g., one or more of insertions, deletions, gene fusions, inversions, etc.), a measure of genomic methylation, etc.). Exemplary methods for determining HRD score are described for example in Lotan, T. L., et. al. Mod Pathol 34, 1185-1193 (2021), which is hereby incorporated by reference in its entirety.

In some embodiments, HRD score may be determined using one or more features associated with homologous recombination deficiency. In some embodiments, such features comprise one or more of a mutation score associated with the cancer, a telomeric allelic imbalance score, a large-scale state transition (LST) score associated with the cancer, a loss of heterozygosity (LOH) score, a fraction of the genome having lost heterogeneity (fLOH), a homologous recombination deficiency score (the sum of one or more of the telomeric allelic imbalance (NtAI) score, a large-scale state transition (LST) score, a fraction of genome having lost heterogeneity (fLOH) and/or the loss of heterozygosity (LOH) score). In some embodiments, the homologous recombination deficiency (HRD) score is a sum of NtAI, LST and LOH.

The number of telomeric allelic imbalances (NtAI) score relates the number of subtelomeric regions with allelic imbalance that start from beyond the centromere and extend to the telomere. The largest-scale state transitions (LST) score relates to the number of large chromosomal breaks between adjacent regions, generally of at least about 10 megabases (Mb), although the specific threshold size may be increased or decreased. The loss of heterozygosity score (HRD-LOH) relates to the number of regions with a loss of heterozygosity, generally larger than 15 Mb (although the specific threshold size may be increased or decreased), but shorter than the whole chromosome. In some embodiments, the features may include one or more of NtAI, LST, LOH, or an HRD score (the sum of one or more of NtAI, LST and LOH).

In some embodiments, the one or more features comprise LST. In some embodiments, the one or more features do not include NtAI. In some embodiments, the one or more features do not include LOH. In some embodiments, the one or more features do not include NtAI and LOH. In some embodiments, HRD score, NtAI score, LST score and/or LOH score is determined by a microarray or by sequencing (for example, whole exome sequencing or whole genome sequencing) nucleic acids derived from the cancer. In some embodiments, fraction of the genome with a loss of heterogeneity (fLOH) may be used as a feature.

### Additional Biomarkers

In some embodiments, subjects have already been identified as having one or more additional biomarkers. In some embodiments, additional biomarkers are included in the selection criteria. In some embodiments, additional biomarkers are not included in the selection criteria. In other embodiments, the subject is selected determining the levels of other cancer biomarkers.

In some embodiments, subjects are selected based on CCNE1 gene amplification status, Cyclin E1 overexpression levels (or Cyclin E1 status) and HRD status.

In some embodiments, the subject is selected based on Cyclin E levels. In some embodiments the Cyclin E is Cyclin E1. In some embodiments, the CCNE1 levels are measured by a gene expression level. In some embodiments, the CCNE1 levels are compared to normal tissue to determine whether the expression level is an overexpression level. In some embodiments, the CCNE1 levels are measured by a gene overexpression level.

In some embodiments, the gene biomarker overexpression level is determined by detecting the amount of gene mRNA or protein. In some embodiments, the Cyclin E1 overexpression is determined by mRNA levels. In some embodiments, the Cyclin E1 overexpression is determined by protein levels. In some embodiments, the Cyclin E1 overexpression level is determined by a Cyclin E1 H-score.

Cyclin E1 mRNA or protein can be measured my methods known in the art including but not limited to an immunohistochemistry (IHC) test, reporter gene, Northern blot, Western blot, Fluorescent in situ hybridization (FISH), Reverse transcription PCR, or RNA-Seq based assays.

In some embodiments, Cyclin E1 overexpression is determined using RNA detection of cyclin E. In some embodiments, Cyclin E1 overexpression is determined using an RNA sequencing method.

In some embodiments, Cyclin E1 overexpression is measured by a quantitative readout. In some embodiments, Cyclin E1 overexpression is measured by a qualitative readout.

In some embodiments, Cyclin E1 overexpression is measured by signal intensity. In some embodiments, signal intensity is determined using a Western blot. In some embodiments, relative signal intensity is quantified.

In some embodiments, the subject is selected to have a TP53 biomarker level below a predetermined threshold. In some embodiments, the subject is selected to have a TP53 biomarker level above a predetermined threshold.

In some embodiments, the subject is selected to have a CA125 biomarker level below a predetermined threshold. In some embodiments, the subject is selected to have a CA125 biomarker level above a predetermined threshold.

### Methods of Treatment

The present disclosure provides methods of treating cancer using the compound known as azenosertib, or a pharmaceutically acceptable salt thereof, wherein subjects are selected to have an HRD positive status (e.g., a gene alteration in one or more HRRm genes). azenosertib (and pharmaceutically acceptable salts) is a WEE1 inhibitor of the formula:

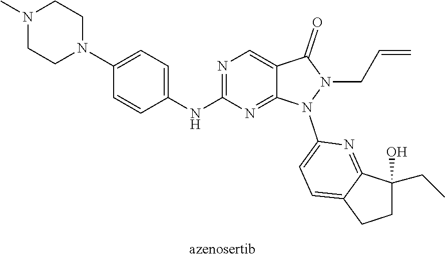 [SMILES: C=CCn1c(=O)c2cnc(Nc3ccc(N4CCN(C)CC4)cc3)nc2n1-c1ccc2c(n1)[C@@](O)(CC)CC2]

WO 2019/173082 and WO 2021/231653 describe the compound azenosertib, both of which are hereby incorporated by reference in their entireties.

In embodiments, a method described herein results in a therapeutic effect (e.g., a desired pharmacologic and/or physiologic effect). A therapeutic effect can encompass partially or completely curing a disease, relieving one or more adverse symptoms attributable to the disease and/or delaying progression of the disease. To this end, the method comprises administering an effective dose of a therapeutic agent (e.g., azenosertib, or a pharmaceutically acceptable salt thereof, and/or a second chemotherapeutic agent (including pharmaceutically acceptable salts of any of the foregoing)). An effective dose can be an amount effective, at dosages and for periods of time necessary, to achieve a desired therapeutic result (e.g., tumor growth inhibition, progression free survival, complete response, partial response etc.). An effective dose may vary according to factors such as the disease state, age, sex and weight of the individual, and the ability of the binding agent to elicit a desired response in the individual.

In some embodiments, therapeutic response may be determined according to Response Evaluation Criteria in Solid Tumors (RECIST) criteria.

In some embodiments, the treatment results in a response rate at or greater than 50%. In some embodiments, the response rate is measured by complete response (CR), partial response (PR), CA-125 50% response, or combination thereof.

In some embodiments, the treatment results in progression-free survival (PFS) of 6 months or longer.

In some embodiments, the inhibition of cancer is measured by inhibition of tumor growth. In some embodiments, the inhibition of tumor growth is measured by reduction of tumor volume.

### Administration Routes

In some embodiments, the effective dose of azenosertib, or a pharmaceutically acceptable salt thereof, is administered orally, intravenously or subcutaneously. In some embodiments, the effective dose of azenosertib, or a pharmaceutically acceptable salt thereof, is administered orally. One may also use alternative suitable techniques of administering the effective dose of azenosertib, or a pharmaceutically acceptable salt thereof, that are known to those skilled in the art including, but not limited to, oral, rectal, pulmonary topical, aerosol, injection, infusion and parenteral delivery, including intramuscular, subcutaneous, intravenous, intramedullary injections, intrathecal, direct intraventricular, intraperitoneal, intranasal and intraocular injections. In other embodiments, azenosertib, or a pharmaceutically acceptable salt thereof, and/or chemotherapeutic (including pharmaceutically acceptable salts thereof), can be administered orally.

In some embodiments, the effective dose of azenosertib, or a pharmaceutically acceptable salt thereof, is administered orally, intravenously, subcutaneously, intrathecally, intramuscularly, intracavitary, intrapleurally, intralesionally or intra-arterially. In some embodiments, the effective dose of azenosertib, or a pharmaceutically acceptable salt thereof, is administered orally, intravenously or subcutaneously. In some embodiments, the effective dose of azenosertib, or a pharmaceutically acceptable salt thereof, is administered intrathecally, intramuscularly, intracavitary, intrapleurally, intralesionally or intra-arterially.

In some embodiments, the effective dose of azenosertib, or a pharmaceutically acceptable salt thereof, is administered orally.

### Azenosertib Dosage and Schedule

In some embodiments, methods described herein comprises intermittent dosing, i.e., comprises consecutive days of dosing followed by break days, in one or more dosing cycles comprising intervening break weeks. In some embodiments, methods described herein comprise continuous dosing. In some embodiments, methods described herein comprise combination therapy comprises continuous dosing of one of the agents. In some embodiments, methods described herein comprise combination therapy comprises continuous dosing of one of the agents and intermittent dosing of azenosertib, or a pharmaceutically acceptable salt thereof.

In some embodiments, the effective dose of azenosertib, or a pharmaceutically acceptable salt thereof, is administered based on body weight of the subject. In some embodiments, the effective dose of azenosertib, or a pharmaceutically acceptable salt thereof, is between about 2 mg/kg and about 20 mg/kg. In some embodiments, the effective dose of azenosertib, or a pharmaceutically acceptable salt thereof, is between about 2 to about 18 mg/kg, about 2 to about 16 mg/kg, about 2 to about 14 mg/kg, about 2 to about 12 mg/kg, about 2 to about 10 mg/kg, about 2 to about 8 mg/kg, about 2 to about 6 mg/kg, about 3 to about 4 mg/kg, about 3 to about 5 mg/kg or about 4 to about 6 mg/kg. In some embodiments, the effective dose of azenosertib, or a pharmaceutically acceptable salt thereof, is at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg or at least about 19 mg/kg.

In some embodiments, azenosertib, or a pharmaceutically acceptable salt thereof, may also be in the form of equivalent dose (e.g., the compound in other salt forms). In some, embodiments, the effective dose is a flat dose. In some embodiments, the effective dose of azenosertib, or a pharmaceutically acceptable salt thereof, ranges from about 200 to about 800 mg/day, or equivalents thereof, once a day. In some embodiments, the effective dose of azenosertib, or a pharmaceutically acceptable salt thereof, ranges from about 200 to about 600 mg/day, or equivalents thereof, once a day. In some embodiments, the effective dose of azenosertib, or a pharmaceutically acceptable salt thereof, ranges from about 300 to about 600 mg/day, or equivalents thereof, once a day. In some embodiments, the effective dose of azenosertib, or a pharmaceutically acceptable salt thereof, ranges from about 400 to about 600 mg/day, or equivalents thereof, once a day. In some embodiments, the effective dose of azenosertib, or a pharmaceutically acceptable salt thereof, ranges from about 400 to about 800 mg/day, or equivalents thereof, once a day. In some embodiments, the effective dose of azenosertib, or a pharmaceutically acceptable salt thereof, ranges from about 50 to about 350 mg/day, about 50 to about 290 mg/day, about 100 to about 290 mg/day, about 100 to about 250 mg/day, about 150 to about 250 mg/day or about 180 to about 220 mg/day once a day, or equivalents thereof. In some embodiments, the effective dose of azenosertib, or a pharmaceutically acceptable salt thereof, ranges from about 50 to about 400 mg/day, about 100 to about 400 mg/day, about 150 to about 400 mg/day, about 200 to about 400 mg/day, about 200 to about 375 mg/day, about 200 to about 350 mg/day, about 200 to about 300 mg/day, about 200 to about 400 mg/day or about 400 to about 600 mg/day, or equivalents thereof, once a day.

In some embodiments, the effective dose of azenosertib, or a pharmaceutically acceptable salt thereof, is about 50 mg/day, about 100 mg/day, about 150 mg/day, about 200 mg/day, about 250 mg/day, about 300 mg/day, about 325 mg/day, about 350 mg/day, about 375 mg/day, about 400 mg/day, about 450 mg/day, about 500 mg/day, about 550 mg/day or about 600 mg/day, or equivalents thereof, once a day.

In some embodiments, the effective dose of azenosertib, or a pharmaceutically acceptable salt thereof, is about 200 mg/day, or equivalents thereof, once a day. In some embodiments, the effective dose of azenosertib, or a pharmaceutically acceptable salt thereof, is about 300 mg/day, or equivalents thereof, once a day. In some embodiments, the effective dose of azenosertib, or a pharmaceutically acceptable salt thereof, is about 400 mg/day, or equivalents thereof, once a day. In some embodiments, the effective dose of azenosertib, or a pharmaceutically acceptable salt thereof, is about 500 mg/day, or equivalents thereof, once a day. In some embodiments, the effective dose of azenosertib, or a pharmaceutically acceptable salt thereof, is about 600 mg/day, or equivalents thereof, once a day. In some embodiments, the effective dose of azenosertib, or a pharmaceutically acceptable salt thereof, is about 700 mg/day, or equivalents thereof, once a day. In some embodiments, the effective dose of azenosertib, or a pharmaceutically acceptable salt thereof, is about 800 mg/day, or equivalents thereof, once a day.

In some embodiments, the effective dose of azenosertib, or a pharmaceutically acceptable salt thereof, is at or greater than about 375 mg/day, about 400 mg/day, about 425 mg/day, about 450 mg/day, about 475 mg/day, about 500 mg/day, about 550 mg/day, about 600 mg/day, about 625 mg/day, about 650 mg/day, about 675 mg/day, about 700 mg/day, about 725 mg/day, about 750 mg/day, about 775 mg/day or about 800 mg/day, or an equivalent thereof. In some embodiments, the present disclosure provides administration of a high dose of azenosertib, or a pharmaceutically acceptable salt thereof, e.g., wherein the dose is or greater than about 375 mg/day.

In some embodiments, the effective dose of azenosertib, or a pharmaceutically acceptable salt thereof, is at least about 250 mg/day. In some embodiments, the effective dose of azenosertib, or a pharmaceutically acceptable salt thereof, is about 300 mg/day. In some embodiments, the dose of azenosertib, or a pharmaceutically acceptable salt thereof, is about 350 mg/day. In some embodiments, the dose of azenosertib, or a pharmaceutically acceptable salt thereof, is about 400 mg/day.

In some embodiments, the effective dose of azenosertib, or a pharmaceutically acceptable salt thereof, is between about 250 to about 450 mg/day. In some embodiments, the effective dose of azenosertib, or a pharmaceutically acceptable salt thereof, is at least about 300 mg/day. In some embodiments, the effective dose of azenosertib, or a pharmaceutically acceptable salt thereof, is about 300 mg. In some embodiments, the effective dose of azenosertib, or a pharmaceutically acceptable salt thereof, is about 400 mg/day.

In some embodiments, the effective dose of azenosertib, or a pharmaceutically acceptable salt thereof, is between about 60 to about 120 mg/kg. In some embodiments, the effective dose of azenosertib, or a pharmaceutically acceptable salt thereof, is about 60 mg/kg. In some embodiments, the effective dose of azenosertib, or a pharmaceutically acceptable salt thereof, is about 80 mg/kg. In some embodiments, the effective dose of azenosertib, or a pharmaceutically acceptable salt thereof, is about 100 mg/kg. In some embodiments, the effective dose of azenosertib, or a pharmaceutically acceptable salt thereof, is about 120 mg/kg.

In some embodiments, the cancer is ovarian cancer, and the effective dose of azenosertib, or a pharmaceutically acceptable salt thereof, is one or more cycles of 5:2 (5 with dosing: 2 without dosing) at about 60 mg/kg. In some embodiments, the cancer is ovarian cancer, and the effective dose of azenosertib, or a pharmaceutically acceptable salt thereof, is one or more cycles of 5:2 (5 with dosing: 2 without dosing) at about 80 mg/kg. In some embodiments, the cancer is ovarian cancer, the HRD is a BRCA1 mutation and the effective dose of azenosertib, or a pharmaceutically acceptable salt thereof, is at least about 60 mg/kg. In some embodiments, the cancer is ovarian cancer, the HRD is a BRCA1 mutation and the effective dose of azenosertib, or a pharmaceutically acceptable salt thereof, is at least about 80 mg/kg.

In some embodiments, the cancer is prostate cancer, and the effective dose of azenosertib, or a pharmaceutically acceptable salt thereof, is one or more cycles of 5:2 (5 with dosing: 2 without dosing) at about 60 mg/kg. In some embodiments, the cancer is prostate cancer, and the effective dose of azenosertib, or a pharmaceutically acceptable salt thereof, is one or more cycles of 5:2 (5 with dosing: 2 without dosing) at about 80 mg/kg. In some embodiments, the cancer is prostate cancer, the HRD is a BRCA1 mutation and the effective dose of azenosertib, or a pharmaceutically acceptable salt thereof, is at least about 60 mg/kg. In some embodiments, the cancer is prostate cancer, the HRD is a BRCA1 mutation and the effective dose of azenosertib, or a pharmaceutically acceptable salt thereof, is at least about 80 mg/kg.

### Treatment Cycle

Methods of the present disclosure include administering azenosertib, or a pharmaceutically acceptable salt thereof, in a suitable dosing schedule. In some embodiments, the effective dose is administered once a day. In some embodiments, the effective dose is administered twice a day. In some embodiments, the effective dose is administered three times a day. In some embodiments, the effective dose is administered on a continuous dosing schedule. In some embodiments, the effective dose is administered on an intermittent dosing schedule. In some embodiments, the intermittent dosing cycle comprises five days with dosing (e.g., five consecutive days with dosing) and two days without dosing.

In some embodiments, azenosertib, or a pharmaceutically acceptable salt thereof, is administered in combination with a second chemotherapeutic agent, or a pharmaceutically acceptable salt thereof, in a suitable dosing schedule. For example, the azenosertib, or a pharmaceutically acceptable salt thereof, and/or a second chemotherapeutic agent, or a pharmaceutically acceptable salt thereof, described herein may be administered one or more times per day (for example once, twice or three times a day) for a certain number of days, followed by a period of days where no dose is given. This treatment cycle (including days with dosing and no-days with dosing) may then be repeated.

In some embodiments, a treatment cycle is a period of 3-28 days. In some embodiments, a treatment cycle is 5, 7, 10 or 14 days. In some embodiments, the treatment cycle is 21 days or 28 days. In some embodiments, the treatment cycle is repeated.

In some aspects, provided herein is a method of treating cancer comprising administering to a subject in need thereof, an effective dose of azenosertib, or a pharmaceutically acceptable salt thereof, at or greater than about 350 mg/day, or an equivalent thereof, in accordance with an intermittent dosing cycle, wherein the intermittent dosing cycle comprises one or more dosing weeks with each dosing week comprising at least three consecutive days with dosing with dosing and at least one day without dosing.

In some aspects, the present disclosure provides administration of a high dose of azenosertib, or a pharmaceutically acceptable salt thereof, e.g., between about 350 mg to about 800 mg once a day, or between about 175 mg to about 400 mg twice a day at an intermittent dosing regimen, e.g., 5 days administration (“on” days) followed by 2 days break (“off” days) i.e., 5/2, 4 days administration followed by 3 days break i.e., 4/3, or 3 days administration followed by 4 days off, i.e., 3/4, or 6 days administration followed by 1 day off i.e., 6/1. Alternatively, the intermittent dosing regimen of azenosertib, or a pharmaceutically acceptable salt thereof, is also expressed as administering between about 350 mg to about 800 mg once a day, or between about 175 mg to about 400 mg twice a day at an intermittent frequency, e.g., 5 on/2 off, 4 on/2 off, 3 on/4 off, among others. In some embodiments, the intermittent dosing cycle comprises five consecutive days with dosing and two days without dosing.

In some embodiments, the one or more dosing weeks are separated by at least one week of break. In some embodiments, the intermittent dosing regimen described herein (for example, of 7/0, 6/1, 5/2, 4/3 or 3/4) is carried out for 2 weeks followed by one week of break, or one week followed by one week of break, thereby achieving a high efficacy while increasing safety and tolerability in treating a cancer. In some embodiments, the intermittent dosing regimen described herein (for example, of 7/0, 5/2, 6/1, 4/3 or 3/4) is carried out for 3 weeks followed by one week of break, or one week followed by one week of break, thereby achieving a high efficacy while increasing safety and tolerability in treating a cancer. In some embodiments, the intermittent dosing regimen described herein (for example, of 7/0, 6/1, 5/2, 4/3 or 3/4) is carried out for greater than 3 weeks followed by one week of break, or one week followed by one week of break, thereby achieving a high efficacy while increasing safety and tolerability in treating a cancer.

In some aspects, provided herein is a method of treating cancer comprising: administering to a subject in need thereof, an effective dose of azenosertib, or a pharmaceutically acceptable salt thereof, at or greater than about 100 mg/day, or an equivalent thereof, in accordance with an intermittent dosing cycle, wherein the intermittent dosing cycle comprises one or more dosing weeks with each dosing week comprises at least three consecutive days with dosing and at least one day without dosing, followed by at least one week of break. In some embodiments, the effective dose of azenosertib, or a pharmaceutically acceptable salt thereof, is at or greater than about 100 mg/day, about 125 mg/day, about 150 mg/day, about 175 mg/day, about 200 mg/day, about 225 mg/day, about 250 mg/day, about 275 mg/day, about 300 mg/day, about 325 mg/day or about 350 mg/day, or an equivalent thereof. In some embodiments, azenosertib, or a pharmaceutically acceptable salt thereof, is administered at an effective dose of about 200 mg once a day in an intermittent dosing regimen. In some embodiments, azenosertib, or a pharmaceutically acceptable salt thereof, is administered at an effective dose of about 225 mg once a day in an intermittent dosing regimen. In some embodiments, azenosertib, or a pharmaceutically acceptable salt thereof, is administered at an effective dose of about 250 mg once a day in an intermittent dosing regimen. In some embodiments, azenosertib, or a pharmaceutically acceptable salt thereof, is administered at an effective dose of about 275 mg once a day in an intermittent dosing regimen. In some embodiments, azenosertib, or a pharmaceutically acceptable salt thereof, is administered at an effective dose of greater than about 300 mg once a day in an intermittent dosing regimen. In some embodiments, azenosertib, or a pharmaceutically acceptable salt thereof, is administered at an effective dose of about 300 mg once a day in an intermittent dosing regimen. In some embodiments, azenosertib, or a pharmaceutically acceptable salt thereof, is administered at an effective dose of about 350 mg once a day in an intermittent dosing regimen.

In some embodiments, the effective dose of azenosertib, or a pharmaceutically acceptable salt thereof, is at or greater than about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 550 mg/day, about 600 mg/day, about 625 mg/day, about 650 mg/day, about 675 mg/day, about 700 mg/day, about 725 mg/day, about 750 mg/day, about 775 mg/day or about 800 mg/day, or an equivalent thereof. In some embodiments, the effective dose of azenosertib, or a pharmaceutically acceptable salt thereof, is at or greater than about 250 mg/day. In some embodiments, the effective dose of azenosertib, or a pharmaceutically acceptable salt thereof, is at or greater than about 275 mg/day. In some embodiments, the effective dose of azenosertib, or a pharmaceutically acceptable salt thereof, is at or greater than about 300 mg/day. In some embodiments, the effective dose of azenosertib, or a pharmaceutically acceptable salt thereof, is at or greater than about 325 mg/day. In some embodiments, the effective dose of azenosertib, or a pharmaceutically acceptable salt thereof, is at or greater than about 350 mg/day. In some embodiments, the effective dose of azenosertib, or a pharmaceutically acceptable salt thereof, is at or greater than about 375 mg/day. In some embodiments, the effective dose of azenosertib, or a pharmaceutically acceptable salt thereof, is at about 400 mg/day. In some embodiments, the effective dose of azenosertib, or a pharmaceutically acceptable salt thereof, is at about 425 mg/day. In some embodiments, the effective dose of azenosertib, or a pharmaceutically acceptable salt thereof, is at about 450 mg/day. In some embodiments, the effective dose of azenosertib, or a pharmaceutically acceptable salt thereof, is at about 475 mg/day. In some embodiments, the effective dose of azenosertib, or a pharmaceutically acceptable salt thereof, is at about 500 mg/day. In some embodiments, the effective dose of azenosertib, or a pharmaceutically acceptable salt thereof, is at about 550 mg/day. In some embodiments, the effective dose of azenosertib, or a pharmaceutically acceptable salt thereof, is at about 600 mg/day. In some embodiments, the effective dose of azenosertib, or a pharmaceutically acceptable salt thereof, is at about 625 mg/day. In some embodiments, the effective dose of azenosertib, or a pharmaceutically acceptable salt thereof, is at about 650 mg/day. In some embodiments, the effective dose of azenosertib, or a pharmaceutically acceptable salt thereof, is at about 675 mg/day. In some embodiments, the effective dose of azenosertib, or a pharmaceutically acceptable salt thereof, is at about 700 mg/day. In some embodiments, the effective dose of azenosertib, or a pharmaceutically acceptable salt thereof, is at about 725 mg/day. In some embodiments, the effective dose of azenosertib, or a pharmaceutically acceptable salt thereof, is at about 750 mg/day. In some embodiments, the effective dose of azenosertib, or a pharmaceutically acceptable salt thereof, is at about 775 mg/day. In some embodiments, the effective dose of azenosertib, or a pharmaceutically acceptable salt thereof, is at about 800 mg/day, or an equivalent thereof.

In some embodiments, the effective dose of azenosertib, or a pharmaceutically acceptable salt thereof, is between about 250 to about 450 mg/day.

In some embodiments, the effective dose of azenosertib, or a pharmaceutically acceptable salt thereof, is at about 300 mg/day.

In some embodiments, the effective dose of azenosertib, or a pharmaceutically acceptable salt thereof, is at about 350 mg/day.

In some embodiments, the effective dose of azenosertib, or a pharmaceutically acceptable salt thereof, is at about 400 mg/day.

In some embodiments, the effective dose of azenosertib, or a pharmaceutically acceptable salt thereof, is administered once per day.

In some embodiments, the effective dose of azenosertib, or a pharmaceutically acceptable salt thereof, is divided into twice per day.

In some embodiments, the effective dose of azenosertib, or a pharmaceutically acceptable salt thereof, is divided into three times per day.

In some embodiments, each dosing week comprises at least four, five or six consecutive days with dosing.

In some embodiments, each dosing week comprises five consecutive days with dosing and two days without dosing.

In some embodiments, each dosing week comprises four consecutive days with dosing and three days without dosing.

In some embodiments, each dosing week comprises three consecutive days with dosing and four days without dosing.

In some embodiments, each dosing week comprises seven consecutive days with dosing and seven days without dosing.

In some embodiments, each intermittent dosing cycle comprises between about 7 days to about 10 consecutive days with dosing. In some embodiments, each intermittent dosing cycle comprises between about 8 consecutive days with dosing. In some embodiments, each intermittent dosing cycle comprises between about 9 consecutive days with dosing. In some embodiments, each intermittent dosing cycle comprises between about 10 consecutive days with dosing.

In some embodiments, the intermittent dosing cycle comprises twenty-one consecutive days with dosing and seven days without dosing.

In some embodiments, the intermittent dosing cycle comprises two consecutive dosing weeks.

In some aspects, provided herein is a method of treating cancer comprising administering to a subject in need thereof, an effective dose of azenosertib, or a pharmaceutically acceptable salt thereof, at or greater than about 350 mg/day, or an equivalent thereof, in accordance with an intermittent dosing cycle, wherein the intermittent dosing cycle comprises at least two consecutive days with dosing and at least one day without dosing.

In some embodiments, the intermittent dosing cycle comprises at least three, four, five, six, seven, eight, nine, ten, eleven, twelve, thirteen or fourteen consecutive days with dosing. In some embodiments, the intermittent dosing cycle comprises greater than fourteen consecutive days with dosing. In some embodiments, the intermittent dosing cycle comprises twenty-one consecutive days with dosing. In some embodiments, the intermittent dosing cycle comprises twenty-eight consecutive days with dosing. In some embodiments, the intermittent dosing cycle comprises thirty-two consecutive days with dosing. In some embodiments, the intermittent dosing cycle comprises forty-two consecutive days with dosing.

In some embodiments, the intermittent dosing cycle comprises at least one two, three, four, five, six, or seven days without dosing. In some embodiments, the intermittent dosing cycle comprises one day without dosing. In some embodiments, the intermittent dosing cycle comprises between about two to about seven days without dosing. In some embodiments, the intermittent dosing cycle comprises two days without dosing. In some embodiments, the intermittent dosing cycle comprises three days without dosing. In some embodiments, the intermittent dosing cycle comprises four days without dosing. In some embodiments, the intermittent dosing cycle comprises five days without dosing. In some embodiments, the intermittent dosing cycle comprises six days without dosing. In some embodiments, the intermittent dosing cycle comprises seven days with dosing.

In some embodiments, the intermittent dosing cycle includes consecutive days with dosing of between about two to about seven days (“on” days), followed by a period of break of between about one to about seven days (“off” days).

In some embodiments, the intermittent dosing cycle comprises five consecutive days with dosing and two days without dosing.

In some embodiments, the intermittent dosing cycle comprises four consecutive days with dosing and three days without dosing.

In some embodiments, the intermittent dosing cycle comprises three consecutive days with dosing and four days without dosing.

In some embodiments, the intermittent dosing cycle comprises six consecutive days with dosing and one day without dosing.

In some embodiments, the intermittent dosing cycle comprises seven consecutive days with dosing and seven days without dosing.

In some embodiments, the intermittent dosing cycle comprises fourteen consecutive days with dosing and seven days without dosing.

In some embodiments, the intermittent dosing cycle comprises twenty-one consecutive days with dosing and seven days without dosing.

In some embodiments, the effective dose of azenosertib, or a pharmaceutically acceptable salt thereof, is at or greater than about 375 mg/day, about 400 mg/day, about 425 mg/day, about 450 mg/day, about 475 mg/day, about 500 mg/day, about 550 mg/day, about 600 mg/day, about 625 mg/day, about 650 mg/day, about 675 mg/day, about 700 mg/day, about 725 mg/day, about 750 mg/day, about 775 mg/day or about 800 mg/day, or an equivalent thereof.

In some embodiments, azenosertib, or a pharmaceutically acceptable salt thereof, is administered at a dose of about 300 mg once a day in an intermittent dosing regimen. In some embodiments, azenosertib, or a pharmaceutically acceptable salt thereof, is administered at a dose of about 350 mg once a day in an intermittent dosing regimen. In some embodiments, azenosertib, or a pharmaceutically acceptable salt thereof, is administered at a dose of about 400 mg once a day in an intermittent dosing regimen.

In some embodiments, azenosertib, or a pharmaceutically acceptable salt thereof, is administered at a dose of at least about 300 mg once a day in an intermittent dosing regimen. In some embodiments, azenosertib, or a pharmaceutically acceptable salt thereof, is administered at a dose of at least about 350 mg once a day in an intermittent dosing regimen. In some embodiments, azenosertib, or a pharmaceutically acceptable salt thereof, is administered at a dose of at least about 400 mg once a day in an intermittent dosing regimen.

In some embodiments, the present disclosure provides administration of a high dose of azenosertib, or a pharmaceutically acceptable salt thereof, e.g., wherein the dose is or greater than about 375 mg. In some embodiments, azenosertib, or a pharmaceutically acceptable salt thereof, is administered at a dose of about 400 mg once a day in an intermittent dosing regimen. In some embodiments, azenosertib, or a pharmaceutically acceptable salt thereof, is administered at a dose of about 450 mg once a day in an intermittent dosing regimen. In some embodiments, azenosertib, or a pharmaceutically acceptable salt thereof, is administered at a dose of about 500 mg once a day in an intermittent dosing regimen. In some embodiments, azenosertib, or a pharmaceutically acceptable salt thereof, is administered at a dose of about 550 mg once a day in an intermittent dosing regimen. In some embodiments, azenosertib, or a pharmaceutically acceptable salt thereof, is administered at a dose of about 600 mg once a day in an intermittent dosing regimen. In some embodiments, azenosertib, or a pharmaceutically acceptable salt thereof, is administered at a dose of about 625 mg once a day in an intermittent dosing regimen. In some embodiments, azenosertib, or a pharmaceutically acceptable salt thereof, is administered at a dose of about 650 mg once a day in an intermittent dosing regimen. In some embodiments, azenosertib, or a pharmaceutically acceptable salt thereof, is administered at a dose of about 700 mg once a day in an intermittent dosing regimen. In some embodiments, azenosertib, or a pharmaceutically acceptable salt thereof, is administered at a dose of about 750 mg once a day in an intermittent dosing regimen. In some embodiments, azenosertib, or a pharmaceutically acceptable salt thereof, is administered at a dose of about 775 mg once a day in an intermittent dosing regimen. In some embodiments, azenosertib, or a pharmaceutically acceptable salt thereof, is administered at a dose of about 800 mg once a day in an intermittent dosing regimen.

In some embodiments, the effective dose of azenosertib, or a pharmaceutically acceptable salt thereof, is administered once per day.

In some embodiments, the effective dose of azenosertib, or a pharmaceutically acceptable salt thereof, is divided equally into twice per day.

In some embodiments, the effective dose of azenosertib, or a pharmaceutically acceptable salt thereof, is divided equally into three doses per day. In some embodiments, the effective dose of azenosertib, or a pharmaceutically acceptable salt thereof, is divided equally into four doses per day.

In some embodiments, the twice per day of azenosertib, or a pharmaceutically acceptable salt thereof, is at or greater than about 175 mg, about 200 mg, about 225 mg, about 250 mg, about 275 mg, about 300 mg, about 325 mg, about 350 mg, about 375 mg or about 400 mg, or an equivalent thereof.

In some embodiments, the intermittent dosing cycle is repeated. In some aspects, provided herein is a method of treating cancer comprising administering to a subject in need thereof, an effective dose of azenosertib, or a pharmaceutically acceptable salt thereof, at or greater than about 400 mg/day, or an equivalent thereof, in accordance with an intermittent dosing cycle, wherein the intermittent dosing cycle comprises at five consecutive days with dosing and two days without dosing.

In some embodiments, the method further comprises administering a second chemotherapeutic agent during the intermittent dosing cycle. Without wishing to be bound by any particular theory, administration of azenosertib, or a pharmaceutically acceptable salt thereof, in combination with a second chemotherapeutic agent renders responsive a subject resistant to treatment by the second chemotherapeutic agent, or a pharmaceutically acceptable salt thereof, alone, or prevents or reduces toxicity by the agent and/or improves efficacy of treatment as compared to monotherapy. Combination therapy using intermittent dosing cycle further benefits dosing by requiring, for example, a lower effective dose of the second chemotherapeutic agent, or a pharmaceutically acceptable salt thereof, and/or azenosertib (including pharmaceutically acceptable salts thereof).

In some embodiments, azenosertib, or a pharmaceutically acceptable salt thereof, is administered in combination with one or more second chemotherapeutic agents (including pharmaceutically acceptable salts thereof) in an intermittent dosing cycle.

### Cancer Types

Methods of the present disclosure can be used to treat cancers.

As described herein, the cancer is associated with a “homologous recombination deficiency,” “homologous recombination repair deficiency”, “homologous repair deficiency,” “HDI,” “HRD-positive,” or “HRD+” which refers to a reduction or impairment of the homologous recombination process.

In some embodiments, the cancer is also associated with increased CCNE1 gene amplification and/or Cyclin E1 expression levels (e.g., a cyclin E amplified cancer, a cyclin E overexpressing/not amplified cancer, a cyclin E driven cancer).

In some embodiments, the cancer is glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colon cancer, colorectal cancer, endometrial cancer, endometrium cancer, esophagus cancer, eye cancer, gallbladder cancer, gastric cancer, gastrointestinal cancer, Hodgkin lymphoma, Non-Hodgkin lymphoma, hematological tumor, head cancer, heme malignancy, Kaposi sarcoma, kidney cancer, laryngeal and hypopharyngeal cancer, liver cancer, lung cancer, non-small cell lung cancer (NSCLC), small cell, lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, uterine sarcoma, uterine serous carcinoma (USC), uterine CS, vaginal cancer, vulvar cancer, Waldenstrom macroglobulinemia, Wilms tumor, solid tumor, or liquid tumor, HGSOC, invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymoma, BRAF mutant metastatic colorectal cancer, or uveal melanoma.

In some embodiments, the subject has a cancer. In some embodiments, the cancer is breast cancer, brain cancer, lung cancer, liver cancer, stomach cancer, spleen cancer, colon cancer, renal cancer, pancreatic cancer, prostate cancer, uterine cancer, skin cancer, head cancer, neck cancer, sarcomas, neuroblastomas or ovarian cancer.

In some embodiments, the cancer is glioblastoma, astrocytoma, meningioma, craniopharyngioma, medulloblastoma, other brain cancers, leukemia, skin cancer, adrenal cancer, anal cancer, bile duct cancer, bladder cancer, bone cancer, breast cancer, cervical cancer, colorectal cancer, endometrial cancer, esophagus cancer, eye cancer, gallbladder cancer, gastrointestinal cancer, Hodgkin lymphoma, hematological tumor, heme malignancy, Kaposi sarcoma, kidney cancer, laryngeal and hypopharyngeal cancer, liver cancer, lung cancer, lymphoma, mesothelioma, melanoma, multiple myeloma, neuroblastoma, nasopharyngeal cancer, ovarian cancer, osteosarcoma, pancreatic cancer, pituitary cancer, retinoblastoma, salivary gland cancer, stomach cancer, small intestine cancer, testicular cancer, thymus cancer, thyroid cancer, uterine cancer, uterine sarcoma, uterine serous carcinoma (USC), vaginal cancer, vulvar cancer, Waldenstrom macroglobulinemia, Wilms tumor, solid tumor or liquid tumor.

In some embodiments, the cancer is a solid tumor or a heme malignancy. In some embodiments, the cancer is a solid tumor. In some embodiments, the solid tumor is selected from endometrial cancer, gallbladder cancer, ovarian cancer, HGSOC, endometrium cancer, melanoma, colorectal cancer, bladder cancer, breast cancer, invasive breast cancer, Triple Negative Breast Cancer (TNBC), prostate cancer, Lung cancer, NSCLC, SCLC esophagogastric cancer, gastric cancer, esophageal cancer, renal cancer, pRCC, ccRCC, chromophobe RCC, head and neck cancer, osteosarcoma cancer, pancreatic cancer, brain cancer, uterine CS, uterine cancer, adenoid cystic carcinoma (ACC), mesothelioma, cervical cancer, Diffuse large B cell lymphoma (DLBCL), non-Hodgkin lymphoma (NHL), liver cancer, glioblastoma (GBM), testicular cancer, Low-grade gliomas (LGGs), Pheochromocytoma and paraganglioma (PCPGs), cholangiocarcinoma, thyroid cancer, thymoma and uveal melanoma.

In some embodiments, the solid tumor is ovarian cancer. In some embodiments, the ovarian cancer is epithelial ovarian cancer, germ cell cancer or stromal cancer. In some embodiments, the ovarian cancer is epithelial ovarian cancer. In some embodiments, the epithelial ovarian cancer is high grade serous ovarian cancer (HGSOC).

In some embodiments, the cancer is associated with an organ selected from adrenal gland, ampulla of vater, biliary tract, bladder/urinary tract, bone, bowel, breast, cervix, CNS/brain, esophagus/stomach, eye, head and neck, kidney, liver, lung, lymphoid, myeloid, ovary/fallopian tube, pancreas, penis, peripheral nervous system, peritoneum, pleura, prostate, skin, soft tissue, testis, thymus, thyroid, uterus, vulva/vagina, adenocarcinoma in situ, extra gonadal germ cell tumor (EGCT), a mixed cancer type, high-grade neuroendocrine carcinoma of the ovary, high-grade serous fallopian tube cancer (HGSFT), ovarian choriocarcinoma and NOS (OCNOS).

In some embodiments, the cancer is a primary cancer that originates in the associated organ. In some embodiments, the cancer is primary peritoneal cancer.

In some embodiments, the cancer has metastasized to the associated organ.

In some embodiments, the cancer is a solid tumor or a heme malignancy.

In some embodiments, the cancer is a solid tumor.

In some embodiments, the solid tumor the solid tumor is selected from endometrial cancer, gallbladder cancer, ovarian cancer (e.g., HGSOC), endometrium cancer, melanoma, colorectal cancer, bladder cancer, breast cancer (e.g., invasive, Triple negative breast cancer (TNBC)), prostate cancer, Lung cancer (e.g., NSCLC, SCLC), esophagogastric cancer, gastric cancer, esophageal cancer, renal cancer (e.g., pRCC, ccRCC, chromophobe RCC), head and neck cancer, osteosarcoma cancer, pancreatic cancer, brain cancer, uterine CS, uterine cancer, adenoid cystic carcinoma (ACC), mesothelioma, cervical cancer, Diffuse large B cell lymphoma (DLBCL), non-Hodgkin lymphoma (NHL), liver cancer, glioblastoma (GBM), testicular cancer, Low-grade gliomas (LGGs), Pheochromocytoma and paraganglioma (PCPGs), cholangiocarcinoma, thyroid cancer, thymoma and uveal melanoma.

In some embodiments, the cancer is acute myeloid leukemia (AMIL).

In some embodiments, the tumor is neuroendocrine tumor, neuroendocrine prostate cancer or pancreatic neuroendocrine tumor.

In some embodiments, the solid tumor is ovarian cancer.

In some embodiments, the ovarian cancer is epithelial ovarian cancer, germ cell cancer, or stromal cancer.

In some embodiments, the ovarian cancer is epithelial ovarian cancer,

In some embodiments, the ovarian cancer is high grade serous ovarian cancer (HGSOC). In some embodiments, the ovarian cancer is platinum-resistant ovarian cancer (PROC). In some embodiments, the ovarian cancer is CCNE1-amplified ovarian cancer. In some embodiments, the ovarian cancer is a Cyclin E1 overexpressing cancer. In some embodiments, the ovarian cancer is a Cyclin E1 overexpressing/non-CCNE1-amplified cancer.

### PARP-Inhibitor Resistant Cancer

In some embodiments, the cancer is PARP inhibitor-resistant. PARP inhibitor resistance refers to non-responsiveness of the cancer to treatment with a PARP inhibitor (including pharmaceutically acceptable salts thereof). In some embodiments, the PARP inhibitor resistant cancer is intrinsically resistant to treatment with a PARP inhibitor (including pharmaceutically acceptable salts thereof). In some embodiments, the PARP inhibitor-resistant cancer has acquired resistance to treatment with a PARP inhibitor (including pharmaceutically acceptable salts thereof). For example, cancer cells with mutations in tumor suppressor genes cannot repair DNA through homologous recombination. PARP inhibitors cause DNA damage that can be repaired by normal cells, but not tumor cells; thus, PARP inhibitors selectively kill tumor cells, but not normal cells. In some embodiments, reversion mutations occur in tumor suppressor genes (e.g., BRCA reversion mutations) causing tumor cells to survive after PARP inhibitor treatment. In some embodiments, PARP inhibitor resistance is due to changes to other genes such as TP53BP1.

In some embodiments, the PARP inhibitor is selected from the group consisting of olaparib, niraparib, rucaparib, talazoparib, veliparib, pamiparib (BGB-290), iniparib (BSI 201), saruparib (AZD-5305), E7016 (Esai) and CEP-9722, or a pharmaceutically acceptable salt of any of the foregoing.

In some embodiments, the cancer is olaparib-resistant. In some embodiments, the cancer is niraparib-resistant. In some embodiments, the cancer is saruparib-resistant. In some embodiments, the cancer is rucaparib-resistant. In some embodiments, the cancer is talazoparib-resistant. In some embodiments, the cancer is veliparib-resistant. In some embodiments, the cancer is pamiparib-resistant. In some embodiments, the cancer is iniparib-resistant. In some embodiments, the cancer is E7016-resistant. In some embodiments, the cancer is CEP-9722-resistant. In some embodiments, provided herein is a method of treating PARP inhibitor-resistant breast cancer, comprising: determining or having determined whether a subject in need of treatment has a homologous recombination repair deficiency (HRD), and if the subject is HRD+, administering azenosertib, or a pharmaceutically acceptable salt thereof, at an effective dose, wherein the administration of azenosertib, or a pharmaceutically acceptable salt thereof, results in the inhibition of the PARP inhibitor-resistant breast cancer in the subject. In some embodiments, the PARP inhibitor-resistant breast cancer is niraparib-resistant. In some embodiments, the PARP inhibitor-resistance breast cancer is olaparib-resistant.

In some embodiments, the cancer is uterine serous carcinoma (USC).

In some embodiments, the cancer is osteosarcoma.

In some embodiments, the solid tumor is a uterine serous carcinoma, ovarian cancer, peritoneal cancer, fallopian tube cancer, osteosarcoma, pancreatic cancer or BRAF-mutant metastatic colorectal cancer.

In some embodiments, the cancer is acute myeloid leukemia (AML), acute lymphocytic leukemia (ALL), chronic myeloid leukemia (CMIL), chronic lymphocytic leukemia (CLL), chronic myelomonocytic leukemia (CMML), cutaneous B-cell lymphoma, cutaneous T-cell lymphoma, Hodgkin's lymphoma, Non-Hodgkin's lymphoma, Waldenstrom macroglobulinemia or multiple myeloma (MM).

### Chemotherapy Resistant Cancer

In some embodiments, the cancer is chemotherapy-resistant. In some embodiments, the cancer is resistant to one or more chemotherapy regimens. In some embodiments, the cancer is refractory to one or more chemotherapy regimens. In some embodiments, the chemotherapeutic agent is selected from bendamustine, bortezomib, carfilzomib, ixazomib, busulfan, carboplatin, cisplatin, cyclophosphamide, cladribine, paclitaxel, docetaxel, pegylated liposomal doxorubicin (PLD), dexamethasone, doxorubicin, gemcitabine, cytarabine, fludarabine, fluorouracil (5-FU), irinotecan, topotecan, temozolomide, triapine, azacitidine, 5-azacytidine, capecitabine, AraC-FdUMP[10](CF-10), cladribine, etoposide, decitabine, daunorubicin, doxorubicin, ifosfamide, methotrexate, vincristine, hydroxyurea and oxaliplatin, or a pharmaceutically acceptable salt of any of the foregoing.

In some embodiments, the cancer is a platinum-refractory cancer or a platinum-resistant cancer.

Platinum-resistant cancer is cancer that responds to treatment with drugs that contain the metal platinum, such as cisplatin and carboplatin, but recurs within a certain period, e.g., ovarian cancer that recurs after 6 months of remission. Platinum resistance, defined as the lack of response or relapse within six months of platinum-based chemotherapy, is a determinant of survival.

In some embodiments, the cancer is a platinum-refractory cancer. Platinum refractory cancer is a cancer that does not respond to treatment with anticancer drugs that contain the metal platinum, such as cisplatin and carboplatin.

In some aspects, provided herein is a method of treating chemotherapy-resistant ovarian cancer, comprising: determining whether a subject in need of treatment has a homologous recombination repair deficiency (HRD) or a HRD status, and if the subject has a HRD or has a HRD-positive (HRD+) status, administering azenosertib, or a pharmaceutically acceptable salt thereof, at an effective dose, wherein the administration of azenosertib, or a pharmaceutically acceptable salt thereof, results in the inhibition of the chemotherapy-resistant ovarian cancer in the subject. In some embodiments, the chemotherapy-resistant ovarian cancer is platinum resistant. In some embodiments, the chemotherapy-resistant ovarian cancer is further resistant to a PARP inhibitor, or a pharmaceutically acceptable salt thereof.

### Combination Therapies

The present disclosure provides methods of using azenosertib, or a pharmaceutically acceptable salt thereof, in combination with one or more additional agents (e.g., combination therapy with chemotherapeutic agents, including pharmaceutically acceptable salts thereof). In one aspect, the present disclosure provides a method of treating cancer comprising, administering to a subject selected to have a homologous recombination repair deficiency (HRD) biomarker, an effective dose of azenosertib, or a pharmaceutically acceptable salt thereof, and a second chemotherapeutic agent, or a pharmaceutically acceptable salt thereof.

Combination therapy refers to a clinical intervention in which a subject is simultaneously exposed to two or more therapeutic regimens (e.g., azenosertib, or a pharmaceutically acceptable salt thereof, and a second chemotherapeutic agent, or a pharmaceutically acceptable salt thereof). In some embodiments, the two or more chemotherapeutic regimens (including pharmaceutically salts thereof) may be administered simultaneously. In some embodiments, the two or more chemotherapeutic regimens (including pharmaceutically salts thereof) may be administered sequentially (e.g., a first regimen administered prior to administration of any doses of a second regimen). In some embodiments, the two or more chemotherapeutic regimens (including pharmaceutically salts thereof) are administered in overlapping dosing regimens.

In some embodiments, combination therapy does not necessarily require that individual agents (including pharmaceutically acceptable salts thereof) be administered together in a single composition (or even necessarily at the same time). In some embodiments, two or more therapeutic regimens (e.g., azenosertib, or a pharmaceutically acceptable salt thereof, and a second chemotherapeutic agent, or a pharmaceutically acceptable salt thereof) of a combination therapy are administered to a subject separately, e.g., in separate compositions, via separate administration routes (e.g., one agent orally and another agent intravenously), and/or at different time points. In some embodiments, two or more chemotherapeutic agents (including pharmaceutically acceptable salts thereof) may be administered together in a combination composition, or even in a combination compound (e.g., as part of a single chemical complex or covalent entity), via the same administration route, and/or at the same time.

In some embodiments, azenosertib, or a pharmaceutically acceptable salt thereof, and a second chemotherapeutic, or a pharmaceutically acceptable salt thereof, are administered concurrently. In some embodiments, azenosertib, or a pharmaceutically acceptable salt thereof, and a second chemotherapeutic, or a pharmaceutically acceptable salt thereof, are administered sequentially. In some embodiments, azenosertib, or a pharmaceutically acceptable salt thereof, is administered prior to a second chemotherapeutic, or a pharmaceutically acceptable salt thereof. In other embodiments, azenosertib, or a pharmaceutically acceptable salt thereof, is administered after a second chemotherapeutic, or a pharmaceutically acceptable salt thereof. In some embodiments, azenosertib, or a pharmaceutically acceptable salt thereof, and a second chemotherapeutic, or a pharmaceutically acceptable salt thereof, are administered intermittently.

In some embodiments, the second chemotherapeutic agent is selected from bendamustine, bortezomib, carfilzomib, ixazomib, busulfan, carboplatin, cisplatin, cyclophosphamide, cladribine, paclitaxel, docetaxel, pegylated liposomal doxorubicin (PLD), dexamethasone, doxorubicin, gemcitabine, cytarabine, fludarabine, fluorouracil (5-FU), irinotecan, topotecan, temozolomide, triapine, azacitidine, 5-azacytidine, capecitabine, AraC-FdUMP[10](CF-10), cladribine, etoposide, decitabine, daunorubicin, doxorubicin, ifosfamide, methotrexate, vincristine, hydroxyurea and oxaliplatin, or a pharmaceutically acceptable salt of any of the foregoing.

In some embodiments, the cancer treatment is alkylating agents, anti-EGFR antibodies, anti-Her-2 antibodies, antimetabolites, vinca alkaloids, platinum-based agents, anthracyclines, topoisomerase inhibitors, taxanes, antibiotics, immunomodulators, immune cell antibodies, interferons, interleukins, HSP90 inhibitors, anti-androgens, antiestrogens, anti-hypercalcaemia agents, apoptosis inducers, Aurora kinase inhibitors, Bruton's tyrosine kinase inhibitors, calcineurin inhibitors, CaM kinase II inhibitors, CD45 tyrosine phosphatase inhibitors, CDC25 phosphatase inhibitors, CHK kinase inhibitors, cyclooxygenase inhibitors, bRAF kinase inhibitors, cRAF kinase inhibitors, Ras inhibitors, cyclin dependent kinase inhibitors, cysteine protease inhibitors, DNA intercalators, DNA strand breakers, E3 ligase inhibitors, EGF Pathway Inhibitors, farnesyltransferase inhibitors, Flk-1 kinase inhibitors, glycogen synthase kinase-3 (GSK3) inhibitors, histone deacetylase (HDAC) inhibitors, I-kappa B-alpha kinase inhibitors, imidazotetrazinones, insulin tyrosine kinase inhibitors, c-Jun-N-terminal kinase (INK) inhibitors, mitogen-activated protein kinase (MAPK) inhibitors, MDM2 inhibitors, MEK inhibitors, ERK inhibitors, MMP inhibitors, mTor inhibitors, NGFR tyrosine kinase inhibitors, p38 MAP kinase inhibitors, p56 tyrosine kinase inhibitors, PDGF pathway inhibitors, phosphatidylinositol 3-kinase inhibitors, phosphatase inhibitors, protein phosphatase inhibitors, PKC inhibitors, PKC delta kinase inhibitors, polyamine synthesis inhibitors, PTP1B inhibitors, protein tyrosine kinase inhibitors, SRC family tyrosine kinase inhibitors, Syk tyrosine kinase inhibitors, Janus (JAK-2 and/or JAK-3) tyrosine kinase inhibitors, retinoids, RNA polymerase II elongation inhibitors, serine/threonine kinase inhibitors, sterol biosynthesis inhibitors, VEGF pathway inhibitors, chemotherapeutic agents, alitretinon, altretamine, aminopterin, aminolevulinic acid, amsacrine, asparaginase, atrasentan, bexarotene, carboquone, demecolcine, efaproxiral, elsamitrucin, etoglucid, hydroxycarbamide, leucovorin, lonidamine, lucanthone, masoprocol, methyl aminolevulinate, mitoguazone, mitotane, oblimersen, omacetaxine, pegaspargase, porfimer sodium, prednimustine, sitimagene ceradenovec, talaporfin, temoporfin, trabectedin and/or verteporfin, or a pharmaceutically acceptable salt of any of the foregoing.

In some embodiments, the method comprises administering a second therapeutic agent, or a pharmaceutically acceptable salt thereof.

In some embodiments, the second therapeutic agent is a PARP inhibitor, or a pharmaceutically acceptable salt thereof, wherein the PARP inhibitor is selected from the group consisting of olaparib, niraparib, rucaparib, talazoparib, veliparib, pamiparib (BGB-290), iniparib (BSI 201), saruparib (AZD5305), E7016 (Esai) and CEP-9722, or a pharmaceutically acceptable salt of any of the foregoing. In some embodiments, the second therapeutic agent is a PARP1 selective inhibitor, such as saruparib (AZD5305).

In some embodiments, azenosertib, or a pharmaceutically acceptable salt thereof, is administered in combination with a PARP inhibitor, or a pharmaceutically acceptable salt thereof. In some embodiments, the PARP inhibitor is niraparib, olaparib or saruparib (AZD5305), or a pharmaceutically acceptable salt of any of the foregoing.

In some embodiments, the PARP inhibitor is olaparib, or a pharmaceutically acceptable salt thereof. In some embodiments, the second therapeutic agent is a PD1 inhibitor, or a pharmaceutically acceptable salt thereof, wherein the PD1 inhibitor is selected from the group consisting of nivolumab, pembrolizumab, cemiplimab, spartalizumab, ABBV-181, lodapolimab, zimberelimab, toripalimab (Tuoyi), tislelizumab, camrelizumab, sintilimab (Tyvyt), GB226, AK105, HLX-10, AK103, BAT-1306, GSL-010, CS1003, LZM009 and SCT-I10A, or a pharmaceutically acceptable salt of any of the foregoing.

In some embodiments, the second therapeutic agent is a PD-L1 inhibitor, or a pharmaceutically acceptable salt thereof, wherein the PD-L1 inhibitor is selected from the group consisting of atezolizumab, avelumab, durvalumab, KN035, CS1001, SHR-1316, TQB2450, BGB-A333, KL-A167, KN046, MSB2311 and HLX-20, or a pharmaceutically acceptable salt of any of the foregoing.

In some embodiments, the second therapeutic agent is a Bcl-2 inhibitor, or a pharmaceutically acceptable salt thereof, wherein the Bcl-2 inhibitor is selected from the group consisting of ZN-d5, AGP-2575, AGP-1252, venetoclax (ABT-199), navitoclax (ABT-263), S55746/BCL201, 565487, BGB-11417, FCN-338 and AZD0466, or a pharmaceutically acceptable salt of any of the foregoing.

In some embodiments, the second therapeutic agent is a KRAS inhibitor, or a pharmaceutically acceptable salt thereof, wherein the KRAS inhibitor is selected from the group consisting of sotorasib, adagrasib, JDQ443, MRTX-1257, MRTX1133, ARS-1620, ARS-853, ARS-107, BAY-293, BI-3406, BI-2852, BMS-214662, MRTX849, MRTX849-VHL (LC2), PROTAC K-Ras Degrader-1 (Compound 518, CAS No. 2378258-52-5), Lonafarnib (SCH66336), RMC-0331, GDC-6036, LY3537982, D-1553, ARS-3248 (JNJ74699157), BI-1701963 and AU-8653 (AU-BEI-8653), or a pharmaceutically acceptable salt of any of the foregoing.

In some embodiments, the second therapeutic agent is a CDK4/6 inhibitor, or a pharmaceutically acceptable salt thereof, wherein the CDK4/6 inhibitor is selected from the group consisting of palbociclib, abemaciclib, ribociclib, trilaciclib (G1T28), lerociclib (G1T38), SHR6390, FCN-437, AMG 925, BPI-1178, BPI-16350, Birociclib, BEBT-209, TY-302, TQB-3616, HS-10342, PF-06842874, CS-3002 and MM-D37K, or a pharmaceutically acceptable salt of any of the foregoing.

In some embodiments, the second therapeutic agent is a HER-2 antibody, or a pharmaceutically acceptable salt thereof, wherein the HER-2 antibody is selected from the group consisting of trastuzumab, trastuzumab-dkst, pertuzumab and ZW25, or a pharmaceutically acceptable salt of any of the foregoing.

In some embodiments, the second therapeutic agent is a HER-2 antibody-drug conjugate, or a pharmaceutically acceptable salt thereof, wherein the HER-2 antibody-drug conjugate is selected from the group consisting of fam-trastuzumab deruxtecan-nxki, Ado-trastuzumab emtansine (T-DM1), ARX788, ALT-P7, DS8201a, MEDI4276, MM302, PF-06804103, SYD985 and XMT-1522, or a pharmaceutically acceptable salt of any of the foregoing.

In some embodiments, the second therapeutic agent is a HER2 bispecific antibody or a pharmaceutically acceptable salt thereof, wherein the HER2 bispecific antibody is selected from the group consisting of margetuximab, ertumaxomab, HER2Bi-aATC, MM-111, MCLA-128, BTRC4017A, GBR-1302 and PRS-343, or a pharmaceutically acceptable salt of any of the foregoing.

In some embodiments, the second therapeutic agent is a selective ER modulator (SERM) or a pharmaceutically acceptable salt thereof, wherein the selective ER modulator is selected from the group consisting of tamoxifen, raloxifene, ospemifene, bazedoxifene, toremifene and lasofoxifene, or a pharmaceutically acceptable salt of any of the foregoing.

In some embodiments, the second therapeutic agent is a selective ER degrader (SERD), or a pharmaceutically acceptable salt thereof, wherein the selective ER degrader is selected from the group consisting of fulvestrant, (E)-3-[3,5-Difluoro-4-[(1R,3R)-2-(2-fluoro-2-methylpropyl)-3-methyl-1,3,4,9-tetrahydropyrido[3,4-b]indol-1-yl]phenyl]prop-2-enoic acid (AZD9496), (R)-6-(2-(ethyl(4-(2-(ethylamino)ethyl)benzyl)amino)-4-methoxyphenyl)-5,6,7,8-tetrahydronaphthalen-2-ol (elacestrant, RAD1901), (E)-3-(4-((E)-2-(2-chloro-4-fluorophenyl)-1-(1H-indazol-5-yl)but-1-en-1-yl)phenyl)acrylic acid (brilanestrant, ARN-810, GDC-0810), (E)-3-(4-((2-(2-(1,1-difluoroethyl)-4-fluorophenyl)-6-hydroxybenzo[b]thiophen-3-yl)oxy)phenyl)acrylic acid (LSZ102), (E)-N,N-dimethyl-4-((2-((5-((Z)-4,4,4-trifluoro-1-(3-fluoro-1H-indazol-5-yl)-2-phenylbut-1-en-1-yl)pyridin-2-yl)oxy)ethyl)amino)but-2-enamide (H3B-6545), (E)-3-(4-((2-(4-fluoro-2,6-dimethylbenzoyl)-6-hydroxybenzo[b]thiophen-3-yl)oxy)phenyl)acrylic acid (rintodestrant, G1T48), D-0502, SHR9549, ARV-471, 3-((1R,3R)-1-(2,6-difluoro-4-((1-(3-fluoropropyl)azetidin-3-yl)amino)phenyl)-3-methyl-1,3,4,9-tetrahydro-2H-pyrido[3,4-b]indol-2-yl)-2,2-difluoropropan-1-ol (giredestrant, GDC-9545), (S)-8-(2,4-dichlorophenyl)-9-(4-((1-(3-fluoropropyl)pyrrolidin-3-yl)oxy)phenyl)-6,7-dihydro-5H-benzo[7]annulene-3-carboxylic acid (SAR439859), N-[1-(3-fluoropropyl)azetidin-3-yl]-6-[(6S,8R)-8-methyl-7-(2,2,2-trifluoroethyl)-6,7,8,9-tetrahydro-3H-pyrazolo[4,3-f]isoquinolin-6-yl]pyridin-3-amine (AZD9833), OP-1250 and LY3484356, or a pharmaceutically acceptable salt of any of the foregoing.

In some embodiments, the second therapeutic agent is an ATR inhibitor, or a pharmaceutically acceptable salt thereof, wherein the ATR inhibitor is selected from Gartisertib, Berzosertib, M4344, BAY1895344, Ceralasertib, SchisandrinB, Elimusertib, NU6027, Dactolisib, ETPPT-46464, Torin 2, VE-821, AZ20, Camonsertib, CGK733, ART-0380, ATRN-119 and ATRN-212, or a pharmaceutically acceptable salt of any of the foregoing.

In some embodiments, the second therapeutic agent is an ATM inhibitor, or a pharmaceutically acceptable salt thereof, wherein the ATM inhibitor is selected from AZD7648, AZD0156, AZ31, AZ32, AZD1390, KU55933, KU59403, KU60019, CP-466722, CGK733, NVP-BEZ235, SJ573017, AZ31, AZ32, AZD1390, M4076SKLB-197, CGK733, M4076, M3541 and M4076, or a pharmaceutically acceptable salt of any of the foregoing.

In some embodiments, the second therapeutic agent is a CHK1 inhibitor, or a pharmaceutically acceptable salt thereof, wherein the CHK1 inhibitor is selected from Prexasertib, AZD7762, Rabusertib, SCH90076MK-8776, CCT245737, CCT244747, CHIR-124, PD 407824, PD-321852, PF-00477736, GDC-0425, GDC-0575, SB-218078, V158411, LY2606368, LY2603618, SAR-020106, XL-844, UCN-01, SOL-578, IMP 10 and CBP501, or a pharmaceutically acceptable salt of any of the foregoing.

In some embodiments, the second therapeutic agent is a targeted therapeutic, or a pharmaceutically acceptable salt thereof, wherein the targeted therapeutic is selected from bevacizumab, lenvatinib, encorafenib and cetuximab, or a pharmaceutically acceptable salt of any of the foregoing.

In some embodiments, the chemotherapeutic agent is selected from carboplatin, cisplatin, paclitaxel, docetaxel, pegylated liposomal doxorubicin, doxorubicin, gemcitabine, cytarabine, fludarabine, fluorouracil (5-FU), irinotecan, topotecan, temozolomide, triapine, 5-azacytidine, capecitabine, AraC-FdUMP[10](CF-10), cladribine, decitabine, hydroxyurea and oxaliplatin, or a pharmaceutically acceptable salt of any of the foregoing. In other embodiments, the chemotherapeutic agent is azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and vincristine, or a pharmaceutically acceptable salt of any of the foregoing.

In some embodiments, the second chemotherapeutic agent is carboplatin, paclitaxel, gemcitabine or pegylated liposomal doxorubicin (PLD), or a pharmaceutically acceptable salt of any of the foregoing.

In some embodiments, the second chemotherapeutic agent is encorafenib, or a pharmaceutically acceptable salt thereof. In some embodiments, the second chemotherapeutic agent is cetuximab, or a pharmaceutically acceptable salt thereof. In some embodiments, the second chemotherapeutic agent consists of a combination of encorafenib and cetuximab, or a pharmaceutically acceptable salt of any of the foregoing.

In one aspect provided herein is a method of treating cancer comprising, administering to a subject, an effective of azenosertib, or a pharmaceutically acceptable salt thereof, at or greater than about 400 mg, or an equivalent thereof, in an intermittent dosing cycle comprising five consecutive days with dosing and two days without dosing, and administering to the subject, an effective of a PARP inhibitor (PARP inhibitor), or a pharmaceutically acceptable salt thereof, in an intermittent dosing cycle comprising at five consecutive days with dosing and two days without dosing.

### Dosing for a Second Chemotherapeutic Agent

In some embodiments, the second chemotherapeutic agent is carboplatin, paclitaxel, gemcitabine or pegylated liposomal doxorubicin (PLD), or a pharmaceutically acceptable salt of any of the foregoing.

In some embodiments, the second chemotherapeutic agent is carboplatin, or a pharmaceutically acceptable salt thereof, wherein carboplatin, or a pharmaceutically acceptable salt thereof, is administered intravenously at a dose ranging from about 1 to about 10 mg/mL*min for 15 minutes or longer once during the treatment cycle. In some embodiments, the second chemotherapeutic agent is carboplatin, or a pharmaceutically acceptable salt thereof, wherein carboplatin, or a pharmaceutically acceptable salt thereof, is administered intravenously at a dose ranging from about 3 to about 6 mg/mL*min for 15 minutes or longer once during the treatment cycle. In some embodiments, the second chemotherapeutic agent is carboplatin, or a pharmaceutically acceptable salt thereof, wherein carboplatin, or a pharmaceutically acceptable salt thereof, is administered intravenously at a dose ranging from about 1 to about 10 mg/mL*min, about 2 to about 10 mg/mL*min, about 3 to about 10 mg/mL*min, about 4 to about 10 mg/mL*min, about 5 to about 10 mg/mL*min, about 6 to about 10 mg/mL*min, about 7 to about 10 mg/mL*min, about 8 to about 10 mg/mL*min, about 9 to about 10 mg/mL*min, about 2 to about 8 mg/mL*min, about 2 to about 7 mg/mL*min, about 3 to about 7 mg/mL*min, about 4 to about 7 mg/mL*min, about 5 to about 7 mg/mL*min, about 4 to about 6 mg/mL*min, about 2 to about 6 mg/mL*min, about 3 to about 8 mg/mL*min, about 9 to about 10 mg/mL*min for 15 minutes or longer once during the treatment cycle.

In some embodiments, the second chemotherapeutic agent is PLD, or a pharmaceutically acceptable salt thereof, wherein PLD, or a pharmaceutically acceptable salt thereof, is administered intravenously at a dose ranging from about 10 to about 100 mg/m² over 60 minutes once during the treatment cycle. In some embodiments, the second chemotherapeutic agent is PLD, or a pharmaceutically acceptable salt thereof, wherein PLD, or a pharmaceutically acceptable salt thereof, is administered intravenously at a dose ranging from about 5 to about 50 mg/m² over 60 minutes once during the treatment cycle. In some embodiments, the second chemotherapeutic agent is PLD, or a pharmaceutically acceptable salt thereof, wherein PLD, or a pharmaceutically acceptable salt thereof, is administered intravenously at a dose ranging from about 10 to about 40 mg/m² over 60 minutes once during the treatment cycle.

In some embodiments, the second chemotherapeutic agent is PLD, or a pharmaceutically acceptable salt thereof, wherein PLD, or a pharmaceutically acceptable salt thereof, is administered intravenously at a dose ranging from about 10 to about 100 mg/m², about 10 to about 90 mg/m², about 10 to about 80 mg/m², about 10 to about 70 mg/m², about 10 to about 60 mg/m², about 10 to about 50 mg/m², about 10 to about 40 mg/m², about 10 to about 30 mg/m², about 10 to about 20 mg/m², about 20 to about 90 mg/m², about 30 to about 90 mg/m², about 40 to about 90 mg/m², about 50 to about 90 mg/m², about 60 to about 90 mg/m², about 70 to about 90 mg/m², about 20 to about 80 mg/m², about 20 to about 70 mg/m², about 20 to about 60 mg/m², about 20 to about 50 mg/m², about 20 to about 40 mg/m² or about 30 to about 40 mg/m² over 60 minutes once during the treatment cycle.

In some embodiments, the second chemotherapeutic agent is paclitaxel, or a pharmaceutically acceptable salt thereof, wherein paclitaxel, or a pharmaceutically acceptable salt thereof, is administered intravenously at a dose ranging from about 10 to about 120 mg/m² over 60 minutes (+10 minutes) three times during the treatment cycle. In some embodiments, the second chemotherapeutic agent is paclitaxel, or a pharmaceutically acceptable salt thereof, wherein paclitaxel, or a pharmaceutically acceptable salt thereof, is administered intravenously at a dose ranging from about 10 to about 100 mg/m², about 20 to about 100 mg/m², about 30 to about 100 mg/m², about 40 to about 100 mg/m², about 50 to about 100 mg/m², about 60 to about 100 mg/m², about 70 to about 100 mg/m², about 80 to about 100 mg/m², about 90 to about 100 mg/m², about 10 to about 90 mg/m², about 10 to about 80 mg/m², about 10 to about 70 mg/m², about 10 to about 60 mg/m², about 10 to about 50 mg/m², about 10 to about 40 mg/m², about 10 to about 30 mg/m², about 30 to about 70 mg/m², about 40 to about 70 mg/m², about 50 to about 70 mg/m², about 60 to about 70 mg/m², about 30 to about 90 mg/m² or about 30 to about 80 mg/m² administered up to 3 hours, three times during the treatment cycle.

In some embodiments, the second chemotherapeutic agent is paclitaxel, or a pharmaceutically acceptable salt thereof, wherein paclitaxel, or a pharmaceutically acceptable salt thereof, is administered intravenously at a dose ranging from about 40 to about 100 mg/m² administered up to 3 hours up to three times during treatment cycles.

In some embodiments, the second chemotherapeutic agent is gemcitabine, or a pharmaceutically acceptable salt thereof, wherein gemcitabine, or a pharmaceutically acceptable salt thereof, is administered intravenously at a dose ranging from about 500 to about 1500 mg/m² over 15 minutes longer once during the treatment cycle.

In some embodiments, the second chemotherapeutic agent is gemcitabine, or a pharmaceutically acceptable salt thereof, wherein gemcitabine, or a pharmaceutically acceptable salt thereof, is administered intravenously at a dose ranging from about 100 to about 1000 mg/m², about 100 to about 1000 mg/m², about 100 to about 900 mg/m², about 100 to about 800 mg/m², about 100 to about 700 mg/m², about 100 to about 600 mg/m², about 100 to about 500 mg/m², about 100 to about 400 mg/m², about 100 to about 300 mg/m², about 100 to about 200 mg/m², about 200 to about 1000 mg/m², about 300 to about 1000 mg/m², about 400 to about 1000 mg/m², about 500 to about 1000 mg/m², about 600 to about 1000 mg/m², about 700 to about 1000 mg/m², about 800 to about 1000 mg/m², about 200 to about 800 mg/m², about 200 to about 700 mg/m², about 200 to about 600 mg/m², about 200 to about 500 mg/m², about 300 to about 900 mg/m², about 300 to about 800 mg/m², about 400 to about 700 mg/m², about 500 to about 700 mg/m², about 500 to about 800 mg/m² or about 600 to about 900 mg/m² over 15 min or longer up to 3 times during the treatment cycle.

In some embodiments, the second chemotherapeutic agent is gemcitabine, or a pharmaceutically acceptable salt thereof, wherein gemcitabine, or a pharmaceutically acceptable salt thereof, is administered intravenously at a dose ranging from about 100 to about 1000 mg/m² over 15 min or longer up to 3 times during the treatment cycle.

### Responsiveness

In some embodiments, the treatment methods described herein results in a response rate at or greater than 10%, 15%, 20%, 25%, 30%, 35%, 40%, 45% or 50%. In some embodiments, the response rate is measured by complete response (CR), partial response (PR), CA-125 50% response, or combination thereof. In some embodiments, response is determined based on progression free survival. In some embodiments, response is determined based on tumor response. In some embodiments, response is determined based on clinical benefit rate (CBR). In some embodiments, response is determined based on disease control rate (DCR). In some embodiments, response is determined based on overall survival (OS).

Progression free survival (PFS) refers to the time period for which a subject having a disease (e.g., cancer) survives, without a significant worsening of the disease state. Progression free survival may be assessed as a period of time in which there is no progression of tumor growth and/or wherein the disease status of a subject is not determined to be a progressive disease. In embodiments, progression free survival of a subject having cancer is assessed by evaluating tumor size, tumor number and/or metastasis.

In some embodiments, the treatment results in progression-free survival (PFS) of 5 months, 6 months, 7 months, 8 months, 9 months, 10 months, 11 months, 12 months or longer. In some embodiments, the treatment results in progression-free survival (PFS) of 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months or longer. In some embodiments, the treatment results in progression-free survival (PFS) of 1 year, 1.5 years, 2 years, 2.5 years, or longer.

As used herein, the term “progression” of tumor growth or a “progressive disease” (PD) as used herein in reference to cancer status indicates an increase in the sum of the diameters of the target tumors. Progression for the purposes of determining progression free survival may also be determined if at least one of the following criteria is met: 1) tumor assessment by CT/MRI unequivocally shows progressive disease according to RECIST 1.1 criteria; or 2) additional diagnostic tests (e.g. histology/cytology, ultrasound techniques, endoscopy, positron emission tomography) identify new tumors or determine existing tumors qualify for unequivocal progressive disease and/or CA-125-progression according to Gynecologic Cancer Intergroup (GCIG)-criteria (see Rustin et al., Int J Gynecol Cancer 2011; 21: 419-423 which is incorporated herein in its entirety); 3) definitive clinical signs and symptoms of PD unrelated to non-malignant or iatrogenic causes ([i]intractable cancer-related pain; [ii] malignant bowel obstruction/worsening dysfunction; or [iii] unequivocal symptomatic worsening of ascites or pleural effusion) and/or CA-125-progression according to GCIG-criteria.

As used herein, the term “partial response” or “PR” refers to a decrease in tumor progression in a subject as indicated by a decrease in the sum of the diameters of the target tumors, taking as reference the baseline sum diameters. In embodiments, PR refers to at least a 30% decrease in the sum of diameters, taking as reference the baseline sum diameters. Exemplary methods for evaluating partial response are identified by RECIST guidelines. See E. A. Eisenhauer, et al., “New response evaluation criteria in solid tumors: Revised RECIST guideline (version 1.1),” Eur. J. of Cancer, 45: 228-247 (2009).

As used herein, “stabilization” of tumor growth or a “stable disease” (SD) refers to neither sufficient shrinkage to qualify for PR nor sufficient increase to qualify for PD. In embodiments, stabilization refers to a less than 30%, 25%, 20%, 15%, 10% or 5% change (increase or decrease) in the sum of the diameters of the target tumors, taking as reference the baseline sum diameters. Exemplary methods for evaluating stabilization of tumor growth or a stable disease are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

As used herein, the term “complete response” or “CR” is used to mean the disappearance of all or substantially all target lesions. In embodiments, CR refers to an 80%, 85%, 90%, 91%, 92%, 93%, 94%, 95%, 96%, 97%, 98%, 99% or 100% decrease in the sum of the diameters of the target tumors (i.e., loss of tumors), taking as reference the baseline sum diameters. In embodiments, CR indicates that less than 10%, 9%, 8%, 7%, 6%, 5%, 4%, 3%, 2%, 1% or less of the total lesion diameter remains after treatment. Exemplary methods for evaluating complete response are identified by RECIST guidelines. See E. A. Eisenhauer, et al., “New response evaluation criteria in solid tumors: Revised RECIST guideline (version 1.1.),” Eur. J. of Cancer, 45: 228-247 (2009).

### Examples

Additional embodiments are disclosed in further detail in the following examples, which are not in any way intended to limit the scope of the claims.

### Example 1—HRD-Positive Status is Associated with Tumor Response in Azenosertib-Treated Cancer Subjects

This example demonstrates the effects of azenosertib on HRD+cancers (e.g., cancers with one or more HRRm) in tumor response and progression free survival. Subjects with uterine serous carcinoma (USC) or high-grade serous ovarian cancer (HGSOC) were treated with azenosertib and grouped by azenosertib dose of lower than 300 mg (LT300) or at or greater than 300 mg azenosertib (300+mg). Table 1 shows the subject selection based on HRRm status. Total subjects in parenthesis includes all subjects in the study including subjects receiving LT300.

TABLE 1
Subject selection based on HRRm status

| Study | Total | HRRm | HRRm wt |
|---|---|---|---|
| 001 - USC | 19(19) | 4 | 15 |
| 004 - USC | 35(26) | 8 | 27 |
| 001 - HGSOC | 20(18) | 6 | 14 |
| All | 74(63) | 18 | 56 |

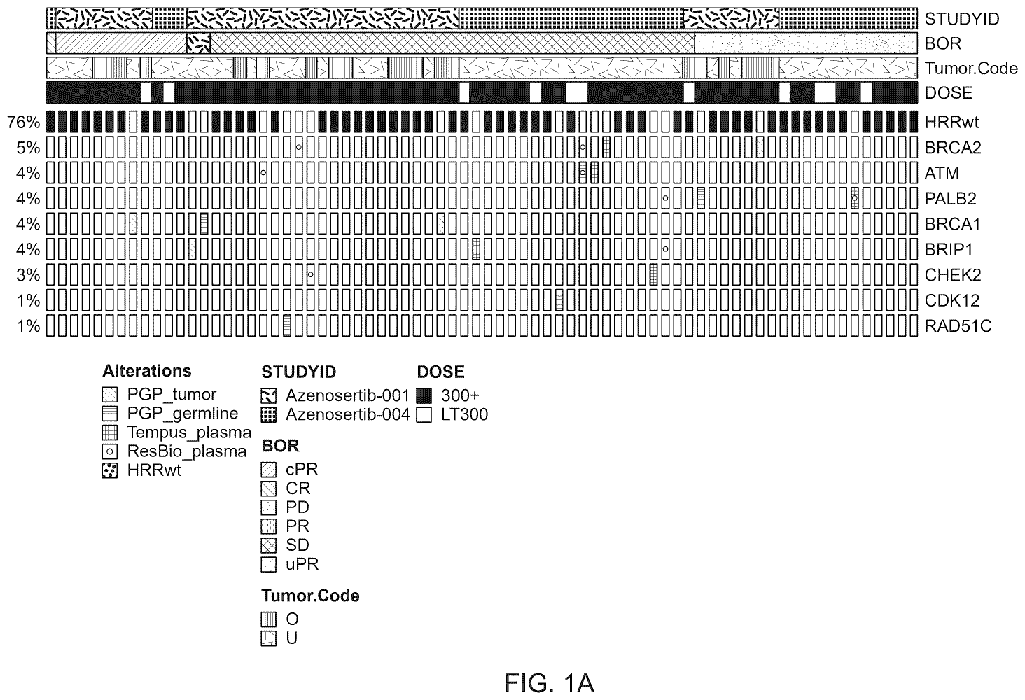

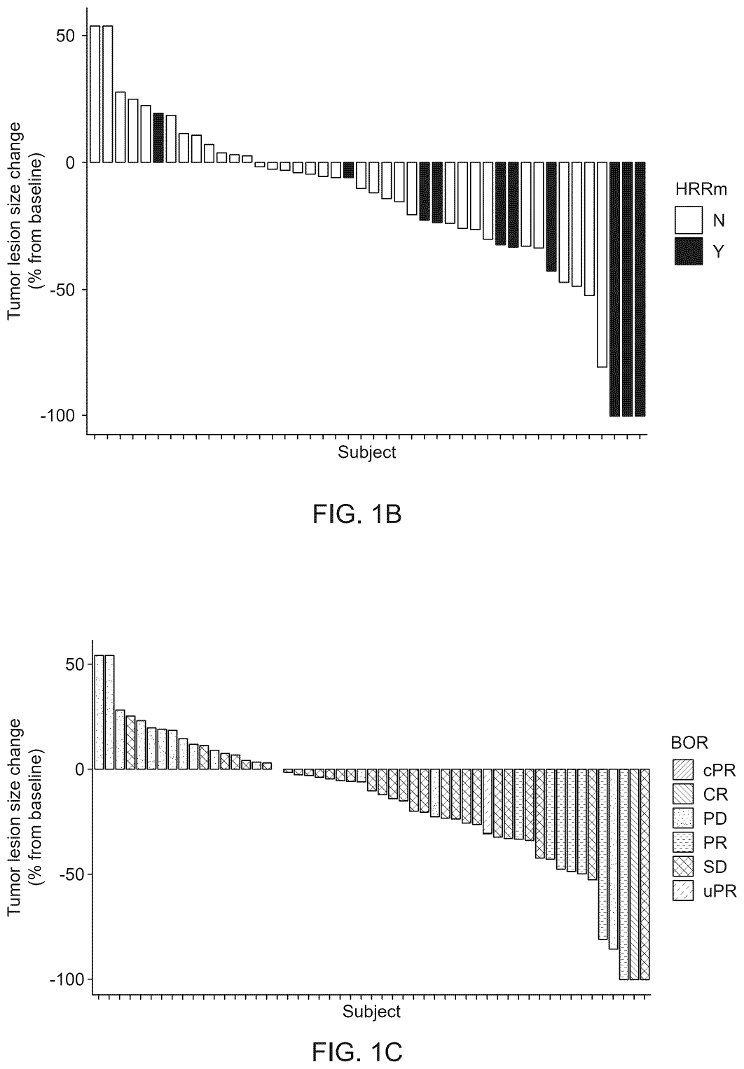

Subjects selected in Table 1 were evaluated for tumor response when treated with doses of 300 mg or more of azenosertib or with lower than 300 mg of azenosertib. In the USC population treated with azenosertib of 300 mg or more (300+), HRRm positivity was associated with better tumor response (p=0.01). (FIG. 1B and FIG. 1C). The HRRm median was −33% and HRR wt median was −5%. FIG. 1A shows the genotype and dosage of individual subjects. Subjects treated with 300+mg azenosertib, demonstrated that HRRm status as a biomarker was associated with better progression free survival. HRRm subject population was associated with 8.3 months of progression free survival, compared to HRR wt subject population with 4.2 months progression free survival. (FIG. 1D). HRRm status was associated with better progression free survival in subjects treated with at least 300 mg azenosertib, supporting an effective dose of 300 mg or more in HRD subjects.

### Example 2—In Vivo Efficacy of Azenosertib in Prostate Cancer 22RV1 Subcutaneous Xenografted Mice

This example demonstrates in vivo therapeutic efficacy of azenosertib alone or in combination with a PARP inhibitor (olaparib or niraparib) in 22RV1 (prostate cancer cell line) subcutaneous xenografts in CB17 SCID mice.

The prostate cancer cell line 22RV1 harbors a BRCA2 pathogenic mutation (HRD+). Table 2 summarizes the groups and treatments. Briefly, 72 tumor-bearing mice were randomized and assigned into nine groups using a randomized block design based on their tumor volumes with eight mice per group. Each mouse was inoculated subcutaneously at the right flank with 22RV1 tumor cells in 0.2 mL mixture of Matrigel (5×10⁶ cells/mouse). Treatments were started when the tumor size reached 199.94 mm³ for tumor efficacy studies (Day 14 post inoculation).

TABLE 2
Treatment groups and dosage regimens

| | | | Dose | | |
|---|---|---|---|---|---|
| | | | Vol or | | |
| | | Dose | Quantity | | |
| Groups | Treatment | (mg/kg) | (mL/kg) | Route | Frequency |
| 1 | Vehicle | — | 10 | p.o. | qd × 5 3 cycle |
| 2 | Azenosertib | 60 | 10 | p.o. | qd × 5 3 cycle |
| 3 | Azenosertib | 80 | 10 | p.o. | qd × 5 3 cycle |
| 4 | Niraparib | 30 | 10 | p.o. | qd × 5 3 cycle |
| 5 | Azenosertib + | 60 mg/kg | 10 + 10 | p.o. + | qd × 5 3 cycle + |
| | Niraparib | azenosertib + | | p.o. | qd × 5 3 cycle |
| | | 30 mg/kg | | | |
| | | niraparib | | | |
| 6 | Azenosertib + | 80 mg/kg | 10 + 10 | p.o. + | qd × 5 3 cycle + |
| | niraparib | azenosertib + | | p.o. | qd × 5 3 cycle |
| | | 30 mg/kg | | | |
| | | niraparib | | | |
| 7 | Olaparib | 50 | 10 | p.o. | qd × 5 3 cycle |
| 8 | Azenosertib + | 60 mg/kg | 10 + 10 | p.o. + | qd × 5 3 cycle + |
| | olaparib | azenosertib + | | p.o. | qd × 5 3 cycle |
| | | 50 mg/kg | | | |
| | | olaparib | | | |
| 9 | Azenosertib + | 80 mg/kg + | 10 + 10 | p.o. + | qd × 5 3 cycle + |
| | olaparib | 50 mg/kg | | p.o. | qd × 5 3 cycle |
| | | olaparib | | | |

1. QD dosing: Mice grouping was on day 1 and treatment was started from the afternoon of day 1 (D 1). Qd × 5 = 5 days on; 2 days off.

Tumor size was measured twice weekly in two dimensions using caliper and expressed in mm3 using the formula:

$$V = \frac{a \times {b}^{2}}{2} ,$$

where a and b were long and short diameters of the tumor. The tumor size was used for calculations of T/C %. The T/C(%) was calculated according to the following formula:

$$\frac{T}{C ( \% )} = \frac{{T}_{RTV}}{{C}_{RTV}} \times 100 \% ,$$

$${T}_{RTV} = \frac{{TV}_{treatment - Dn}}{{TV}_{treatment - D 1}} ,$$

$${C}_{RTV} = \frac{{TV}_{control - Dn}}{{TV}_{control - D 1}} ,$$

$$TGIv ( \% ) = ( 1 - \frac{( 1 - {TV}_{\frac{treatment}{Dx}} - {TV}_{\frac{treatment}{D 1}} )}{( {TV}_{\frac{Control}{Dx}} - {TV}_{\frac{Control}{D 1}} )} \times 100 \%$$

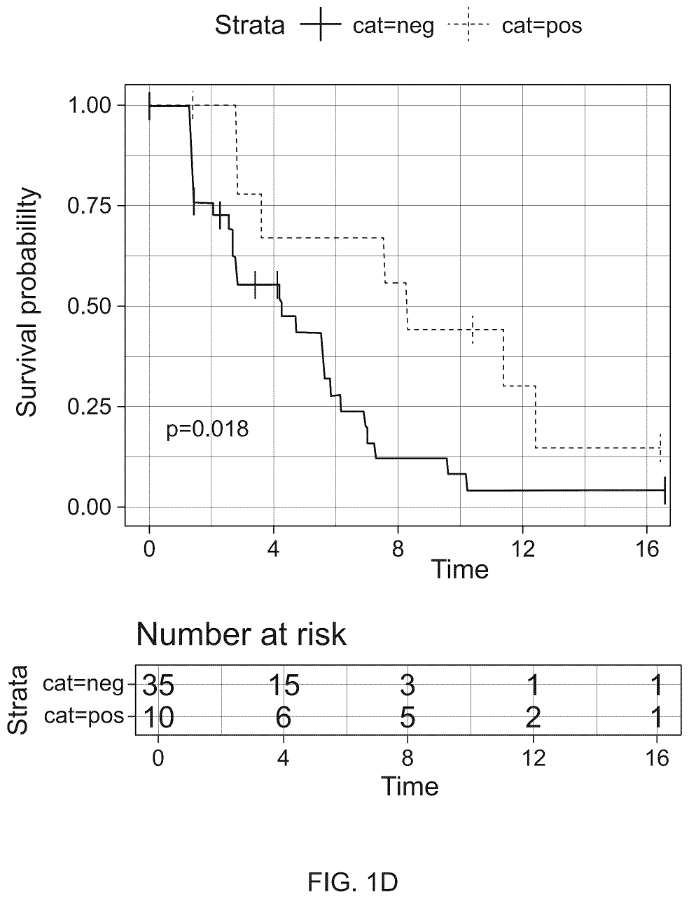

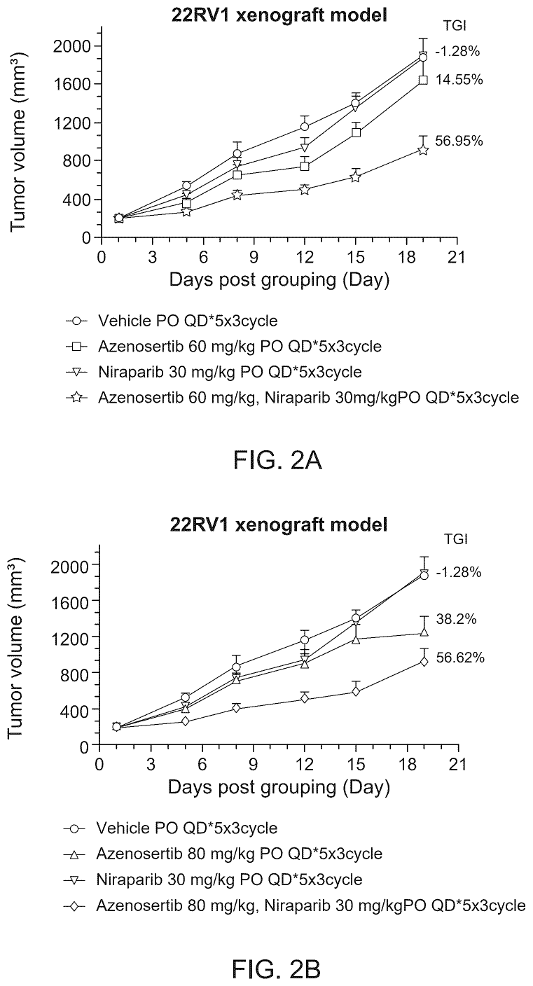

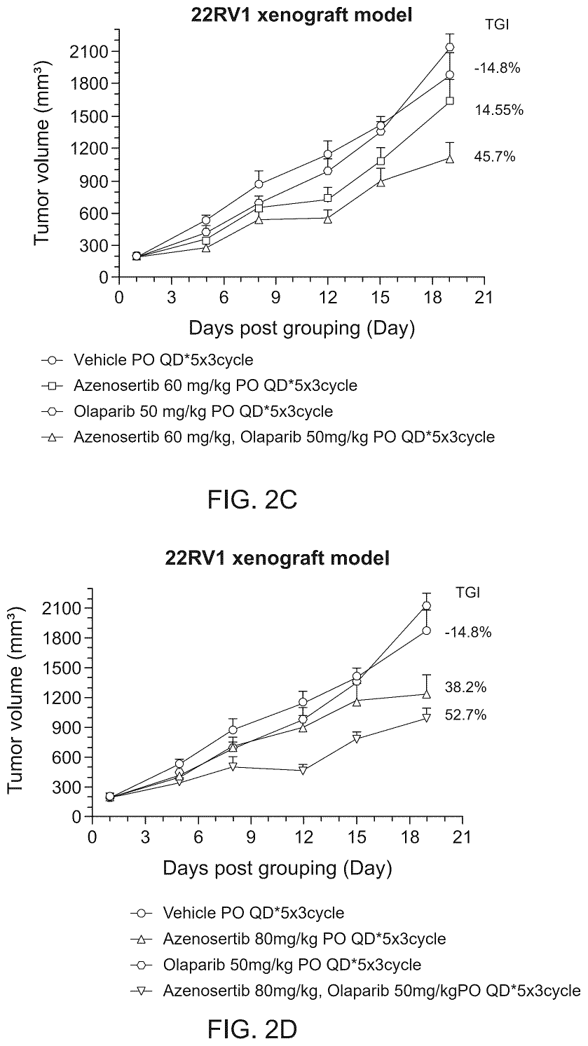

Two-way ANOVA followed by Bonferroni post-test was performed to compare tumor volumes among vehicle and other groups. FIGS. 2A-2D and Table 3 shows the tumor growth inhibition (% TGI) during the treatment period.

TABLE 3
Tumor growth inhibition (% TGI)

| | | % TGI | | | | |
|---|---|---|---|---|---|---|
| | | Day | Day | Day | Day | Day |
| Group | Treatment | 5 | 8 | 12 | 15 | 19 |
| 1 | Vehicle | — | — | — | — | — |
| 2 | Azenosertib | 54.08% | 32.68% | 44.02% | 26.61% | 14.55% |
| | (60 mg/kg) | | | | | |
| 3 | Azenosertib | 38.26% | 23.80% | 26.16% | 19.47% | 38.13% |
| | (80 mg/kg) | | | | | |
| 4 | Niraparib | 31.64% | 18.17% | 21.99% | 4.28% | −1.28% |
| | (30 mg/kg) | | | | | |
| 5 | Azenosertib | 78.53% | 64.87% | 68.49% | 63.62% | 56.95% |
| | (60 mg/kg) + | | | | | |
| | niraparib | | | | | |
| | (30 mg/kg) | | | | | |
| 6 | Azenosertib | 81.5% | 69.13% | 67.9% | 67.76% | 56.62% |
| | (80 mg/kg) + | | | | | |
| | niraparib | | | | | |
| | (30 mg/kg) | | | | | |
| 7 | Olaparib | 33.81% | 26.80% | 18.2% | 5.03% | 14.79% |
| | 50 mg/kg | | | | | |
| 8 | Azenosertib | 75.34% | 48.47% | 62.15% | 42.22% | 45.73% |
| | (60 mg/kg) + | | | | | |
| | olaparib | | | | | |
| | (50 mg/kg) | | | | | |
| 9 | Azenosertib | 56.27% | 53.17% | 71.47% | 51.28% | 52.69 W % |
| | (80 mg/kg) + | | | | | |
| | olaparib | | | | | |
| | (50 mg/kg) | | | | | |

Overall, results showed that azenosertib, alone and in combination with niraparib or olaparib resulted in reduction of tumor growth volume and tumor inhibition.

### Example 3—In Vivo Anti-Tumor Efficacy of Azenosertib in Patient-Derived Xenograft (PDA) Breast Cancer Models

This example demonstrates anti-tumor efficacy of azenosertib and niraparib, used alone or in combination, in the HBCx-9, HBCx-10 and HBCx-17 triple-negative breast cancer (TNBC) patient-derived xenograft (PDX) models, using immunodeficient female mice.

HRD+TNBC PDX models (HBCx-10 and HBCx-17) and an HRD-TNBC PDX model (HBCx-9), whose HRD status and profiles are shown in Table 4, were treated with azenosertib and/or niraparib. Animals were treated with 60 mg/kg azenosertib or 35 mg/kg niraparib alone or in combination on an intermittent dosing regimen of once a day for 5 days followed by 2 days off (qd x5, 2 days off) for 4 cycles (28 days).

TABLE 4
Characteristics and HRD status of patient-derived TNBC models

| TNBC model | Ki67 | Her2 | HRD status | Key mutations |
|---|---|---|---|---|
| HBCx-9 | 86% | 0 | Likely − | TP53, ATM (vus), CDH1 |
| HBCx-10 | 94% | 1+ | + | TP53, BRCA2, PTEN |
| | | | | del, RB1 del |
| HBCx-17 | 94% | 0 | + | TP53, AKT1, BRCA2, |
| | | | | CDKN2A, KDM6A del |

Table 5 summarizes the groups and dosing schedule. Treatments were initiated 28 days post implantation of the tumor cells. The study period ended 28 days after the start of treatment.

TABLE 5
Treatment groups and dose regimens

| | | | Dose | | |
|---|---|---|---|---|---|
| | | Dose | volume | | |
| Group | Treatment | (mg/kg) | (mL/kg) | Route | Schedule |
| 1 | Vehicle | — | 10 | p.o. | (5 days on, 2 days |
| | | | | | off) × 4* |
| 2 | Azenosertib | 60 mg/kg | 10 | p.o. | (5 days on, 2 days |
| | | | | | off) × 4* |
| 3 | Niraparib | 35 mg/kg | 10 | p.o. | (5 days on, 2 days |
| | | | | | off) × 4* |
| 4 | Azenosertib + | 60 mg/kg | 10 + 10 | p.o. + | (5 days on, 2 days |
| | niraparib | azenosertib + | | p.o. | off) × 4* |
| | | 35 mg/kg | | | |
| | | niraparib | | | |

*Daily administered by per os route at 10 mL/kg for 5 consecutive days a week. 4 treatments cycles were performed (e.g., D 0 to D 4, D 7 to D 11, D 14 to D 18 then D 21 to D 25; 20 doses in total).

Relative body weight (RBW) is calculated for each measurement by dividing the body weight by the body weight at the start of treatment. Individual body weight loss percent is given by the formula:

$$( \% BWL ) = 100 - \frac{{BW}_{x}}{{BW}_{0}} \times 100 ,$$

where BWx is the BW at any day during the treatment and BW0 is the BW on the day of inclusion.

Mean body weight loss percent is given by the formula:

$$( \% BWL ) = 100 - \frac{mean {BW}_{x}}{mean {BW}_{0}} \times 100 ,$$

where BWx is the mean BW at any day during the treatment and BW0 is the mean BW on the day of inclusion.

Mean relative body weight (RBW) curves were obtained by plotting the mean RBW against time for each experimental group. Delta relative body weights (relative body weights of treated group compared to relative body weights of control group) were used for statistical analysis. Mean individual body weights are shown in Table 6.

TABLE 6
Mean and individual relative body weight

| | Max. | | Max. | |
|---|---|---|---|---|
| | Mean | At | Individual | At |
| Group | BWL | Day | BWL | day |
| 1 | 0.9% | 3 | 5.0% | 10 |
| 2 | 2.6% | 3 | 9.5% | 24 |
| 3 | 3.4% | 3 | 6.2% | 3 |
| 4 | 6.0% | 10 | 12.5% | 10 |

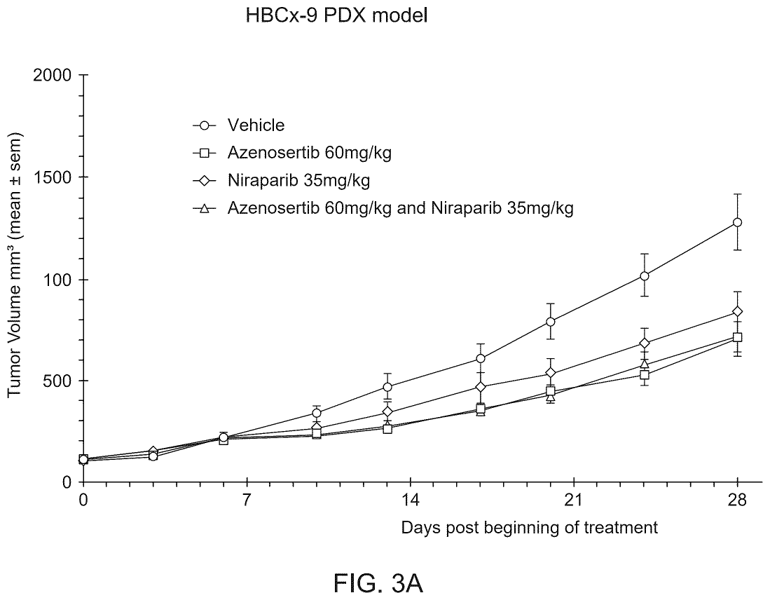

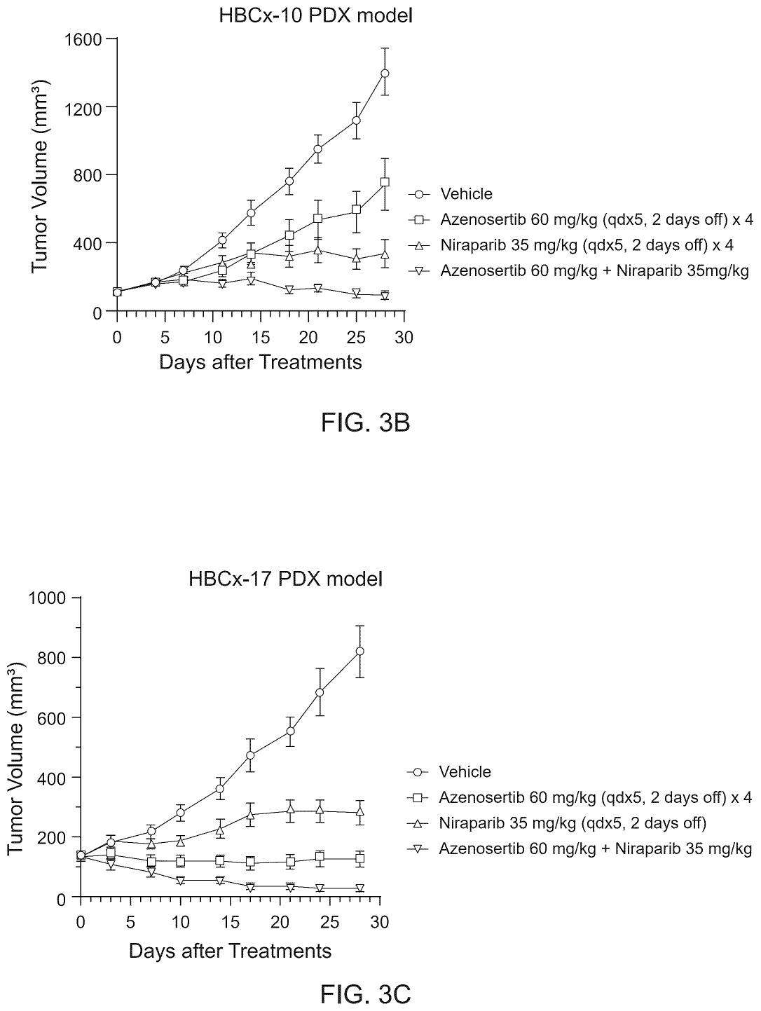

Combination treatment resulted in statistically significant reduction in tumor volume. Body weight data and clinical observations of azenosertib at 60 mg/kg and niraparib at 35 mg/kg were well tolerated as single agents or in combination and did not induce any relevant body weight loss or observable clinical signs. Azenosertib and the combination of azenosertib and niraparib provided increased tumor growth inhibition in HRD+models (FIG. 3B and FIG. 3C) compared to the HRD-model (FIG. 3A).

### Example 4—In Vivo Efficacy of Azenosertib and PARP Inhibitor Combination in BRCA Mutant Ovarian PDX Models

This example demonstrates anti-tumor efficacy of azenosertib and niraparib, used alone or in combination, in patient-derived ovarian xenograft (PDX) models harboring BRCA1 and/or BRCA2 mutations. The OVA2-BUR model has an HRD negative/BRCA1 WT phenotype. Tumor growth inhibition was also measured using BRCA1^{Glu1607Ter} (pathogenic) mutant and BRCA1^{Pro1099Leu} (Benign) mutant PDX models. Table 7 shows the treatment groups, dosage and schedule.

TABLE 7
Treatment groups and dosage schedule

| | | Dose | Dose volume | | |
|---|---|---|---|---|---|
| Group | Treatment | (mg/kg) | (mL/kg) | Route | Frequency |
| 1 | Vehicle | — | 10 | p.o. | (5 days on, 2 days |
| | | | | | off) × 4* |
| 2 | Azenosertib | 60 | 10 | p.o. | (5 days on, 2 days |
| | | | | | off) × 4* |
| 3 | Niraparib | 35 | 10 | p.o. | (5 days on, 2 days |
| | | | | | off) × 4* |
| 4 | Azenosertib + | 60 mg/kg | 10 + 10 | p.o. + | (5 days on, 2 days |
| | niraparib | azenosertib + | | p.o. | off) × 4* |
| | | 35 mg/kg | | | |
| | | niraparib | | | |

*daily administered by per os route at 10 mL/kg for 5 consecutive days a week. 4 treatments cycles were performed (e.g., D 0 to D 4, D 7 to D 11, D 14 to D 18 then D 21 to D 25; 20 doses in total).

Relative Body Weight (RBW), individual body weight and mean body weight loss percent were calculated for each measurement by dividing the body weight by the body weight at the start of treatment as described in Example 3. Mean individual body weight are shown in Table 8.

TABLE 8
Mean and individual relative body weight

| | Max. | | Max. | |
|---|---|---|---|---|
| | mean | At | individual | At |
| Group | BWL | Day | BWL | day |
| 1 | 0.7% | 4 | 4.1% | 11 |
| 2 | 0.7% | 11 | 7.6% | 15 |
| 3 | 1.5% | 4 | 7.0% | 4 |
| 4 | 8.2% | 11 | 15.3% | 28 |

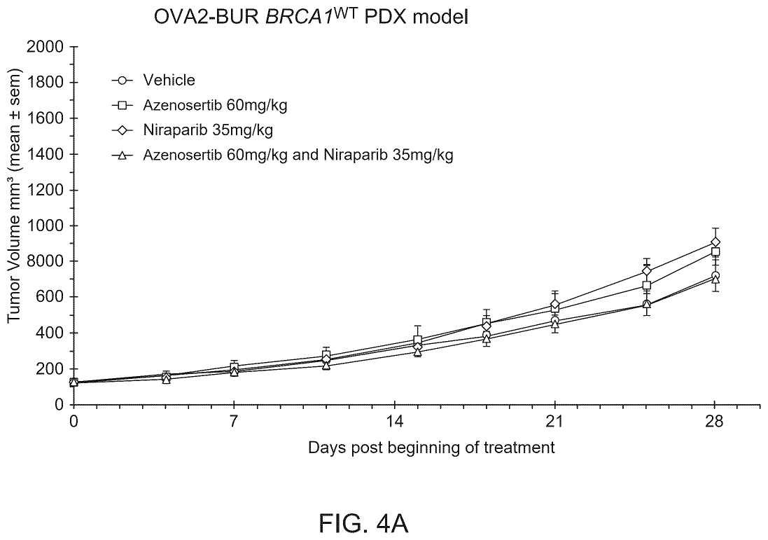

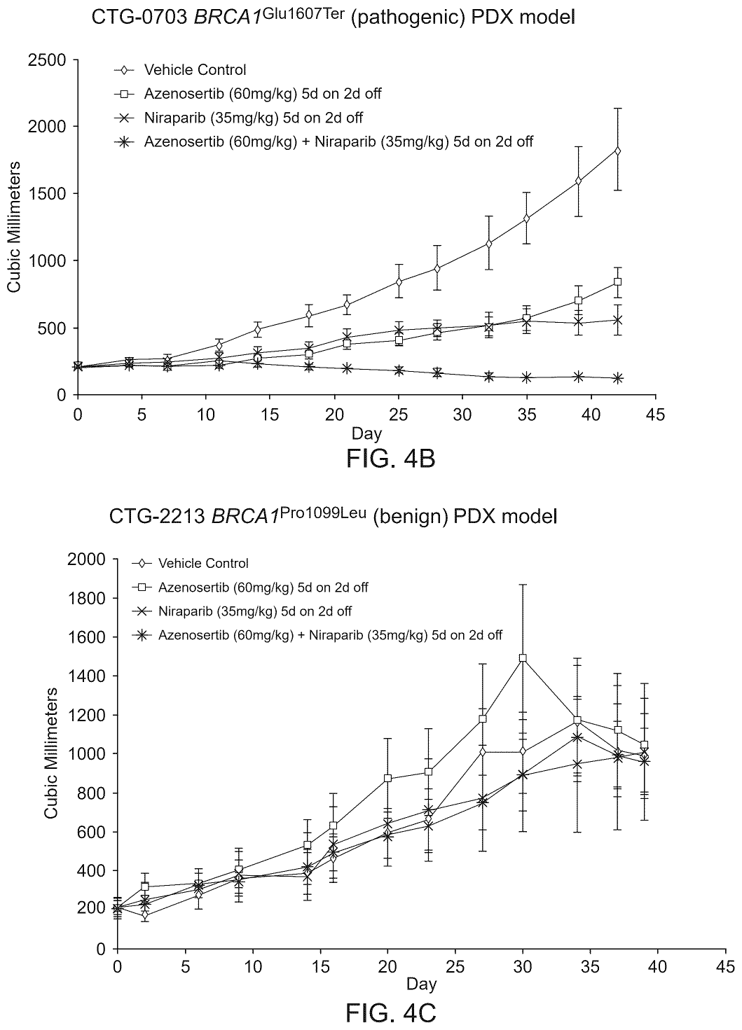

FIG. 4A shows the tumor volume change using azenosertib, niraparib alone or in combination. Body weight data and clinical observations of azenosertib at 60 mg/kg and niraparib at 35 mg/kg demonstrate that these compounds were well tolerated as single agents or in combination and did not induce any relevant body weight loss or observable clinical signs. Combination treatment also resulted in statistically significant reduction in tumor volume in BRCA1 mutant (HRD+) models. Interestingly, azenosertib alone or in combination with niraparib provided increased tumor growth inhibition in an HRD+(e.g., BRCA1 pathogenic mutant shown in FIG. 4B) ovarian PDX model compared to the BRCA1 WT (FIG. 4A) or benign BRCA1 mutant (FIG. 4C) models.

### Example 5—Efficacy of Azenosertib in HRD+Breast Cancer with Acquired PARP Inhibitor-Resistance

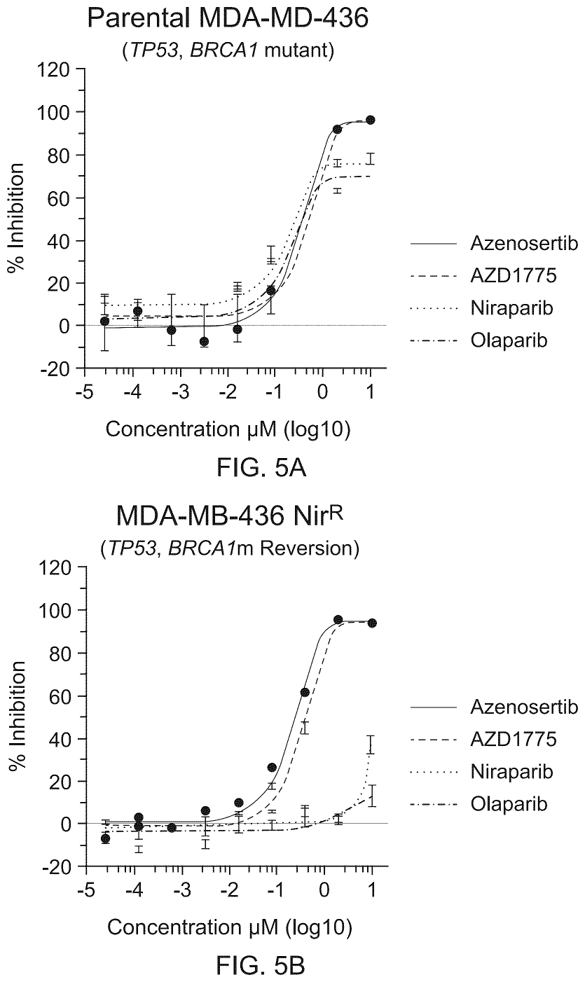

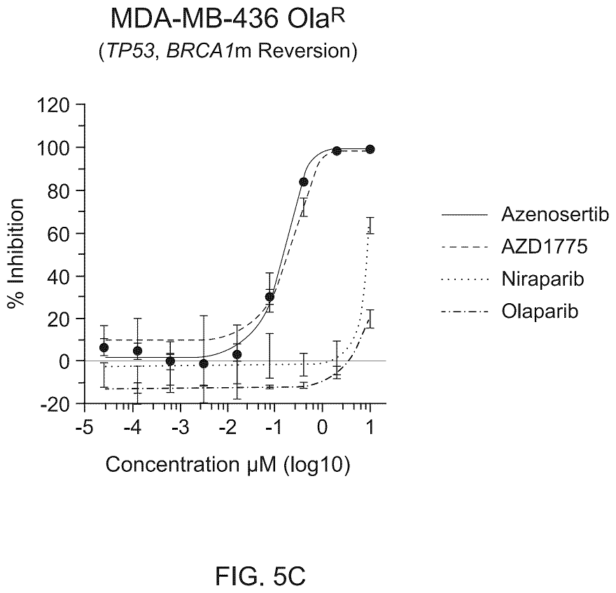

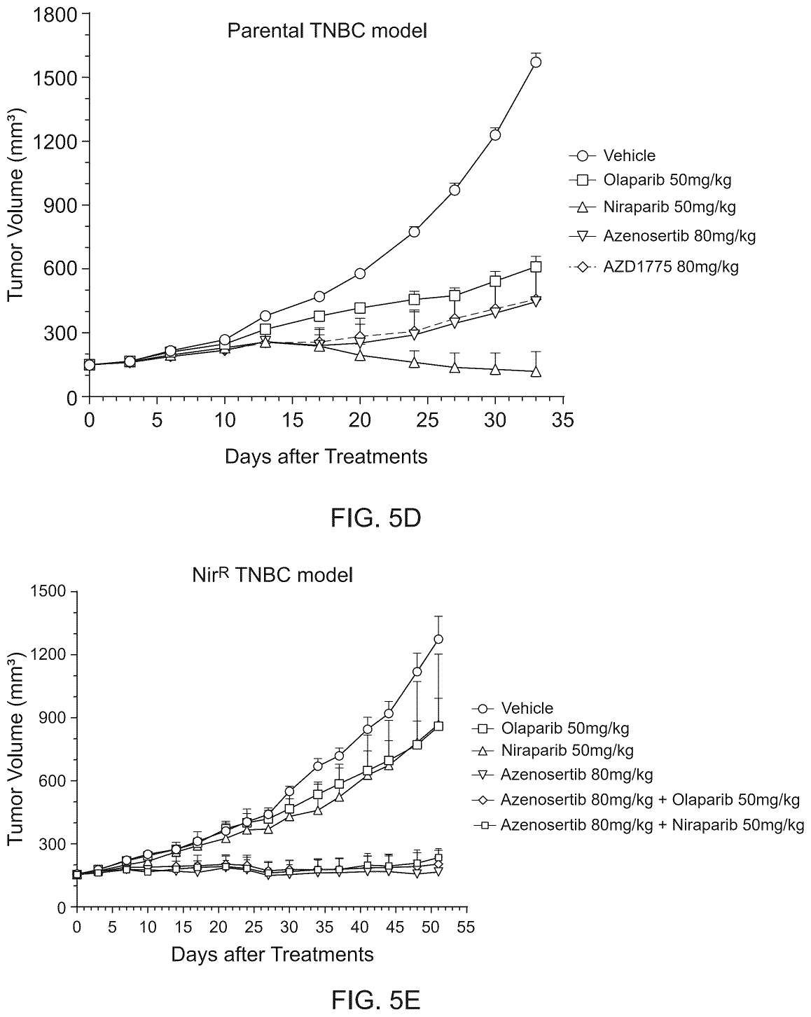

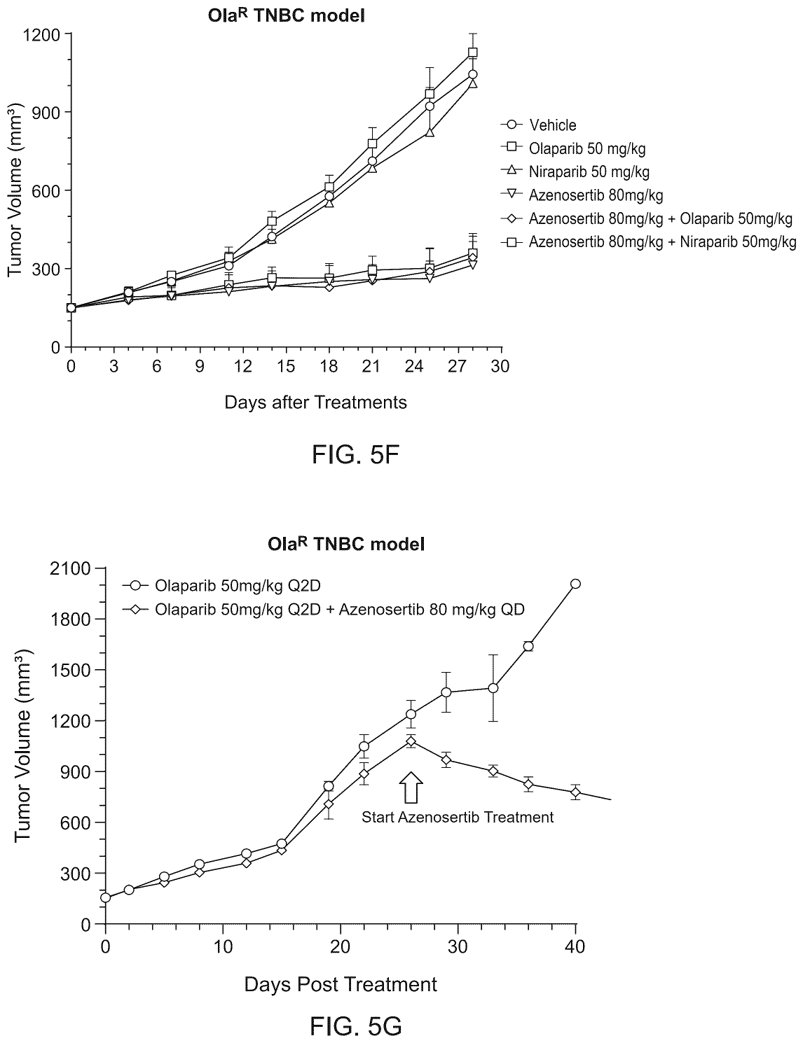

This example demonstrates efficacy of azenosertib in HRD+-xenograft cancer models with acquired resistance to PARP inhibitors. Azenosertib is active in PARP inhibitor-resistant xenograft models with a BRCA reversion mutation. Cells with BRCA reversion mutations may be considered HRD+due to genome instability (such as TAI, LST, LOH). BRCA reversion mutation is an established mechanism of resistance to both platinum-based chemotherapeutic drugs and PARP inhibitors. In vivo exploration of WEE1 inhibitors (azenosertib, adavosertib) and PARP inhibitors (niraparib, olaparib) was evaluated. The anti-tumor activity of azenosertib or PARP inhibitors, was evaluated in the parental MDA-MB-436 triple negative breast cancer tumor cell line (FIG. 5A) or in two derivative lines of MDA-MB-436 with in vitro acquired resistance to niraparib (Nir^{R}, FIG. 5B) or olaparib (Ola^{R}, FIG. 5C) and as shown in Table 9.

TABLE 9
In vitro activity (IC₅₀) of azenosertib in parental and
PARP inhibitor-resistant MDA-MB-436 TNBC cell lines.

| | | Olaparib^{R} | Niraparib^{R} |
|---|---|---|---|
| | Wild | (BRCA | (BRCA |
| | Type | reversion) | reversion) |
| Azenosertib | 282 | 174 | 185 |
| Niraparib | 103 | 7731 | 8648 |
| Olaparib | 95 | >10000 | >10000 |
| AZD6738 (ATRi) | 3668 | 1842 | 1770 |
| AZD0156 (ATMi) | 4979 | 1986 | 2076 |
| AZD7648 (DNA-PKi) | >10000 | >10000 | >10000 |
| MK8776 (CHK1i) | >10000 | 5969 | ND |

MDA-MB-436 PARP inhibitor-resistant lines were generated by long-term in vitro treatment with a PARP inhibitor that is olaparib (Ola^{R}) or niraparib (Nir^{R}). Resistant cells were confirmed by sequencing to have BRCA1 reversion mutation. The parental MDA-MB-436 cell line remains sensitive to PARP inhibitors as expected (FIG. 5A), while the Nir^{R} (FIG. 5B) and Ola^{R} (FIG. 5C) MDA-MB-436 cell lines are no longer sensitive to PARP inhibitors but are sensitive to azenosertib. Azenosertib activity was further confirmed in the PARP inhibitor-resistant MDA-MB-436 CDX model in vivo. Azenosertib administered at 80 mg/kg in the MDA-MB-436 niraparib-resistant model (FIG. 5E) and the MDA-MB-436 olaparib-resistant model (FIG. 5F and FIG. 5G) achieved significant reduction in tumor volume compared to the PARP inhibitor. WEE1 inhibitor or PARP inhibitor treatment in the Parental TNBC animal model (without PARP inhibitor resistance) is shown in FIG. 5D. Taken together, these data demonstrate that olaparib- and niraparib-resistant models remain sensitive to WEE1 inhibition but not PARP inhibitor.

Moreover, these data suggest that azenosertib may overcome PARP inhibitor resistance in subjects with BRCA reversion mutation.

### Example 6—Efficacy of Azenosertib in Human Subject with HRD+Platinum-Resistant Ovarian Cancer and Also with PARP Inhibitor-Resistance

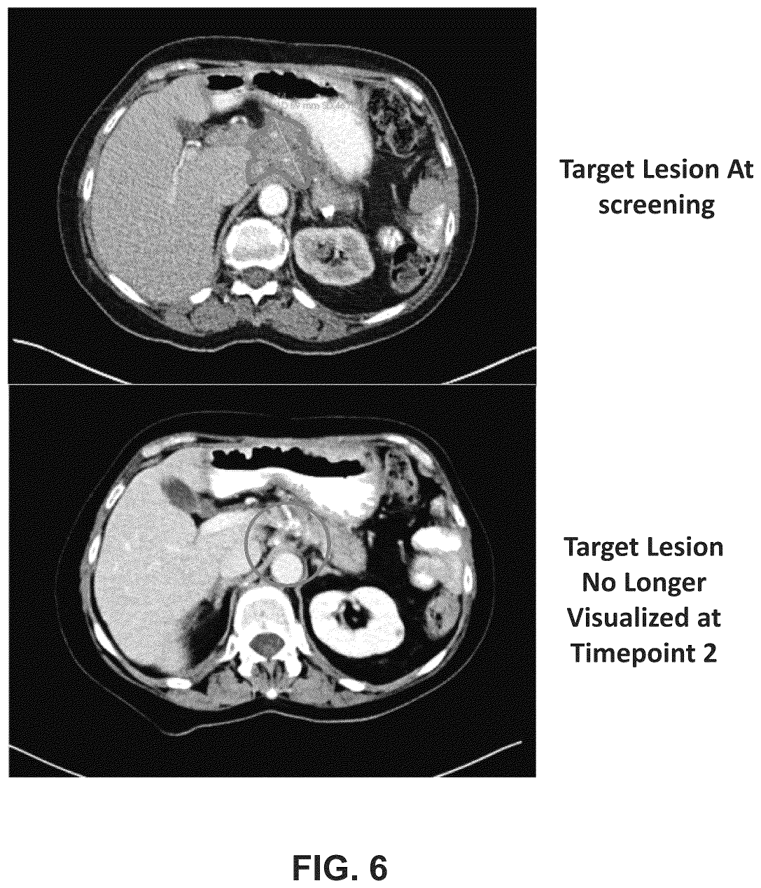

A subject with HGSOC selected to have HRD+ status (e.g., BRCA1m) confirmed by qualitative next-generation sequencing based in vitro diagnostic test (FoundationOne®CDx) was administered azenosertib in accordance with an intermittent dosing schedule of at least 400 mg azenosertib once a day for five consecutive days, followed by two off days. The subject received azenosertib for five months. The subject is a 64-year-old female who received 7 prior lines of therapy: (1) carboplatin/paclitaxel/bevacizumab/olaparib (PD); (2) Pembrolizumab (PD); (3) NaPi2b targeting-ADC (XMT-1536) (PD); (4) carboplatin/gemcitabine/bevacizumab (PD); (5) Pegylated doxorubicin (PD); (6) Topotecan (PD); (7) PABP-1 RNP (ATRC-101) (PD). After treatment with azenosertib, the subject demonstrated cPR of −48%. As shown in FIG. 6, the subject's target lesion was no longer visualized.

These data demonstrate that azenosertib alone is effective in both HRD+ PARP inhibitor-sensitive and PARP inhibitor-resistant cancers. Additionally, azenosertib in combination with a PARP inhibitor can restore anti-tumor effect in PARP inhibitor-resistant HRD+ cancers.

### Example 7—HRRm Status Associated with Better Overall Survival in Human Subjects with Uterine Serous Carcinoma (USC)

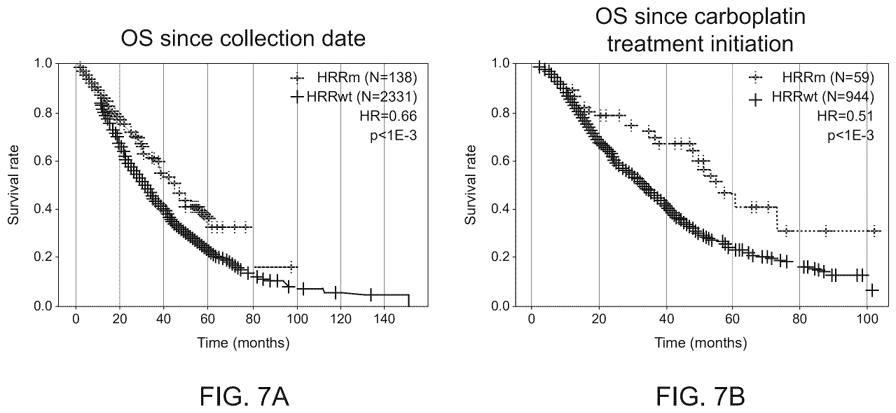

Overall survival (OS) analysis of HRRm and HRRwt or HRRm-negative subjects with USC (selected for TP53-mutated status) from the Caris Life Science database (serous endometrial carcinoma), (FIGS. 7A and 7B) was performed. For FIG. 7A, OS was measured since sample collection. For FIG. 7B, OS was measured since the initiation of Carboplatin treatment. The OS analysis indicates that HRRm subjects had better overall survival.

### Example 8—In Vitro Efficacy of Azenosertib in Additional PARP Inhibitor-Sensitive and PARP Inhibitor-Resistant Cell Line Models

Azenosertib, olaparib and AZD5305 (PARP1-selective inhibitor) were individually tested in vitro in a panel of HRD+ cells as indicated in Table 10. Briefly, cells were seeded in their recommended media at a density of 1,000 cells per well into 96 well plates. After 24 hr, drug treatment was performed and cells were incubated in a 37° C. for 5 cell doublings. Cell viability was measured using CellTiter-Glo (CTG) (Promega) according to the manufacturer's recommendations. Percent viability was calculated as percentage of cell viability relative to DMSO-only vehicle control. Dose-response curves were fit with Graphpad Prism 9 to determine IC₅₀ values.

TABLE 10
HRD+ CDX models

| | | | | PARP Inhibitor |
|---|---|---|---|---|
| Cell line | Indication | Subtype | Mutations | Sensitivity |
| UWB1289 | Ovarian | Serous Ovarian | TP53, BRCA1 | Sensitive |
| | | Cancer | | |
| HCC1937 | Breast | TNBC | TP53, BRCA1, FAM35A | Resistant |
| COV362 | Ovarian | High-Grade | TP53, BRCA1 | Resistant |
| | | Serous Ovarian | | |
| | | Cancer | | |
| HCC1569 | Breast | ER-negative | TP53, BRCA2 | Resistant |
| | | HER2-positive | | |
| HCC1395 | Breast | TNBC | TP53, BRCA1, BRCA2 | Sensitive |

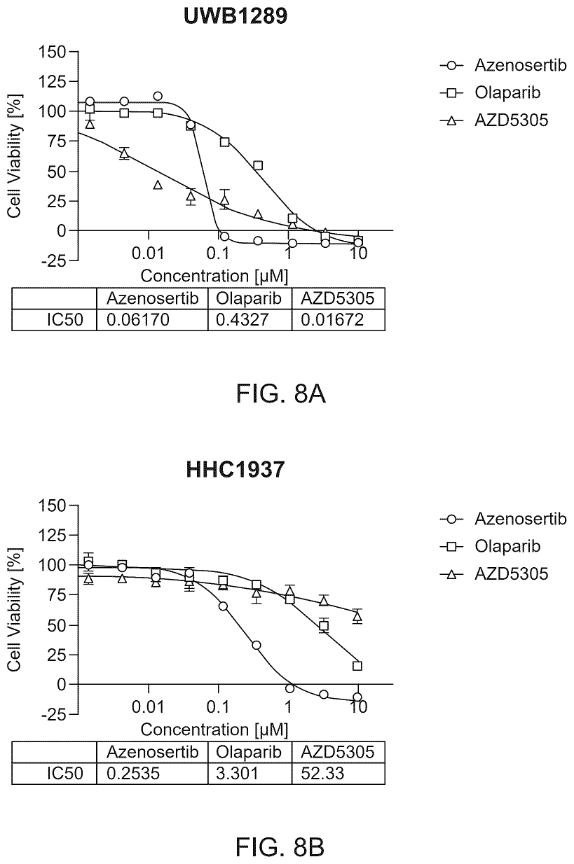

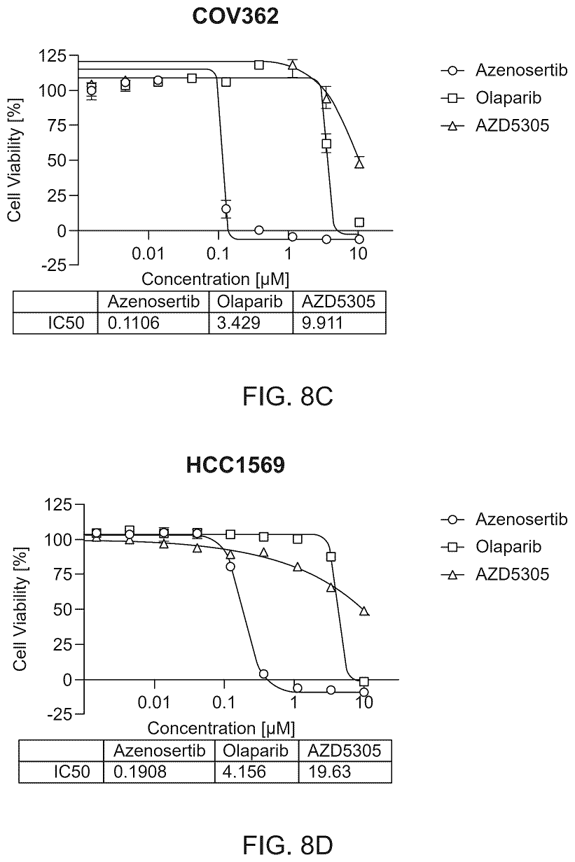

As shown in the dose response curves of FIGS. 8A-8E, azenosertib reduces tumor cell growth in HRD+ cells that are both PARP inhibitor-sensitive and PARP inhibitor-resistant. The resistance to PARP inhibitors of the cell lines HCC1937, COV362 and HCC1569 are confirmed by the olaparib and AZD5305 curves and IC₅₀ values in FIGS. 8B-8D. The PARP inhibitor resistance of HCC1937 is conferred by the FAM35A inactivation mutation.

### Example 9—In Vivo Anti-Tumor Efficacy of Azenosertib Monotherapy and Azenosertib+PARP Inhibitor Combination Therapy in PARP Inhibitor-Resistant Triple Negative Breast Cancer Cell Line-Derived Xenograft (CDX) Models

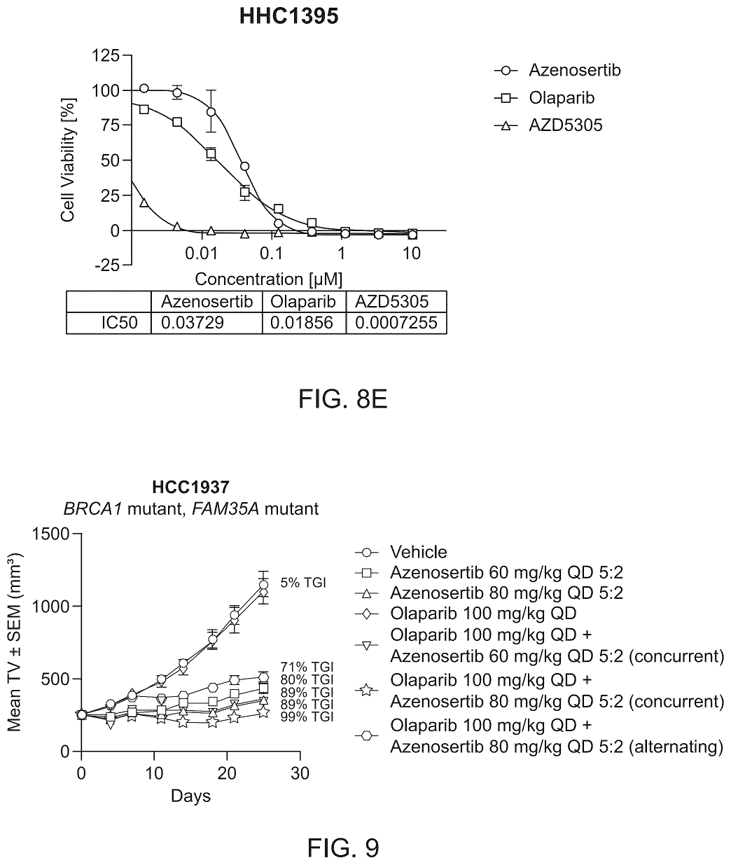

The studies in Example 9 demonstrate the anti-tumor activity of azenosertib in PARP inhibitor-resistant xenograft models of HRD+TNBC. Anti-tumor activity of azenosertib monotherapy or in combination with olaparib was evaluated in the HCC1937 model in vivo in mice (FIG. 9), and Table 11 summarizes the groups and treatments. This example demonstrates efficacy of azenosertib in a BRCA-mutant TNBC model that is resistant to PARP inhibitor due to a mutation in FAM35A as described above. Treatment with monotherapy olaparib at 100 mg/kg QD confirmed HCC1937 resistance to the PARP inhibitor. Monotherapy azenosertib at 60 or 80 mg/kg QD 5:2 achieved significant tumor growth inhibition of 80% and 89%, respectively. Concurrent combination treatment with olaparib at 100 mg/kg QD and azenosertib at either 60 or 80 mg/kg QD 5:2 also achieved significant tumor growth inhibition of 89% or 99%, respectively. Furthermore, combination treatment with alternating/sequential weeks of olaparib at 100 mg/kg QD and azenosertib at 80 mg/kg QD 5:2 was tested, but demonstrated less efficacy than both concurrent combination and continuous monotherapy with azenosertib. These data suggest that azenosertib can overcome PARP inhibitor resistance in BRCA-mutant TNBC, including BRCA-mutant TNBC with a mutation in FAM35A.

TABLE 11
HCC1937-xenografted mice treatment groups and dosage regimens

| | | Dose | Dose Vol | | |
|---|---|---|---|---|---|
| Groups | Treatment | (mg/kg) | (mL/kg) | Route | Frequency |
| 1 | Vehicle | — | 10 | p.o. | qd |
| 2 | Azenosertib | 60 | 10 | p.o. | qd × 5, 2 d off |
| 3 | Azenosertib | 80 | 10 | p.o. | qd × 5, 2 d off |
| 4 | Olaparib | 100 | 10 | p.o. | qd |
| 5 | Olaparib + | 100 mg/kg | 10 + 10 | p.o. + | qd + qd × 5, |
| | azenosertib | Olaparib + | | p.o. | 2 d off |
| | (concurrent) | 60 mg/kg | | | (concurrent) |
| | | Azenosertib | | | |
| 6 | Olaparib + | 100 mg/kg | 10 + 10 | p.o. + | qd + qd × 5, |
| | azenosertib | Olaparib + | | p.o. | 2 d off |
| | (concurrent) | 80 mg/kg | | | (concurrent) |
| | | azenosertib | | | |
| 7 | Olaparib + | 100 mg/kg | 10 + 10 | p.o. + | qd (1^{st} and 3^{rd} weeks) + |
| | azenosertib | Olaparib + | | p.o. | qd × 5, 2 d off |
| | (alternating) | 80 mg/kg | | | (2^{nd} and 4^{th} weeks) |
| | | azenosertib | | | (alternating) |

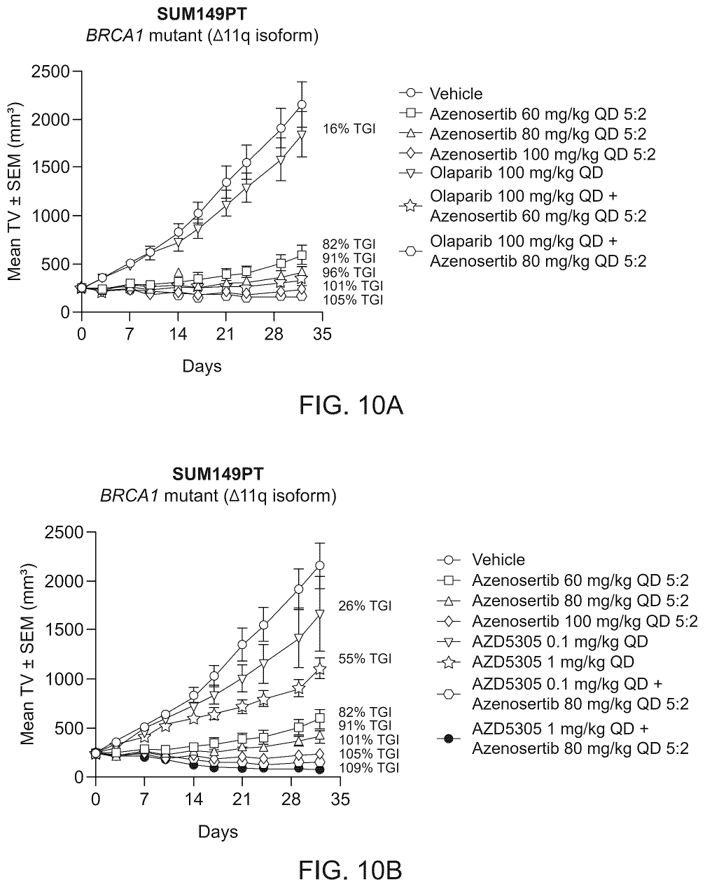

SUM149PT is a TNBC xenograft model harboring a BRCA1 splice isoform (Δ11q) that produces a truncated, partially functional protein which thereby promotes PARP inhibitor resistance. Azenosertib monotherapy and its combination with olaparib or AZD5305 was evaluated in the SUM149PT model in vivo in mice (FIGS. 10A-10B), and Table 12 summarizes the groups and treatments. Single agent treatment with olaparib at 100 mg/kg QD (FIG. 10A) or with the PARP1 selective inhibitor, AZD5305, at 0.1 or 1 mg/kg QD (FIG. 10B) confirmed SUM149PT resistance to PARP inhibitors. Monotherapy azenosertib at 60, 80 and 100 mg/kg QD 5:2 achieved 82%, 91% and 101% TGI, respectively. Concurrent combination of olaparib at 100 mg/kg QD with azenosertib at 60 or 80 mg/kg QD 5:2 achieved 96% and 105% TGI, respectively (FIG. 10A), whereas concurrent combination of AZD5305 at 0.1 or 1 mg/kg QD with azenosertib at 80 mg/kg QD 5:2 achieved 105% and 109% TGI, respectively (FIG. 10B). These data suggest that azenosertib can overcome PARP inhibitor resistance in BRCA-mutant TNBC, including BRCA-mutant TNBC with a mutation in FAM35A, including BRCA-mutant TNBC with a BRCA1 splice isoform (Δ11q).

TABLE 12
SUM149PT-xenografted mice treatment
groups and dosage regimens

| | | Dose | Dose Vol | | |
|---|---|---|---|---|---|
| Groups | Treatment | (mg/kg) | (mL/kg) | Route | Frequency |
| 1 | Vehicle | — | 10 | p.o. | qd |
| 2 | Azenosertib | 60 | 10 | p.o. | qd × 5, 2 d off |
| 3 | Azenosertib | 80 | 10 | p.o. | qd × 5, 2 d off |
| 4 | Azenosertib | 100 | 10 | p.o. | qd × 5, 2 d off |
| 4 | Olaparib | 100 | 10 | p.o. | qd |
| 5 | AZD5305 | 0.1 | 10 | p.o. | qd |
| 6 | AZD5305 | 1 | 10 | p.o. | qd |
| 5 | Olaparib + | 100 mg/kg | 10 + 10 | p.o. + | qd + qd × 5, |
| | azenosertib | Olaparib + | | p.o. | 2 d off |
| | (concurrent) | 60 mg/kg | | | (concurrent) |
| | | Azenosertib | | | |

Those skilled in the art will recognize or be able to ascertain using no more than routine experimentation, many equivalents to the specific embodiments of the invention described herein. The scope of the present disclosure is not intended to be limited to the above Description, but rather is as set forth in the following claims.

1. A method of treating cancer comprising:
administering an effective dose of azenosertib, or a pharmaceutically acceptable salt thereof, to a subject selected to have a cancer with a homologous recombination repair deficiency (HRD).

2. The method of claim 1, wherein the HRD is due to a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L.

3. A method of treating cancer comprising:
administering an effective dose of azenosertib, or a pharmaceutically acceptable salt thereof, to a subject selected to have a cancer with a homologous recombination repair deficiency-positive (HRD-positive or HRD+) status.

4. The method of claim 3, wherein the HRD-positive (HRD+) status is due to a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L.

5. A method of treating a cancer comprising:
determining or having determined whether a subject has a homologous repair deficiency (HRD) or a HRD-positive (HRD+) status; and
if the subject has the HRD or the HRD-positive (HRD+) status, administering an effective dose of azenosertib, or a pharmaceutically acceptable salt thereof, to the subject;
wherein the administration of azenosertib, or a pharmaceutically acceptable salt thereof, results in the inhibition of the cancer in the subject.

6. The method of claim 5, wherein the HRD or the HRD-positive (HRD+) status is due to a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L.

7. The method of any one of claims 1 to 6, wherein the effective dose of azenosertib, or a pharmaceutically acceptable salt thereof, is about 200 to about 450 mg/day, or about 200 to about 400 mg/day, or between about 200 to about 350 mg/day, or between about 250 to about 400 mg/day, or between about 250 to about 350 mg/day, or an equivalent of any of the foregoing.

8. The method of any one of claims 1 to 7, wherein the effective dose of azenosertib, or a pharmaceutically acceptable salt thereof, is at least about 250 mg/day, or an equivalent thereof.

9. The method of any one of claims 1 to 8, wherein the effective dose of azenosertib, or a pharmaceutically acceptable salt thereof, is at least about 250 to about 300 mg/day, or an equivalent thereof.

10. The method of any one of claims 1 to 8, wherein the effective dose of azenosertib, or a pharmaceutically acceptable salt thereof, is at least about 300 to about 350 mg/day, or an equivalent thereof.

11. The method of any one of claims 1 to 8, wherein the effective dose of azenosertib, or a pharmaceutically acceptable salt thereof, is at least about 350 to about 400 mg/day, or an equivalent thereof.

12. The method of any one of claims 1 to 11, wherein the effective dose of azenosertib, or a pharmaceutically acceptable salt thereof, is administered once a day (QD).

13. The method of any one of claims 1 to 12, wherein the effective dose of azenosertib, or a pharmaceutically acceptable salt, thereof is administered on an intermittent dosing schedule.

14. The method of claim 13, wherein the intermittent dosing schedule comprises 5 days with dosing and 2 days without dosing in each of one or more dosing weeks.

15. The method of claim 13 or 14, wherein the intermittent dosing schedule comprises 5 consecutive days with dosing and 2 consecutive days without dosing in each of one or more dosing weeks.

16. The method of any one of claims 1 to 15, wherein the HRD or HRD-positive (HRD+ status) is due to the cancer having a homologous recombination repair mutation (HRRm).

17. The method of claim 16, wherein the HRRm is a mutation in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L.

18. The method of claim 17, wherein the HRD or HRD-positive (HRD+ status) is due to the cancer having a mutation in BRCA1 and/or BRCA2.

19. The method of claim 18, wherein the HRD or HRD-positive (HRD+ status) is due to the cancer having a BRCA1 pathogenic mutation.

20. The method of claim 19, wherein the BRCA1 pathogenic mutation is BRCA1^{Glu1607Ter}.

21. The method of any one of claims 1 to 20, wherein the HRD or HRD-positive (HRD+ status) is due to the cancer having has a homologous recombination repair reversion mutation.

22. The method of claim 21, wherein the homologous recombination repair reversion mutation is in a gene selected from the group consisting of BRCA1, BRCA2, ATM, BARD1, BRIP1, CDK12, CHEK1, CHEK2, FANCL, PALB2, RAD51B, RAD51C, RAD51D and RAD54L.

23. The method of any one of claims 1 to 22, wherein the cancer has an additional mutation in a gene selected from the group consisting of TP53, AKT1, BRCA2, CDKN2A, KDM6A, PTEN, RB1 and FAM35A.

24. The method of any one of claims 1 to 23, wherein the HRD or HRD-positive (HRD+ status) is due to the cancer having a BRCA1 splicing isoform.

25. The method of claim 24, wherein the BRCA1 splicing isoform is a BRCA1-Δ11q splicing isoform.

26. The method of any one of claims 1 to 25, wherein the method comprises administering an effective dose of azenosertib, or a pharmaceutically acceptable salt thereof, to a subject selected to have a cancer with a BRCA1/2-mutant or BRCA1/2-positive status.

27. The method of any one of claims 1 to 26, wherein the method comprises administering to the subject an effective dose of azenosertib, or a pharmaceutically acceptable salt thereof, in combination with an effective dose of a PARP inhibitor, or a pharmaceutically acceptable salt thereof.

28. The method of claim 27, wherein the PARP inhibitor is niraparib, or a pharmaceutically acceptable salt thereof.

29. The method of claim 27, wherein the PARP inhibitor is olaparib, or a pharmaceutically acceptable salt thereof.

30. The method of claim 27, wherein the PARP inhibitor is a PARP1 selective inhibitor, or a pharmaceutically acceptable salt thereof.

31. The method of claim 30, wherein the PARP1 selective inhibitor is saruparib (AZD5305), or a pharmaceutically acceptable salt thereof.

32. The method of any one of claims 1 to 31, wherein the subject has received one or more prior lines of therapy.

33. The method of any one of claims 1 to 32, wherein the cancer is platinum-resistant.

34. The method of any one of claims 1 to 33, wherein the cancer is PARP inhibitor-resistant.

35. The method of claim 33 or 34, wherein the subject has previously received a PARP inhibitor, or a pharmaceutically acceptable salt thereof.

36. The method of any one of claims 1 to 35, wherein the cancer selected from the group consisting of glioblastoma, (GBM) astrocytoma, meningioma, craniopharyngioma, medulloblastoma, other brain cancers, head and neck cancer, leukemia, AML (Acute Myeloid Leukemia), CLL (Chronic lymphocytic leukemia), ALL (Acute Lymphocytic Leukemia), myelodysplastic syndromes (MDS), skin cancer, adrenal cancer, anal cancer, bile duct cancer, bladder cancer, bone cancer, breast cancer, cervical cancer, colon cancer, colorectal cancer, endometrial cancer, endometrium cancer, esophagus cancer, eye cancer, gallbladder cancer, gastric cancer, gastrointestinal cancer, Hodgkin lymphoma, Non-Hodgkin lymphoma, hematological tumor, head cancer, heme malignancy, Kaposi sarcoma, kidney cancer, laryngeal and hypopharyngeal cancer, liver cancer, lung cancer, non-small cell lung cancer (NSCLC), small cell lung cancer (SCLC), lymphoma, mesothelioma, melanoma, multiple myeloma, neuroblastoma, nasopharyngeal cancer, neck cancer, ovarian cancer, osteosarcoma, sarcomas, gastrointestinal stromal tumor (GIST), pancreatic cancer, pituitary cancer, prostate cancer, renal cancer, retinoblastoma, salivary gland cancer, skin cancer, stomach cancer, small intestine cancer, spleen cancer, sarcomas, testicular cancer, thymus cancer, thyroid cancer, uterine cancer, uterine sarcoma, uterine serous carcinoma (USC), uterine CS, vaginal cancer, vulvar cancer, Waldenstrom macroglobulinemia, Wilms tumor, solid tumor, or liquid tumor, HGSOC, invasive breast cancer, Triple Negative Breast Cancer (TNBC), esophagogastric cancer, gastric cancer, esophageal cancer, pRCC, ccRCC, chromophobe RCC, head and neck cancer, adenoid cystic carcinoma (ACC), Diffuse large B cell lymphoma (DLBCL), non-Hodgkin lymphoma (NHL), Low-grade gliomas (LGGs), Pheochromocytoma and paraganglioma (PCPGs), cholangiocarcinoma, acute myeloid leukemia (AML), CLL (Chronic lymphocytic leukemia), ALL (Acute Lymphocytic Leukemia), myelodysplastic syndromes (MDS), thymoma, BRAF mutant metastatic colorectal cancer and uveal melanoma.

37. The method of claim 36, wherein the cancer is ovarian cancer.

38. The method of claim 37, wherein the epithelial ovarian cancer is high grade serous ovarian cancer (HGSOC).

39. The method of claim 36, wherein the cancer is breast cancer.

40. The method of claim 39, wherein the breast cancer is triple negative breast cancer.

41. The method of claim 39, wherein the breast cancer is a HER2-expressing or a HER2-positive (HER2+) breast cancer.

42. The method of claim 36, wherein the cancer is endometrial cancer.

43. The method of claim 36, wherein the cancer is uterine serous carcinoma (USC).

44. The method of any one of claims 1 to 35, wherein the cancer is peritoneal cancer (e.g., primary peritoneal cancer).

45. The method of any one of claims 1 to 35, wherein the cancer is fallopian tube cancer.

46. A method of treating PARP inhibitor-resistant breast cancer comprising:
determining or having determined whether a subject has a homologous repair deficiency (HRD) or a HRD-positive (HRD+) status; and
if the subject has the HRD or the HRD-positive (HRD+) status, administering an effective dose of azenosertib, or a pharmaceutically acceptable salt thereof, to the subject;
wherein the administration of azenosertib, or a pharmaceutically acceptable salt thereof, results in the inhibition of the cancer in the subject.

47. The method of claim 46, wherein the HRD or HRD-positive (HRD+ status) is due to the PARP inhibitor-resistant breast cancer having a BRCA1 reversion mutation.

48. The method of claim 46, wherein the HRD or HRD-positive (HRD+ status) is due to the PARP inhibitor-resistant breast cancer having a BRCA1 mutation.

49. The method of any one of claims 46 to 48, wherein the HRD or HRD-positive (HRD+ status) is due to the cancer having a BRCA1 splicing isoform.

50. The method of claim 49, wherein the BRCA1 splicing isoform is a BRCA1-Δ11q splicing isoform.

51. The method of any one of claims 47 to 50, wherein the cancer has an additional mutation in the gene FAM35A.

52. The method of any one of claims 47 to 51, wherein the cancer has an additional mutation in the gene TP53.

53. The method of any one of claims 46 to 52, wherein the effective dose of azenosertib, or a pharmaceutically acceptable salt thereof, is at least about 250 mg/day, or an equivalent thereof.

54. The method of any one of claims 46 to 53, wherein the effective dose of azenosertib, or a pharmaceutically acceptable salt thereof, is at least about 250 to about 300 mg/day, or an equivalent thereof.

55. The method of any one of claims 46 to 53, wherein the effective dose of azenosertib, or a pharmaceutically acceptable salt thereof, is at least about 300 to about 350 mg/day, or an equivalent thereof.

56. The method of any one of claims 46 to 55, wherein the method comprises administering to the subject an effective dose of azenosertib, or a pharmaceutically acceptable salt thereof, in combination with an effective dose of a PARP inhibitor, or a pharmaceutically acceptable salt thereof.

57. The method of claim 56, wherein the PARP inhibitor is niraparib, olaparib or saruparib, or a pharmaceutically acceptable salt of any of the foregoing.

58. The method of any one of claims 46 to 57, wherein the PARP inhibitor-resistant breast is niraparib-resistant.

59. The method of any one of claims 46 to 57, wherein the PARP inhibitor-resistant breast is olaparib-resistant.

60. The method of any one of claims 46 to 59, wherein the effective dose of azenosertib, or a pharmaceutically acceptable salt thereof, is administered on an intermittent dosing schedule.

61. The method of claim 60, wherein the intermittent dosing schedule comprises 5 days with dosing and 2 days without dosing in each of one or more dosing weeks.

62. A method of treating chemotherapy-resistant ovarian cancer comprising:
determining or having determined whether a subject has a homologous repair deficiency (HRD) or a HRD-positive (HRD+) status; and
if the subject has the HRD or the HRD-positive (HRD+) status, administering an effective dose of azenosertib, or a pharmaceutically acceptable salt thereof, to the subject;
wherein the administration of azenosertib, or a pharmaceutically acceptable salt thereof, results in the inhibition of the cancer in the subject.

63. The method of claim 62, wherein the effective dose of azenosertib, or a pharmaceutically acceptable salt thereof, is at least about 250 mg/day, or an equivalent thereof.

64. The method of claim 62 or 63, wherein the effective dose of azenosertib, or a pharmaceutically acceptable salt thereof, is about 350 to about 400 mg/day, or an equivalent thereof.

65. The method of any one of claims 62 to 64, wherein the effective dose of azenosertib, or a pharmaceutically acceptable salt thereof, is administered on an intermittent dosing schedule.

66. The method of claim 65, wherein the intermittent dosing schedule comprises 5 days with dosing and 2 days without dosing in each of one or more dosing weeks.

67. The method of any one of claims 62 to 66, wherein the HRD or HRD-positive (HRD+ status) is due to the cancer having a mutation in BRCA1 and/or BRCA2.

68. The method of any one of claims 1 to 67, wherein the treatment results in response rate at or greater than 10%, 15%, 20%, 25%, 30%, 35%, 40%, 45% or 50%.

69. The method of claim 68, wherein the response rate is measured by complete response (CR), partial response (PR), CA-125 50% response, or combination thereof.

70. The method of any one of claims 1 to 69, wherein the treatment results in progression-free survival (PFS) of 5 months, 6 months, 7 months, 8 months, 9 months, 10 months, 11 months, 12 months or longer.

71. The method of any one of claims 1 to 70, wherein the method further comprises first determining the HRD status prior to selecting the subject.

72. The method of claims 1 to 71, wherein the HRD comprises a copy number variation, a somatic copy number alteration (SCNA), an aneuploidy, a loss of heterozygosity (LOH), a large-scale transition (LST), a telomeric allelic imbalance (TAI) or a combination thereof.

73. The method of any one of claims 1 to 72, wherein the HRD-positive (HRD+) status is determined using a functional assay or genome sequencing.

74. The method of any one of claims 1 to 72, wherein the HRD-positive (HRD+) status is determined using an HRD score.

75. The method of any one of claims 5 to 74, wherein the inhibition of the cancer is measured by inhibition of tumor growth.

76. The method of any one of claims 5 to 75, wherein the inhibition of the cancer is measured by reduction of tumor volume.
